SEQUENCE LISTING (113 sequences)

SEQ ID NO: 1 - protein, 207 aa, synthetic construct
  REGION 1..207: note = synthetically constructed nanostructure polypeptide
MEGMDPLAVLAESRLLPLLTVRGGEDLAGLATVLELMGVGALEITLRTEKGLEALKALRKSGLLLGAGTVRSPKEAEAALEAGAAFLVSPGLLEEVAALAQARGVPYLPGVLTPTEVERALALGLSALKFFPAEPFQGVRVLRAYAEVFPEVRFLPTGGIKEEHLPHYAALPNLLAVGGSWLLQGDLAAVMKKVKAAKALLSPQAPG

SEQ ID NO: 2 - protein, 156 aa, synthetic construct
  REGION 1..156: note = synthetically constructed nanostructure polypeptide
MTKKVGIVDTTFARVDMAEAAIRTLKALSPNIKIIRKTVPGIKDLPVACKKLLEEEGCDIVMALGMPGKAEKDKVCAHEASLGLMLAQLMTNKHIIEVFVHEDEAKDDDELDILALVRAIEHAANVYYLLFKPEYLTRMAGKGLRQGREDAGPARE

SEQ ID NO: 3 - protein, 156 aa, synthetic construct
  REGION 1..156: note = synthetically constructed nanostructure polypeptide
MTKKVGIVDTTFARVDMASAAILTLKMESPNIKIIRKTVPGIKDLPVACKKLLEEEGCDIVMALGMPGKAEKDKVCAHEASLGLMLAQLMTNKHIIEVFVHEDEAKDDAELKILAARRAIEHALNVYYLLFKPEYLTRMAGKGLRQGFEDAGPARE

SEQ ID NO: 4 - protein, 209 aa, synthetic construct
  REGION 1..209: note = synthetically constructed nanostructure polypeptide
MSTINNQLKALKVIPVIAIDNAEDIIPLGKVLAENGLPAAEITFRSSAAVKAIMLLRSAQPEMLIGAGTILNGVQALAAKEAGATFVVSPGFNPNTVRACQIIGIDIVPGVNNPSTVEAALEMGLTTLKFFPAEASGGISMVKSLVGPYGDIRLMPTGGITPSNIDNYLAIPQVLACGGTWMVDKKLVTNGEWDEIARLTREIVEQVNP

SEQ ID NO: 5 - protein, 114 aa, synthetic construct
  REGION 1..114: note = synthetically constructed nanostructure polypeptide
MPIFTLNTNIKATDVPSDFLSLTSRLVGLILSKPGSYVAVHINTDQQLSFGGSTNPAAFGTLMSIGGIEPSKNRDHSAVLFDHLNAMLGIPKNRMYIHFVNLNGDDVGWNGTTF

SEQ ID NO: 6 - protein, 157 aa, synthetic construct
  REGION 1..157: note = synthetically constructed nanostructure polypeptide
MNQHSHKDYETVRIAVVRARWHADIVDACVEAFEIAMAAIGGDRFAVDVFDVPGAYEIPLHARTLAETGRYGAVLGTAFVVNGGIYRHEFVASAVIDGMMNVQLSTGVPVLSAVLTPHRYRDSAEHHRFFAAHFAVKGVEAARACIEILAAREKIAA

SEQ ID NO: 7 - protein, 205 aa, synthetic construct
  REGION 1..205: note = synthetically constructed nanostructure polypeptide
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 8 - protein, 157 aa, synthetic construct
  REGION 1..157: note = synthetically constructed nanostructure polypeptide
MNQHSHKDYETVRIAVVRARWHAEIVDACVSAFEAAMADIGGDRFAVDVFDVPGAYEIPLHARTLAETGRYGAVLGTAFVVNGGIYRHEFVASAVIDGMMNVQLSTGVPVLSAVLTPHRYRDSDAHTLLFLALFAVKGMEAARACVEILAAREKIAA

SEQ ID NO: 9 - protein, 177 aa, synthetic construct
  REGION 1..177: note = synthetically constructed nanostructure polypeptide
MFTKSGDDGNTNVINKRVGKDSPLVNFLGDLDELNSFIGFAISKIPWEDMKKDLERVQVELFEIGEDLSTQSSKKKIDESYVLWLLAATAIYRIESGPVKLFVIPGGSEEASVLHVTRSVARRVERNAVKYTKELPEINRMIIVYLNRLSSLLFAMALVANKRRNQSEKIYEIGKSW

SEQ ID NO: 10 - protein, 157 aa, synthetic construct
  REGION 1..157: note = synthetically constructed nanostructure polypeptide
MNQHSHKDYETVRIAVVRARWHADIVDQCVRAFEEAMADAGGDRFAVDVFDVPGAYEIPLHARTLAETGRYGAVLGTAFVVNGGIYRHEFVASAVIDGMMNVQLSTGVPVLSAVLTPHRYRSSREHHEFFREHFMVKGVEAAAACITILAAREKIAA

SEQ ID NO: 11 - protein, 201 aa, synthetic construct
  REGION 1..201: note = synthetically constructed nanostructure polypeptide
MGHTKGPTPQQHDGSALRIGIVHARWNKTIIMPLLIGTIAKLLECGVKASNIVVQSVPGSWELPIAVQRLYSASQLQTPSSGPSLSAGDLLGSSTTDLTALPTTTASSTGPFDALIAIGVLIKGETMHFEYIADSVSHGLMRVQLDTGVPVIFGVLTVLTDDQAKARAGVIEGSHNHGEDWGLAAVEMGVRRRDWAAGKTE

SEQ ID NO: 12 - protein, 237 aa, synthetic construct
  REGION 1..237: note = synthetically constructed nanostructure polypeptide
MYEVDHADVYDLFYLGRGKDYAAEASDIADLVRSRTPEASSLLDVACGTGTHLEHFTKEFGDTAGLELSEDMLTHARKRLPDATLHQGDMRDFQLGRKFSAVVSMFSSVGYLKTVAELGAAVASFAEHLEPGGVVVVEPWWFPETFADGWVSADVVRRDGRTVARVSHSVREGNATRMEVHFTVADPGKGVRHFSDVHLITLFHQREYEAAFMAAGLRVEYLEGGPSGRGLFVGVPA

SEQ ID NO: 13 - protein, 138 aa, synthetic construct
  REGION 1..138: note = synthetically constructed nanostructure polypeptide
MGMKEKFVLIITHGDFGKGLLSGAEVIIGKQENVHTVGLNLGDNIEKVAKEVMRIIIAKLAEDKEIIIVVDLFGGSPFNIALEMMKTFDVKVITGINMPMLVELLTSINVYDTTELLENISKIGKDGIKVIEKSSLKM

SEQ ID NO: 14 - protein, 154 aa, synthetic construct
  REGION 1..154: note = synthetically constructed nanostructure polypeptide
MKYDGSKLRIGILHARWNLEIIAALVAGAIKRLQEFGVKAENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 15 - protein, 164 aa, synthetic construct
  REGION 1..164: note = synthetically constructed nanostructure polypeptide
MAVKGLGEVDQKYDGSKLRIGILHARWNRKIILALVAGAVLRLLEFGVKAENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 16 - protein, 175 aa, synthetic construct
  REGION 1..175: note = synthetically constructed nanostructure polypeptide
MGANWYLDNESSRLSFTSTKNADIAEVHRFLVLHGKVDPKGLAEVEVETESISTGIPLRDMLLRVLVFQVSKFPVAQINAQLDMRPINNLAPGAQLELRLPLTVSLRGKSHSYNAELLATRLDERRFQVVTLEPLVIHAQDFDMVRAFNALRLVAGLSAVSLSVPVGAVLIFTAR

SEQ ID NO: 17 - protein, 208 aa, synthetic construct
  REGION 1..208: note = synthetically constructed nanostructure polypeptide
MTDYIRDGSAIKALSFAIILAEADLRHIPQDLQRLAVRVIHACGMVDVANDLAFSEGAGKAGRNALLAGAPILCDARMVAEGITRSRLPADNRVIYTLSDPSVPELAKKIGNTRSAAALDLWLPHIEGSIVAIGNAPTALFRLFELLDAGAPKPALIIGMPVGFVGAAESKDELAANSRGVPYVIVRGRRGGSAMTAAAVNALASERE

SEQ ID NO: 18 - protein, 128 aa, synthetic construct
  REGION 1..128: note = synthetically constructed nanostructure polypeptide
MITVFGLKSKLAPRREKLAEVIYSSLHLGLDIPKGKHAIRFLCLEKEDFYYPFDRSDDYTVIEINLMAGRSEETKMLLIFLLFIALERKLGIRAHDVEITIKEQPAHCWGFRGRTGDSARDLDYDIYV

SEQ ID NO: 19 - protein, 235 aa, synthetic construct
  REGION 1..235: note = synthetically constructed nanostructure polypeptide
MGSDLQKLQRFSTCDISDGLLNVYNIPTGGYFPNLTAISPPQNSSIVGTAYTVLFAPIDDPRPAVNYIDSVPPNSILVLALEPHLQSQFHPFIKITQAMYGGLMSTRAQYLKSNGTVVFGRIRDVDEHRTLNHPVFAYGVGSCAPKAVVKAVGTNVQLKILTSDGVTQTICPGDYIAGDNNGIVRIPVQETDISKLVTYIEKSIEVDRLVSEAIKNGLPAKAAQTARRMVLKDYI

SEQ ID NO: 20 - protein, 162 aa, synthetic construct
  REGION 1..162: note = synthetically constructed nanostructure polypeptide
MSGMRVYLGADHAGYELKQAIIAFLKMTGHEPIDCGALRYDADDDYPAFCIAAATRTVADPGSLGIVLGGSGNGEQIAANKVPGARCALAWSVQTAALAREHNNAQLIGIGGRMHTLEEALRIVKAFVTTPWSKAQRHQRRIDILAEYERTHEAPPVPGAPA

SEQ ID NO: 21 - protein, 157 aa, synthetic construct
  REGION 1..157: note = synthetically constructed nanostructure polypeptide
MGDDARIAAIGDVDELNSQIGVLLAEPLPDDVRAALSAIQHDLFDLGGELCIPGHAAITEDHLLRLALWLVHYNGQLPPLEEFILPGGARGAALAHVCRTVCRRAERSIKALGASEPLNIAPAAYVNLLSDLLFVLARVLNRAAGGADVLWDRTRAH

SEQ ID NO: 22 - protein, 157 aa, synthetic construct
  REGION 1..157: note = synthetically constructed nanostructure polypeptide
MILSAEQSFTLRHPHGQAAALAFVREPAAALAGVQRLRGLDSDGEQVWGELLVRVPLLGEVDLPFRSEIVRTPQGAELRPLTLTGERAWVAVSGQATAAEGGEMAFAFQFQAHLATPEAEGEGGAAFEVMVQAAAGVTLLLVAMALPQGLAAGLPPA

SEQ ID NO: 23 - protein, 156 aa, synthetic construct
  REGION 1..156: note = synthetically constructed nanostructure polypeptide
MTKKVGIVDTTFARVDMASAAILTLKMESPNIKIIRKTVPGIKDLPVACKKLLEEEGCDIVMALGMPGKKEKDKVCAHEASLGLMLAQLMTNKHIIEVFVHEDEAKDDAELKILAARRAIEHALNVYYLLFKPEYLTRMAGKGLRQGFEDAGPARE

SEQ ID NO: 24 - protein, 209 aa, synthetic construct
  REGION 1..209: note = synthetically constructed nanostructure polypeptide
MDDINNQLKRLKVIPVIAIDNAEDIIPLGKVLAENGLPAAEITFRSSAAVKAIMLLRSAQPEMLIGAGTILNGVQALAAKEAGADFVVSPGFNPNTVRACQIIGIDIVPGVNNPSTVEQALEMGLTTLKFFPAEASGGISMVKSLVGPYGDIRLMPTGGITPDNIDNYLAIPQVLACGGTWMVDKKLVRNGEWDEIARLTREIVEQVNP

SEQ ID NO: 25 - protein, 114 aa, synthetic construct
  REGION 1..114: note = synthetically constructed nanostructure polypeptide
MPIFTLNTNIKADDVPSDFLSLTSRLVGLILSKPGSYVAVHINTDQQLSFGGSTNPAAFGTLMSIGGIEPDKNRDHSAVLFDHLNAMLGIPKNRMYIHFVNLNGDDVGWNGTTF

SEQ ID NO: 26 - protein, 114 aa, synthetic construct
  REGION 1..114: note = synthetically constructed nanostructure polypeptide
MPIFTLNTNIKADDVPSDFLSLTSRLVGLILSEPGSYVAVHINTDQQLSFGGSTNPAAFGTLMSIGGIEPDKNEDHSAVLFDHLNAMLGIPKNRMYIHFVDLDGDDVGWNGTTF

SEQ ID NO: 27 - protein, 157 aa, synthetic construct
  REGION 1..157: note = synthetically constructed nanostructure polypeptide
MNQHSHKDHETVRIAVVRARWHADIVDACVEAFEIAMAAIGGDRFAVDVFDVPGAYEIPLHARTLAETGRYGAVLGTAFVVNGGIYRHEFVASAVIDGMMNVQLDTGVPVLSAVLTPHRYRDSDEHHRFFAAHFAVKGVEAARACIEILNAREKIAA

SEQ ID NO: 28 - protein, 157 aa, synthetic construct
  REGION 1..157: note = synthetically constructed nanostructure polypeptide
MNQHSHKDHETVRIAVVRARWHADIVDACVEAFEIAMAAIGGDRFAVDVFDVPGAYEIPLHARTLAETGRYGAVLGTAFVVDGGIYDHEFVASAVIDGMMNVQLDTGVPVLSAVLTPHEYEDSDEDHEFFAAHFAVKGVEAARACIEILNAREKIAA

SEQ ID NO: 29 - protein, 205 aa, synthetic construct
  REGION 1..205: note = synthetically constructed nanostructure polypeptide
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPQFVKAMKGPFPNVKFVPTGGVNLDNVCEWFKAGVLAVGVGDALVKGDPDEVREKAKKFVEKIRGCTE

SEQ ID NO: 30 - protein, 205 aa, synthetic construct
  REGION 1..205: note = synthetically constructed nanostructure polypeptide
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVEAMKGPFPNVKFVPTGGVDLDDVCEWFDAGVLAVGVGDALVEGDPDEVREDAKEFVEEIRGCTE

SEQ ID NO: 31 - protein, 205 aa, synthetic construct
  REGION 1..205: note = synthetically constructed nanostructure polypeptide
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGKALVKGKPDEVREKAKKFVKKIRGCTE

SEQ ID NO: 32 - protein, 157 aa, synthetic construct
  REGION 1..157: note = synthetically constructed nanostructure polypeptide
MNQHSHKDHETVRIAVVRARWHAEIVDACVSAFEAAMRDIGGDRFAVDVFDVPGAYEIPLHARTLAETGRYGAVLGTAFVVNGGIYRHEFVASAVIDGMMNVQLDTGVPVLSAVLTPHRYRDSDAHTLLFLALFAVKGMEAARACVEILAAREKIAA

SEQ ID NO: 33 - protein, 157 aa, synthetic construct
  REGION 1..157: note = synthetically constructed nanostructure polypeptide
MNQHSHKDHETVRIAVVRARWHAEIVDACVSAFEAAMRDIGGDRFAVDVFDVPGAYEIPLHARTLAETGRYGAVLGTAFVVDGGIYDHEFVASAVIDGMMNVQLDTGVPVLSAVLTPHEYEDSDADTLLFLALFAVKGMEAARACVEILAAREKIAA

SEQ ID NO: 34 - protein, 157 aa, synthetic construct
  REGION 1..157: note = synthetically constructed nanostructure polypeptide
MNQHSHKDHETVRIAVVRARWHAEIVDACVSAFEAAMRDIGGDRFAVDVFDVPGAYEIPLHARTLAETGRYGAVLGTAFVVNGGIYRHEFVASAVINGMMNVQLNTGVPVLSAVLTPHNYDKSKAHTLLFLALFAVKGMEAARACVEILAAREKIAA

SEQ ID NO: 35 - protein, 156 aa, synthetic construct
  REGION 1..156: note = synthetically constructed nanostructure polypeptide
  SITE 70: note = misc_feature - Xaa is Ala or Lys
MTKKVGIVDTTFARVDMASAAILTLKMESPNIKIIRKTVPGIKDLPVACKKLLEEEGCDIVMALGMPGKXEKDKVCAHEASLGLMLAQLMTNKHIIEVFVHEDEAKDDAELKILAARRAIEHALNVYYLLFKPEYLTRMAGKGLRQGFEDAGPARE

SEQ ID NO: 36 - protein, 209 aa, synthetic construct
  REGION 1..209: note = synthetically constructed nanostructure polypeptide
  SITE 2: note = misc_feature - Xaa is Ser or Asp
  SITE 3: note = misc_feature - Xaa is Thr or Asp
  SITE 10: note = misc_feature - Xaa is Ala or Arg
  SITE 85: note = misc_feature - Xaa is Thr or Asp
  SITE 119: note = misc_feature - Xaa is Ala or Gln
  SITE 163: note = misc_feature - Xaa is Ser or Asp
  SITE 189: note = misc_feature - Xaa is Thr or Arg
MXXINNQLKXLKVIPVIAIDNAEDIIPLGKVLAENGLPAAEITFRSSAAVKAIMLLRSAQPEMLIGAGTILNGVQALAAKEAGAXFVVSPGFNPNTVRACQIIGIDIVPGVNNPSTVEXALEMGLTTLKFFPAEASGGISMVKSLVGPYGDIRLMPTGGITPXNIDNYLAIPQVLACGGTWMVDKKLVXNGEWDEIARLTREIVEQVNP

SEQ ID NO: 37 - protein, 114 aa, synthetic construct
  REGION 1..114: note = synthetically constructed nanostructure polypeptide
  SITE 13: note = misc_feature - Xaa is Thr or Asp
  SITE 33: note = misc_feature - Xaa is Lys or Glu
  SITE 71: note = misc_feature - Xaa is Ser or Asp
  SITE 74: note = misc_feature - Xaa is Arg or Glu
  SITE 101: note = misc_feature - Xaa is Asn or Asp
  SITE 103: note = misc_feature - Xaa is Asn or Asp
MPIFTLNTNIKAXDVPSDFLSLTSRLVGLILSXPGSYVAVHINTDQQLSFGGSTNPAAFGTLMSIGGIEPXKNXDHSAVLFDHLNAMLGIPKNRMYIHFVXLXGDDVGWNGTTF

SEQ ID NO: 38 - protein, 157 aa, synthetic construct
  REGION 1..157: note = synthetically constructed nanostructure polypeptide
  SITE 9: note = misc_feature - Xaa is Tyr or His
  SITE 82: note = misc_feature - Xaa is Asn or Asp
  SITE 87: note = misc_feature - Xaa is Arg or Asp
  SITE 105: note = misc_feature - Xaa is Ser or Asp
  SITE 119: note = misc_feature - Xaa is Arg or Glu
  SITE 121: note = misc_feature - Xaa is Arg or Glu
  SITE 124: note = misc_feature - Xaa is Ala or Asp
  SITE 126: note = misc_feature - Xaa is His or Asp
  SITE 128: note = misc_feature - Xaa is Arg or Glu
  SITE 150: note = misc_feature - Xaa is Ala or Asn
MNQHSHKDXETVRIAVVRARWHADIVDACVEAFEIAMAAIGGDRFAVDVFDVPGAYEIPLHARTLAETGRYGAVLGTAFVVXGGIYXHEFVASAVIDGMMNVQLXTGVPVLSAVLTPHXYXDSXEXHXFFAAHFAVKGVEAARACIEILXAREKIAA

SEQ ID NO: 39 - protein, 205 aa, synthetic construct
  REGION 1..205: note = synthetically constructed nanostructure polypeptide
  SITE 126: note = misc_feature - Xaa is Thr or Asp
  SITE 139: note = misc_feature - Xaa is Gln or Glu
  SITE 142: note = misc_feature - Xaa is Lys or Glu
  SITE 160: note = misc_feature - Xaa is Asn or Asp
  SITE 163: note = misc_feature - Xaa is Asn or Asp
  SITE 166: note = misc_feature - Xaa is Glu or Lys
  SITE 169: note = misc_feature - Xaa is Lys or Asp
  SITE 179: note = misc_feature - Xaa is Ser, Lys or Asp
  SITE 183: note = misc_feature - Xaa is Lys or Glu
  SITE 185: note = misc_feature - Xaa is Thr, Asp or Lys
  SITE 192: note = misc_feature - Xaa is Lys or Asp
  SITE 195: note = misc_feature - Xaa is Ala, Glu or Lys
  SITE 198: note = misc_feature - Xaa is Glu or Lys
  SITE 199: note = misc_feature - Xaa is Lys or Glu
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHXILKLFPGEVVGPXFVXAMKGPFPNVKFVPTGGVXLDXVCXWFXAGVLAVGVGXALVXGXPDEVREXAKXFVXXIRGCTE

SEQ ID NO: 40 - protein, 157 aa, synthetic construct
  REGION 1..157: note = synthetically constructed nanostructure polypeptide
  SITE 9: note = misc_feature - Xaa is Tyr or His
  SITE 38: note = misc_feature - Xaa is Ala or Arg
  SITE 82: note = misc_feature - Xaa is Asn or Asp
  SITE 87: note = misc_feature - Xaa is Arg or Asp
  SITE 97: note = misc_feature - Xaa is Asp or Asn
  SITE 105: note = misc_feature - Xaa is Ser, Asp or Asn
  SITE 119: note = misc_feature - Xaa is Arg, Glu or Asn
  SITE 121: note = misc_feature - Xaa is Arg, Asp or Glu
  SITE 122: note = misc_feature - Xaa is Asp or Lys
  SITE 124: note = misc_feature - Xaa is Asp or Lys
  SITE 126: note = misc_feature - Xaa is His or Asp
MNQHSHKDXETVRIAVVRARWHAEIVDACVSAFEAAMXDIGGDRFAVDVFDVPGAYEIPLHARTLAETGRYGAVLGTAFVVXGGIYXHEFVASAVIXGMMNVQLXTGVPVLSAVLTPHXYXXSXAXTLLFLALFAVKGMEAARACVEILAAREKIAA

SEQ ID NO: 41 - protein, 159 aa, synthetic construct
  REGION 1..159: note = synthetically constructed nanostructure polypeptide
MGEVPIGDPKELNGMEIAAVYLQPIEMEPRGIDLAASLADIHLEADIHALKNNPNGFPEGFWMPYLTIAYALANADTGAIKTGTLMPMVADDGPHYGANIAMEKDKKGGFGVGTYALTFLISNPEKQGFGRHVDEETGVGKWFEPFVVTYFFKYTGTPK

SEQ ID NO: 42 - protein, 184 aa, synthetic construct
  REGION 1..184: note = synthetically constructed nanostructure polypeptide
MSQAIGILELTSIAKGMELGDAMLKSANVDLLVSKTISPGKFLLMLGGDIGAIQQAIETGTSQAGEMLVDSLVLANIHPSVLPAISGLNSVDKRQAVGIVETWSVAACISAADLAVKGSNVTLVRVHMAFGIGGKCYMVVAGDVLDVAAAVATASLAAGAKGLLVYASIIPRPHEAMWRQMVEG

SEQ ID NO: 43 - protein, 103 aa, synthetic construct
  REGION 1..103: note = synthetically constructed nanostructure polypeptide
MEEVVLITVPSALVAVKIAHALVEERLAACVNIVPGLTSIYRWQGSVVSDHELLLLVKTTTHAFPKLKERVKALHPYTVPEIVALPIAEGNREYLDWLRENTG

SEQ ID NO: 44 - protein, 122 aa, synthetic construct
  REGION 1..122: note = synthetically constructed nanostructure polypeptide
MVRGIRGAITVEEDTPAAILAATIELLLKMLEANGIQSYEELAAVIFTVTEDLTSAFPAEAARLIGMHRVPLLSAREVPVPGSLPRVIRVLALWNTDTPQDRVRHVYLNEAVRLRPDLESAQ

SEQ ID NO: 45 - protein, 177 aa, synthetic construct
  REGION 1..177: note = synthetically constructed nanostructure polypeptide
MSKAKIGIVTVSDRASAGITADISGKAIILALNLYLTSEWEPIYQVIPDEQDVIETTLIKMADEQDCCLIVTTGGTGPAKRDVTPEATEAVCDRMMPGFGELMRAESLKEVPTAILSRQTAGLRGDSLIVNLPGDPASISDCLLAVFPAIPYCIDLMEGPYLECNEAMIKPFRPKAK

SEQ ID NO: 46 - protein, 122 aa, synthetic construct
  REGION 1..122: note = synthetically constructed nanostructure polypeptide
MVRGIRGAITVNSDTPTSIIIATILLLEKMLEANGIQSYEELAAVIFTVTEDLTSAFPAEAARQIGMHRVPLLSAREVPVPGSLPRVIRVLALWNTDTPQDRVRHVYLSEAVRLRPDLESAQ

SEQ ID NO: 47 - protein, 172 aa, synthetic construct
  REGION 1..172: note = synthetically constructed nanostructure polypeptide
MRITTKVGDKGSTRLFGGEEVWKDSPIIEANGTLDELTSFIGEAKHYVDEEMKGILEEIQNDIYKIMGEIGSKGKIEGISEERIAWLLKLILRYMEMVNLKSFVLPGGTLESAKLDVCRTIARRALRKVLTVTREFGIGAEAAAYLLALSDLLFLLARVIEIEKNKLKEVRS

SEQ ID NO: 48 - protein, 123 aa, synthetic construct
  REGION 1..123: note = synthetically constructed nanostructure polypeptide
MPHLVIEATANLRLETSPGELLEQANKALFASGQFGEADIKSRFVTLEAYRQGTAAVERAYLHACLSILDGRDIATRTLLGASLCAVLAEAVAGGGEEGVQVSVEVREMERLSYAKRVVARQR

SEQ ID NO: 49 - protein, 158 aa, synthetic construct
  REGION 1..158: note = synthetically constructed nanostructure polypeptide
MESVNTSFLSPSLVTIRDFDNGQFAVLRIGRTGFPADKGDIDLCLDKMIGVRAAQIFLGDDTEDGFKGPHIRIRCVDIDDKHTYNAMVYVDLIVGTGASEVERETAEEEAKLALRVALQVDIADEHSCVTQFEMKLREELLSSDSFHPDKDEYYKDFL

SEQ ID NO: 50 - protein, 113 aa, synthetic construct
  REGION 1..113: note = synthetically constructed nanostructure polypeptide
MPVIQTFVSTPLDHHKRLLLAIIYRIVTRVVLGKPEDLVMMTFHDSTPMHFFGSTDPVACVRVEALGGYGPSEPEKVTSIVTAAITAVCGIVADRIFVLYFSPLHCGWNGTNF

SEQ ID NO: 51 - protein, 103 aa, synthetic construct
  REGION 1..103: note = synthetically constructed nanostructure polypeptide
MEEVVLITVPSALVAVKIAHALVEERLAACVNIVPGLTSIYREEGSVVSDHELLLLVKTTTDAFPKLKERVKELHPYEVPEIVALPIAEGNREYLDWLRENTG

SEQ ID NO: 52 - protein, 869 aa, Human immunodeficiency virus 1
MRVKGIKKNYQHWWRGGIMLLGMLMICSSAEKLWVTVYYGVPVWKEATTTLFCASDAKAQNPEMHNIWATHACVPTDPNPQEVILKNLTEEFNMWKNNMVEQMHEDIISLWDQSLKPCVKLTPLCVTLNCTNAESLNCTATNGTNNCSASTKPMEEMKNCSFNITTSVQDKKQQEYALFYKLDIIPIDNNENDLNNTNYTSYRLISCNTSVITQACPKITFEPIPIHYCAPAGFAILKCKDKRFNGTGPCKNVSTVQCTHGIRPVVSTQLLLNGSLAEEGVVLRSENFTDNAKNIIVQLKDPVNITCTRPNNNTRKSITIGPGRAFYATGQVIGDIRKAHCDLNGTEWDNALKQIVEELRKQYGNNITIFNSSSGGDPEIVMHSFNCGGEFFYCNTAQLFNSTWLFNSTWNSTERLGNDTERTNDTITLPCKIKQVINMWQTVGKAMYAPPIRGLIRCSSNITGLILTRDGSGNTTGNETFRPGGGNMKDNWRSELYKYKVVKIEPLGVAPTRAKRRVVQREKRAAGLGALFLGFLGMAGSTMGAASLTLTVQARQLLSGIVQQQNNLLRAIEAQQHLLQLTVWGIKQLQARVLAVERYLRDQQLLGIWGCSGKLICTTTVPWNASWSNKSLDNIWENMTWMQWEKEIDNYTDVIYKLLEESQNQQEKNEQELLELDKWASLWNWFDITRWLWYIKIFIMIVGGLVGLRIVFAVLSIVNRVRQGYSPLSFQTLFPAPRGPDRPEGTEEGGGERGRDSSDRSAHGFLALIWGDLWSLCLFSYRRLRDLLLIAARIVELLGRRGWEVLKYWWSLLQYWSQELKKSAVSLLNATAIAVAEGTDRIIEIVQRAGRAIIHIPRRIRQGAERALL

SEQ ID NO: 53 - protein, 492 aa, Human immunodeficiency virus 1
LWVTVYYGVPVWKEATTTLFCASDAKAQNPEMHNIWATHACVPTDPNPQEVILKNLTEEFNMWKNNMVEQMHEDIISLWDQSLKPCVKLTPLCVTLNCTNAESLNCTATNGTNNCSASTKPMEEMKNCSFNITTSVQDKKQQEYALFYKLDIIPIDNNENDLNNTNYTSYRLISCNTSVITQACPKITFEPIPIHYCAPAGFAILKCKDKRFNGTGPCKNVSTVQCTHGIRPVVSTQLLLNGSLAEEGVVLRSENFTDNAKNIIVQLKDPVNITCTRPNNNTRKSITIGPGRAFYATGQVIGDIRKAHCDLNGTEWDNALKQIVEELRKQYGNNITIFNSSSGGDPEIVMHSFNCGGEFFYCNTAQLFNSTWLFNSTWNSTERLGNDTERTNDTITLPCKIKQVINMWQTVGKAMYAPPIRGLIRCSSNITGLILTRDGSGNTTGNETFRPGGGNMKDNWRSELYKYKVVKIEPLGVAPTRAKRRVVQREKR

SEQ ID NO: 54 - protein, 191 aa, Human immunodeficiency virus 1
MGAASLTLTVQARQLLSGIVQQQNNLLRAIEAQQHLLQLTVWGIKQLQARVLAVERYLRDQQLLGIWGCSGKLICTTTVPWNASWSNKSLDNIWENMTWMQWEKEIDNYTDVIYKLLEESQNQQEKNEQELLELDKWASLWNWFDITRWLWYIKIFIMIVGGLVGLRIVFAVLSIVNRVRQGYSPLSFQTL

SEQ ID NO: 55 - protein, 22 aa, Human immunodeficiency virus 1
ELDKWASLWNWFDITRWLWYIK

SEQ ID NO: 56 - protein, 574 aa, Respiratory syncytial virus type A
MELPILKTNAITTILAAVTLCFASSQNITEEFYQSTCSAVSKGYLSALRTGWYTSVITIELSNIKENKCNGTDAKVKLIKQELDKYKNAVTELQLLMQSTPAANNRARRELPRFMNYTLNNTKNNNVTLSKKRKRRFLGFLLGVGSAIASGIAVSKVLHLEGEVNKIKNALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNVGKSTTNIMITTIIIVIIVILLLLIAVGLFLYCKARSTPVTLSKDQLSGINNIAFSN

SEQ ID NO: 57 - protein, 566 aa, Influenza A virus
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 58 - protein, 584 aa, Influenza B virus
MKAIIVLLMVVTSNADRICTGITSSNSPHVVKTATQGEVNVTGVIPLTTTPTKSYFANLKGTKTRGKLCPDCLNCTDLDVALGRPMCVGTTPSAKASILHEVRPVTSGCFPIMHDRTKIRQLANLLRGYENIRLSTQNVIDAEKAPGGPYRLGTSGSCPNATSKSGFFATMAWAVPKDNNKNATNPLTVEVPYICAEGEDQITVWGFHSDNKTQMKNLYGDSNPQKFTSSANGVTTHYVSQIGGFPDQTEDGGLPQSGRIVVDYMMQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 59 - protein, 907 aa, Epstein-Barr virus
MEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATSPTLGKTSPTSAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLSMLVLQWASLAVLTLLLLLVMADCAFRRNLSTSHTYTTPPYDDAETYV

SEQ ID NO: 60 - protein, 906 aa, Human cytomegalovirus
MESRIWCLVVCVNLCIVCLGAAVSSSSTSHATSSTHNGSHTSRTTSAQTRSVYSQHVTSSEAVSHRANETIYNTTLKYGDVVGVNTTKYPYRVCSMAQGTDLIRFERNIICTSMKPINEDLDEGIMVVYKRNIVAHTFKVRVYQKVLTFRRSYAYIYTTYLLGSNTEYVAPPMWEIHHINKFAQCYSSYSRVIGGTVFVAYHRDSYENKTMQLIPDDYSNTHSTRYVTVKDQWHSRGSTWLYRETCNLNCMLTITTARSKYPYHFFATSTGDVVYISPFYNGTNRNASYFGENADKFFIFPNYTIVSDFGRPNAAPETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETSGGLVVFWQGIKQKSLVELERLANRSSLNITHRTRRSTSDNNTTHLSSMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKNPFGAFTIILVAIAVVIITYLIYTRQRRLCTQPLQNLFPYLVSADGTTVTSGSTKDTSLQAPPSYEESVYNSGRKGPGPPSSDASTAAPPYTNEQAYQMLLALARLDAEQRAQQNGTDSLDGQTGTQDKGQKPNLLDRLRHRKNGYRHLKDSDEEENV

SEQ ID NO: 61 - protein, 171 aa, Human cytomegalovirus
MSPKDLTPFLTTLWLLLGHSRVPRVRAEECCEFINVNHPPERCYDFKMCNRFTVALRCPDGEVCYSPEKTAEIRGIVTTMTHSLTRQVVHNKLTSCNYNPLYLEADGRIRCGKVNDKAQYLLGAAGSVPYRWINLEYDKITRIVGLDQYLESVKKHKRLDVCRAKMGYMLQ

SEQ ID NO: 62 - protein, 214 aa, Human cytomegalovirus
MLRLLLRHHFHCLLLCAVWATPCLASPWSTLTANQNPSPPWSKLTYSKPHDAATFYCPFLYPSPPRSPLQFSGFQRVSTGPECRNETLYLLYNREGQTLVERSSTWVKKVIWYLSGRNQTILQRMPRTASKPSDGNVQISVEDAKIFGAHMVPKQTKLLRFVVNDGTRYQMCVMKLESWAHVFRDYSVSFQVRLTFTEANNQTYTFCTHPNLIV

SEQ ID NO: 63 - protein, 129 aa, Human cytomegalovirus
MRLCRVWLSVCLCAVVLGQCQRETAEKNDYYRVPHYWDACSRALPDQTRYKYVEQLVDLTLNYHYDASHGLDNFDVLKRINVTEVSLLISDFRRQNRRGGTNKRTTFNAAGSLAPHARSLEFSVRLFAN

SEQ ID NO: 64 - protein, 743 aa, Human cytomegalovirus
MRPGLPPYLTVFTVYLLSHLPSQRYGADAASEALDPHAFHLLLNTYGRPIRFLRENTTQCTYNSSLRNSTVVRENAISFNFFQSYNQYYVFHMPRCLFAGPLAEQFLNQVDLTETLERYQQRLNTYALVSKDLASYRSFSQQLKAQDSLGQQPTTVPPPIDLSIPHVWMPPQTTPHDWKGSHTTSGLHRPHFNQTCILFDGHDLLFSTVTPCLHQGFYLMDELRYVKITLTEDFFVVTVSIDDDTPMLLIFGHLPRVLFKAPYQRDNFILRQTEKHELLVLVKKAQLNRHSYLKDSDFLDAALDFNYLDLSALLRNSFHRYAVDVLKSGRCQMLDRRTVEMAFAYALALFAAARQEEAGTEISIPRALDRQAALLQIQEFMITCLSQTPPRTTLLLYPTAVDLAKRALWTPDQITDITSLVRLVYILSKQNQQHLIPQWALRQIADFALQLHKTHLASFLSAFARQELYLMGSLVHSMLVHTTERREIFIVETGLCSLAELSHFTQLLAHPHHEYLSDLYTPCSSSGRRDHSLERLTRLFPDATVPATVPAALSILSTMQPSTLETFPDLFCLPLGESFSALTVSEHVSYVVTNQYLIKGISYPVSTTVVGQSLIITQTDSQTKCELTRNMHTTHSITAALNISLENCAFCQSALLEYDDTQGVINIMYMHDSDDVLFALDPYNEVVVSSPRTHYLMLLKNGTVLEVTDVVVDATDSRLLMMSVYALSAIIGIYLLYRMLKTC

SEQ ID NO: 65 - protein, 278 aa, Human cytomegalovirus
MCRRPDCGFSFSPGPVVLLWCCLLLPIVSSVAVSVAPTAAEKVPAECPELTRRCLLGEVFQGDKYESWLRPLVNVTRRDGPLSQLIRYRPVTPEAANSVLLDDAFLDTLALLYNNPDQLRALLTLLSSDTAPRWMTVMRGYSECGDGSPAVYTCVDDLCRGYDLTRLSYGRSIFTEHVLGFELVPPSLFNVVVAIRNEATRTNRAVRLPVSTAAAPEGITLFYGLYNAVKEFCLRHQLDPPLLRHLDKYYAGLPPELKQTRVNLPAHSRYGPQAVDAR

SEQ ID NO: 66 - protein, 272 aa, Borreliella burgdorferi
MKKYLLGIGLILALIACKQNVSSLDEKNSVSVDVPGGMKVLVSKEKNKDGKYDLMATVDNVDLKGTSDKNNGSGILEGVKADKSKVKLTVADDLSKTTLEVLKEDGTVVSRKVTSKDKSTTEAKFNEKGELSEKTMTRANGTTLEYSQMTNEDNAAKAVETLKNGIKFEGNLASGKTAVEIKEGTVTLKREIDKNGKVTVSLNDTASGSKKTASWQESTSTLTISANSKKTKDLVFLTNGTITVQNYDSAGTKLEGSAAEIKKLDELKNALR

SEQ ID NO: 67 - protein, 269 aa, Bordetella pertussis
MRCTRAIRQTARTGWLTWLAILAVTAPVTSPAWADDPPATVYRYDSRPPEDVFQNGFTAWGNNDNVLDHLTGRSCQVGSSNSAFVSTSSSRRYTEVYLEHRMQEAVEAERAGRGTGHFIGYIYEVRADNNFYGAASSYFEYVDTYGDNAGRILAGALATYQSEYLAHRRIPPENIRRVTRVYHNGITGETTTTEYSNARYVSQQTRANPNPYTSRRSVASIVGTLVRMAPVIGACMARQAESSEAMAAWSERAGEAMVLVYYESIAYSF

SEQ ID NO: 68 - protein, 495 aa, Dengue virus
MRCVGIGNRDFVEGLSGATWVDVVLEHGSCVTTMAKDKPTLDIELLKTEVTNPAVLRKLCIEAKISNTTTDSRCPTQGEATLVEEQDTNFVCRRTFVDRGWGNGCGLFGKGSLITCAKFKCVTKLEGKIVQYENLKYSVIVTVHTGDQHQVGNETTEHGTTATITPQAPTSEIQLTDYGALTLDCSPRTGLDFNEMVLLTMEKKSWLVHKQWFLDLPLPWTSGASTSQETWNRQDLLVTFKTAHAKKQEVVVLGSQEGAMHTALTGATEIQTSGTTTIFAGHLKCRLKMDKLTLKGMSYVMCTGSFKLEKEVAETQHGTVLVQVKYEGTDAPCKIPFSSQDEKGVTQNGRLITANPIVTDKEKPVNIEAEPPFGESYIVVGAGEKALKLSWFKKGSSIGKMFEATARGARRMAILGDTAWDFGSIGGVFTSVGKLIHQIFGTAYGVLFSGVSWTMKIGIGILLTWLGLNSRSTSLSMTCIAVGMVTLYLGVMVQA

SEQ ID NO: 69 - protein, 1255 aa, Human SARS coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 70 - protein, 1353 aa, Middle East respiratory syndrome-related coronavirus
MIHSVFLLMFLLTPTESYVDVGPDSIKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFHKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFILCCTGCGTNCMGKLKCNRCCDRYEEYDLEPHKVHVH

SEQ ID NO: 71 - protein, 676 aa, Zaire ebolavirus
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 72 - protein, 681 aa, Lake Victoria marburgvirus
MKTTCFLISLILIQGTKNLPILEIASNNQPQNVDSVCSGTLQKTEDVHLMGFTLSGQKVADSPLEASKRWAFRTGVPPKNVEYTEGEEAKTCYNISVTDPSGKSLLLDPPTNIRDYPKCKTIHHIQGQNPHAQGIALHLWGAFFLYDRIASTTMYRGKVFTEGNIAAMIVNKTVHKMIFSRQGQGYRHMNLTSTNKYWTSSNGTQTNDTGCFGALQEYNSTKNQTCAPSKIPPPLPTARPEIKLTSTPTDATKLNTTDPSSDDEDLATSGSGSGEREPHTTSDAVTKQGLSSTMPPTPSPQPSTPQQGGNNTNHSQDAVTELDKNNTTAQPSMPPHNTTTISTNNTSKHNFSTLSAPLQNTTNDNTQSTITENEQTSAPSITTLPPTGNPTTAKSTSSKKGPATTAPNTTNEHFTSPPPTPSSTAQHLVYFRRKRSILWREGDMFPFLDGLINAPIDFDPVPNTKTIFDESSSSGASAEEDQHASPNISLTLSYFPNINENTAYSGENENDCDAELRIWSVQEDDLAAGLSWIPFFGPGIEGLYTAVLIKNQNNLVCRLRRLANQTAKSLELLLRVTTEERTFSLINRHAIDFLLTRWGGTCKVLGPDCCIGIEDLSKNISEQIDQIKKDEQKEGTGWGLGGKWWTSDWGVLTNLGILLLLSIAVLIALSCICRIFTKYIG

SEQ ID NO: 73 - protein, 630 aa, Hanta virus
LRNVYDMKIECPHTVSFGENSVIGYVELPPVPLADTAQMVPESSCNMDNHQSLNTITKYTQVSWRGKADQSQSSQNSFETVSTEVDLKGTCVLKHKMVEESYRSRKSVTCYDLSCNSTYCKPTLYMIVPIHACNMMKSCLIALGPYRVQVVYERSYCMTGVLIEGKCFVPDQSVVSIIKHGIFDIASVHIVCFFVAVKGNTYKIFEQVKKSFESTCNDTENKVQGYYICIVGGNSAPIYVPTLDDFRSMEAFTGIFRSPHGEDHDLAGEEIASYSIVGPANAKVPHSASSDTLSLIAYSGIPSYSSLSILTSSTEAKHVFSPGLFPKLNHTNCDKSAIPLIWTGMIDLPGYYEAVHPCTVFCVLSGPGASCEAFSEGGIFNITSPMCLVSKQNRFRLTEQQVNFVCQRVDMDIVVYCNGQRKVILTKTLVIGQCIYTITSLFSLLPGVAHSIAVELCVPGFHGWATAALLVTFCFGWVLIPAITFIILTVLKFIANIFHTSNQENRLKSVLRKIKEEFEKTKGSMVCDVCKYECETYKELKAHGVSCPQSQCPYCFTHCEPTEAAFQAHYKVCQVTHRFRDDLKKTVTPQNFTPGCYRTLNLFRYKSRCYIFTMWIFLLVLESILWAASA

SEQ ID NO: 74 - protein, 487 aa, Hanta virus
SETPLTPVWNDNAHGVGSVPMHTDLELDFSLTSSSKYTYRRKLTNPLEEAQSIDLHIEIEEQTIGVDVHALGHWFDGRLNLKTSFHCYGACTKYEYPWHTAKCHYERDYQYETSWGCNPSDCPGVGTGCTACGLYLDQLKPVGSAYKIITIRYSRRVCVQFGEENLCKIIDMNDCFVSRHVKVCIIGTVSKFSQGDTLLFFGPLEGGGLIFKHWCTSTCQFGDPGDIMSPRDKGFLCPEFPGSFRKKCNFATTPICEYDGNMVSGYKKVMATIDSFQSFNTSTMHFTDERIEWKDPDGMLRDHINILVTKDIDFDNLGENPCKIGLQTSSIEGAWGSGVGFTLTCLVSLTECPTFLTSIKACDKAICYGAESVTLTRGQNTVKVSGKGGHSGSTFRCCHGEDCSQIGLHAAAPHLDKVNGISEIENSKVYDDGAPQCGIKCWFVKSGEWISGIFSGNWIVLIVLCVFLLFSLVLLSILCPVRKHKKS

SEQ ID NO: 75 - protein, 226 aa, Hepatitis B
MENITSGFLGPLLVLQAGFFLLTKILTIPQSLNSWWTSLSFLGGNTVCLGQNSQSPTSNHSPTSCPPTCPGYRWMCLRRFIIFLFILLLCLIFLLVLLDYQGMLPVCPLIPGSSTTSTGPCRTCKTPAQGTSMYPSCCCTKPSDGNCTCIPIPSSWAFGKFLWEWASARFSWLSLIVPFVQWFVGLSPTVWLSVIWMMWYWGPSLYSILSPFLPLLPIFFCLWVYI

SEQ ID NO: 76 - protein, 617 aa, Measles virus
MSPQRDRINAFYKDNPHPKGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPYLFNVPIKEAGEDCHAPTYLPAEVDGDVKLSSNLVILPGQDLQYVLATYDTSRVEHAVVYYVYSPSRSFSYFYPFRLPIKGVPIELQVECFTWDQKLWCRHFCVLADSESGGHITHSGMEGMGVSCTVTREDGTNRR

SEQ ID NO: 77 - protein, 550 aa, Measles virus
MGLKVNVSAIFMAVLLTLQTPTGQIHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILRSMKGLSSTSIVYILIAVCLGGLIGIPALICCCRGRCNKKGEQVGMSRPGLKPDLTGTSKSYVRSL

SEQ ID NO: 78 - protein, 500 aa, Zika virus
IRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFTCSKKMTGKSIQPENLEYRIMLSVHGSQHSGMIGYETDEDRAKVEVTPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLFSGHLKCRLKMDKLRLKGVSYSLCTAAFTFTKVPAETLHGTVTVEVQYAGTDGPCKIPVQMAVDMQTLTPVGRLITANPVITESTENSKMMLELDPPFGDSYIVIGVGDKKITHHWHRSGSTIGKAFEATVRGAKRMAVLGDTAWDFGSVGGVFNSLGKGIHQIFGAAFKSLFGGMSWFSQILIGTLLVWLGLNTKNGSISLTCLALGGVMIFLSTAVSA

SEQ ID NO: 79 - protein, 378 aa, Plasmodium vivax
MKNFILLAVSSILLVDLFPTHCGHNVDLSKAINLNGVNFNNVDASSLGAAHVGQSASRGRGLGENPDDEEGDAKKKKDGKKAEPKNPRENKLKQPGDRADGQPAGDRADGQPAGDRADGQPAGDRAAGQPAGDRADGQPAGDRADGQPAGDRADGQPAGDRADGQPAGDRAAGQPAGDRAAGQPAGDRADGQPAGDRAAGQPAGDRADGQPAGDRAAGQPAGDRADGQPAGDRAAGQPAGDRAAGQPAGDRAAGQPAGDRAAGQPAGNGAGGQAAGGNAGGGQGQNNEGANAPNEKSVKEYLDKVRATVGTEWTPCSVTCGVGVRVRRRVNAANKKPEDLTLNDLETDVCTMDKCAGIFNVVSNSLGLVILLVLALFN

SEQ ID NO: 80 - protein, 546 aa, Nipah virus
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 81 - protein, 602 aa, Nipah virus
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 82 - protein, 775 aa, Rotavirus A
MASLIYRQLLTNSYSVDLHDEIEQIGSEKTQNVTINPSPFAQTRYAPVNWGHGEINDSTTVEPILDGPYQPTTFTPPNDYWILINSNTNGVVYESTNNSDFWTAVVAIEPHVNPVDRQYTIFGESKQFNVSNDSNKWKFLEMFRSSSQNEFYNRRTLTSDTRFVGILKYGGRVWTFHGETPRATTDSSSTANLNNISITIHSEFYIIPRSQESKCNEYINNGLPPIQNTRNVVPLPLSSRSIQYKRAQVNEDIIVSKTSLWKEMQYNRDIIIRFKFGNSIVKMGGLGYKWSEISYKAANYQYNYLRDGEQVTAHTTCSVNGVNNFSYNGGSLPTDFGISRYEVIKENSYVYVDYWDDSKAFRNMVYVRSLAANLNSVKCTGGSYNFSIPVGAWPVMNGGAVSLHFAGVTLSTQFTDFVSLNSLRFRFSLTVDEPPFSILRTRTVNLYGLPAANPNNGNEYYEISGRFSLIYLVPTNDDYQTPIMNSVTVRQDLERQLTDLREEFNSLSQEIAMAQLIDLALLPLDMFSMFSGIKSTIDLTKSMATSVMKKFRKSKLATSISEMTNSLSDAASSASRNVSIRSNLSAISNWTNVSNDVSNVTNSLNDISTQTSTISKKFRLKEMITQTEGMSFDDISAAVLKTKIDMSTQIGKNTLPDIVTEASEKFIPKRSYRILKDDEVMEINTEGKFFAYKINTFDEVPFDVNKFAELVTDSPVISAIIDFKTLKNLNDNYGITRTEALNLIKSNPNMLRNFINQNNPIIRNRIEQLILQCKL

SEQ ID NO: 83 - protein, 230 aa, Rotavirus A
MASLIYRQLLTNSYSVDLHDEIEQIGSEKTQNVTINPSPFAQTRYAPVNWGHGEINDSTTVEPILDGPYQPTTFTPPNDYWILINSNTNGVVYESTNNSDFWTAVVAIEPHVNPVDRQYTIFGESKQFNVSNDSNKWKFLEMFRSSSQNEFYNRRTLTSDTRFVGILKYGGRVWTFHGETPRATTDSSSTANLNNISITIHSEFYIIPRSQESKCNEYINNGLPPIQNTR

SEQ ID NO: 84 - protein, 539 aa, Human metapneumovirus
MSWKVVIIFSLLITPQHGLKESYLEESCSTITEGYLSVLRTGWYTNVFTLEVGDVENLTCSDGPSLIKTELDLTKSALRELKTVSADQLAREEQIENPRQSRFVLGAIALGVATAAAVTAGVAIAKTIRLESEVTAIKNALKTTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGFIIVIILIAVLGSSMILVSIFIIIKKTKKPTGAPPELSGVTNNGFIPHS

SEQ ID NO: 85 - protein, 219 aa, Human metapneumovirus
MEVKVENIRAIDMLKARVKNRVARSKCFKNASLILIGITTLSIALNIYLIINYTIQKTSSESEHHTSSPPTESNKEASTISTDNPDINPNSQHPTQQSTENPTLNPAASVSPSETEPASTPDTTNRLSSVDRSTAQPSESRTKTKPTVHTRNNPSTASSTQSPPRATTKAIRRATTFRMSSTGKRPTTTSVQSDSSTTTQNHEETGSANPQASVSTMQN

SEQ ID NO: 86 - protein, 555 aa, Human parainfluenza virus
MQKSEILFLIYSSLLLSSSLCQIPVDKLSNVGVIINEGKLLKIAGSYESRYIVLSLVPSIDLEDGCGTTQIIQYKNLLNRLLIPLKDALDLQESLITITNDTTVTNDNPQSRFFGAVIGTIALGVATAAQITAGIALAEAREARKDIALIKDSIIKTHNSVELIQRGIGEQIIALKTLQDFVNNEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESRLAYICPRDPTQLIPDNQQKCILGDVSKCPVTKVINNLVPKFAFINGGVVANCIASTCTCGTNRIPVNQDRSRGVTFLTYTNCGLIGINGIELYANKRGRDTTWGNQIIKVGPAVSIRPVDISLNLASATNFLEESKIELMKAKAIISAVGGWHNTESTQIIIIIIVCILIIIICGILYYLYRVRRLLVMINSTHNSPVNTYTLESRMRNPYIGNNSN

SEQ ID NO: 87 - protein, 571 aa, Human parainfluenza virus
MEDYSNLSLKSIPKRTCRIIFRTATILGICTLIVLCSSILHEIIHLDVSSGLMDSDDSQQGIIQPIIESLKSLIALANQILYNVAIIIPLKIDSIETVIFSALKDMHTGSMSNTNCTPGNLLLHDAAYINGINKFLVLKSYNGTPKYGPLLNIPSFIPSATSPNGCTRIPSFSLIKTHWCYTHNVMLGDCLDFTTSNQYLAMGIIQQSAAAFPIFRTMKTIYLSDGINRKSCSVTAIPGGCVLYCYVATRSEKEDYATTDLAELRLAFYYYNDTFIERVISLPNTTGQWATINPAVGSGIYHLGFILFPVYGGLISGTPSYNKQSSRYFIPKHPNITCAGNSSEQAAAARSSYVIRYHSNRLIQSAVLICPLSDMHTARCNLVMFNNSQVMMGAEGRLYVIDNNLYYYQRSSSWWSASLFYRINTDFSKGIPPIIEAQWVPSYQVPRPGVMPCNATSFCPANCITGVYADVWPLNDPEPTSQNALNPNYRFAGAFLRNESNRTNPTFYTASASALLNTTGFNNTNHKAAYTSSTCFKNTGTQKIYCLIIIEMGSSLLGEFQIIPFLRELIP

SEQ ID NO: 88 - protein, 217 aa, Plasmodium falciparum
MNKLYSLFLFLFIQLSIKYNNAKVTVDTVCKRGFLIQMSGHLECKCENDLVLVNEETCEEKVLKCDEKTVNKPCGDFSKCIKIDGNPVSYACKCNLGYDMVNNVCIPNECKNVTCGNGKCILDTSNPVKTGVCSCNIGKVPNVQDQNKCSKDGETKCSLKCLKENETCKAVDGIYKCDCKDGFIIDNESSICTAFSAYNILNLSIMFILFSVCFFIM

SEQ ID NO: 89 - protein, 27 aa, synthetic construct
  REGION 1..27: note = T4 fibritin foldon domain
GYIPEAPRDGQAYVRKDGEWVLLSTFL

SEQ ID NO: 90 - protein, 540 aa, synthetic construct
  REGION 1..540: note = synthetically constructed Respiratory Synticial Virus antigen
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGYIPEAPRDGQAYVRKDGEWVLLSTFL

SEQ ID NO: 91 - protein, 646 aa, synthetic construct
  REGION 1..646: note = synthetically constructed Respiratory Synticial Virus antigen
MELLILKANVIATILTAVTFCFASSQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGYIPEAPRDGQAYVRKDGEWVLLSTFLGGSMEEVVLITVPSALVAVKIAHALVEERLAACVNIVPGLTSIYREEGSVVSDHELLLLVKTTTDAFPKLKERVKELHPYEVPEIVALPIAEGNREYLDWLRENTG

SEQ ID NO: 92 - protein, 619 aa, synthetic construct
  REGION 1..619: note = synthetically constructed Respiratory Synticial Virus antigen
MELLILKANVIATILTAVTFCFASSQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGGSMEEVVLITVPSALVAVKIAHALVEERLAACVNIVPGLTSIYREEGSVVSDHELLLLVKTTTDAFPKLKERVKELHPYEVPEIVALPIAEGNREYLDWLRENTG

SEQ ID NO: 93 - protein, 665 aa, synthetic construct
  REGION 1..665: note = synthetically constructed Respiratory Synticial Virus antigen
MELLILKANVIATILTAVTFCFASSQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGYIPEAPRDGQAYVRKDGEWVLLSTFLGGSMVRGIRGAITVNSDTPTSIIIATILLLEKMLEANGIQSYEELAAVIFTVTEDLTSAFPAEAARQIGMHRVPLLSAREVPVPGSLPRVIRVLALWNTDTPQDRVRHVYLSEAVRLRPDLESAQ

SEQ ID NO: 94 - protein, 638 aa, synthetic construct
  REGION 1..638: note = synthetically constructed Respiratory Synticial Virus antigen
MELLILKANVIATILTAVTFCFASSQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGGSMVRGIRGAITVNSDTPTSIIIATILLLEKMLEANGIQSYEELAAVIFTVTEDLTSAFPAEAARQIGMHRVPLLSAREVPVPGSLPRVIRVLALWNTDTPQDRVRHVYLSEAVRLRPDLESAQ

SEQ ID NO: 95 - protein, 769 aa, synthetic construct
  REGION 1..769: note = synthetically constructed Respiratory Synticial Virus antigen
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGYIPEAPRDGQAYVRKDGEWVLLSTFLGSGSHHHHHHHHGGSGGSGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 96 - protein, 730 aa, synthetic construct
  REGION 1..730: note = synthetically constructed Respiratory Synticial Virus antigen
MELLILKANVIATILTAVTFCFASSQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGGSGGSGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 97 - protein, 676 aa, synthetic construct
  REGION 1..676: note = synthetically constructed Respiratory Synticial Virus antigen
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGGSGGSGSDDARIAAIGDVDELNSQIGVLLAEPLPDDVRAALSAIQHDLFDLGGELCIPGHAAITEDHLLRLALWLVHYNGQLPPLEEFILPGGARGAALAHVCRTVCRRAERSIKALGASEPLNIAPAAYVNLLSDLLFVLARVLNRAAGGADVLWDRTRAH

SEQ ID NO: 98 - protein, 274 aa, Neisseria meningitidis
MNRTAFCCLSLTTALILTACSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 99 - protein, 370 aa, Neisseria meningitidis
MQTAARRSFDYDMPLIQTPTSACQIRQAWAKVADTPDRETAGRLKDEIKALLKETNAVLVAHYYVDPLIQDLALETGGCVGDSLEMARFGAEHEAGTLVVAGVRFMGESAKILCPEKTVLMPDLEAECSLDLGCPEEAFSAFCDQHPDRTVVVYANTSAAVKARADWVVTSSVALEIVSYLKSRGEKLIWGPDRHLGDYIRRETGADMLLWQGSCIVHNEFKGQELAALKAEHPDAVVLVHPESPQSVIELGDVVGSTSKLLKAAVSRPEKKFIVATDLGILHEMQKQAPDKQFIAAPTAGNGGSCKSCAFCPWMAMNSLGGIKYALTSGHNEILLDRKLGEAAKLPLQRMLDFAAGLKRGDVFNGMGPA

SEQ ID NO: 100 - protein, 492 aa, Neisseria meningitidis
MFKRSVIAMACIFALSACGGGGGGSPDVKSADTLSKPAAPVVSEKETEAKEDAPQAGSQGQGAPSAQGGQDMAAVSEENTGNGGAAATDKPKNEDEGAQNDMPQNAADTDSLTPNHTPASNMPAGNMENQAPDAGESEQPANQPDMANTADGMQGDDPSAGGENAGNTAAQGTNQAENNQTAGSQNPASSTNPSATNSGGDFGRTNVGNSVVIDGPSQNITLTHCKGDSCSGNNFLDEEVQLKSEFEKLSDADKISNYKKDGKNDGKNDKFVGLVADSVQMKGINQYIIFYKPKPTSFARFRRSARSRRSLPAEMPLIPVNQADTLIVDGEAVSLTGHSGNIFAPEGNYRYLTYGAEKLPGGSYALRVQGEPSKGEMLAGTAVYNGEVLHFHTENGRPSPSRGRFAAKVDFGSKSVDGIIDSGDGLHMGTQKFKAAIDGNGFKGTWTENGGGDVSGKFYGPAGEEVAGKYSYRPTDAEKGGFGVFAGKKEQD

SEQ ID NO: 101 - protein, 615 aa, synthetic construct
  REGION 1..615: note = synthetically constructed Respiratory Synticial Virus antigen
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGYIPEAPRDGQAYVRKDGEWVLLSTFLGGSMEEVVLITVPSALVAVKIAHALVEERLAACVNIVPGLTSIYREEGSVVSDHELLLLVKTTTDAFPKLKERVKELHPYEVPEIVALPIAEGNREYLDWLRENTG

SEQ ID NO: 102 - protein, 588 aa, synthetic construct
  REGION 1..588: note = synthetically constructed Respiratory Synticial Virus antigen
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGGSMEEVVLITVPSALVAVKIAHALVEERLAACVNIVPGLTSIYREEGSVVSDHELLLLVKTTTDAFPKLKERVKELHPYEVPEIVALPIAEGNREYLDWLRENTG

SEQ ID NO: 103 - protein, 634 aa, synthetic construct
  REGION 1..634: note = synthetically constructed Respiratory Synticial Virus antigen
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGYIPEAPRDGQAYVRKDGEWVLLSTFLGGSMVRGIRGAITVNSDTPTSIIIATILLLEKMLEANGIQSYEELAAVIFTVTEDLTSAFPAEAARQIGMHRVPLLSAREVPVPGSLPRVIRVLALWNTDTPQDRVRHVYLSEAVRLRPDLESAQ

SEQ ID NO: 104 - protein, 607 aa, synthetic construct
  REGION 1..607: note = synthetically constructed Respiratory Synticial Virus antigen
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGGSMVRGIRGAITVNSDTPTSIIIATILLLEKMLEANGIQSYEELAAVIFTVTEDLTSAFPAEAARQIGMHRVPLLSAREVPVPGSLPRVIRVLALWNTDTPQDRVRHVYLSEAVRLRPDLESAQ

SEQ ID NO: 105 - protein, 744 aa, synthetic construct
  REGION 1..744: note = synthetically constructed Respiratory Synticial Virus antigen
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGYIPEAPRDGQAYVRKDGEWVLLSTFLGSGSHHHHHHHHGGSGGSGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 106 - protein, 705 aa, synthetic construct
  REGION 1..705: note = synthetically constructed Respiratory Synticial Virus antigen
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGGSGGSGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 107 - protein, 645 aa, synthetic construct
  REGION 1..645: note = synthetically constructed Respiratory Synticial Virus antigen
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGGSGGSGSDDARIAAIGDVDELNSQIGVLLAEPLPDDVRAALSAIQHDLFDLGGELCIPGHAAITEDHLLRLALWLVHYNGQLPPLEEFILPGGARGAALAHVCRTVCRRAERSIKALGASEPLNIAPAAYVNLLSDLLFVLARVLNRAAGGADVLWDRTRAH

SEQ ID NO: 108 - protein, 678 aa, synthetic construct
  REGION 1..678: note = RSV F-10 DS-Cav1-8GS-HelExt-50A
QNITEEFYQSTCSAVSKGYLSALRTGWYTSVITIELSNIKENKCNGTDAKVKLIKQELDKYKNAVTELQLLMQSTPATNNRARRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGSGGSGSGEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 109 - protein, 682 aa, synthetic construct
  REGION 1..682: note = RSV F-11 DS-Cav1-12GS-HelExt-50A
QNITEEFYQSTCSAVSKGYLSALRTGWYTSVITIELSNIKENKCNGTDAKVKLIKQELDKYKNAVTELQLLMQSTPATNNRARRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 110 - protein, 686 aa, synthetic construct
  REGION 1..686: note = RSV F-12 DS-Cav1-16GS-HelExt-50A
QNITEEFYQSTCSAVSKGYLSALRTGWYTSVITIELSNIKENKCNGTDAKVKLIKQELDKYKNAVTELQLLMQSTPATNNRARRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGSGGSGSGSGGSGSGGEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 111 - protein, 680 aa, synthetic construct
  REGION 1..680: note = RSV F-13 DS-Cav1-foldon-10GS-HelExt-50A
QNITEEFYQSTCSAVSKGYLSALRTGWYTSVITIELSNIKENKCNGTDAKVKLIKQELDKYKNAVTELQLLMQSTPATNNRARRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGSGGSGSGSGEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 112 - protein, 712 aa, synthetic construct
  REGION 1..712: note = RSV_F-14 DS-Cav1-foldon-15GS-HelExt-50A
QNITEEFYQSTCSAVSKGYLSALRTGWYTSVITIELSNIKENKCNGTDAKVKLIKQELDKYKNAVTELQLLMQSTPATNNRARRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGYIPEAPRDGQAYVRKDGEWVLLSTFLGSGGSGSGSGGSGSGEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 113 - protein, 717 aa, synthetic construct
  REGION 1..717: note = RSV F-15 DS-Cav1-foldon-20GS-HelExt-50A
QNITEEFYQSTCSAVSKGYLSALRTGWYTSVITIELSNIKENKCNGTDAKVKLIKQELDKYKNAVTELQLLMQSTPATNNRARRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRGYIPEAPRDGQAYVRKDGEWVLLSTFLGSGGSGSGSGGSGSGGSSGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

(19) United States
(12) Patent Application Publication
(10) Pub. No.: US 2024/0165218 A1
(43) Pub. Date: May 23, 2024

(54) SELF-ASSSEMBLING NANOSTRUCTURE VACCINES

(71) Applicant: University of Washington, Seattle, WA (US)

(72) Inventors: Neil King, Seattle, WA (US); David Baker, Seattle, WA (US); Lance Stewart, Seattle, WA (US); Brooke Fiala, Seattle, WA (US); Daniel Ellis, Seattle, WA (US); Lauren Carter, Seattle, WA (US); Rashmi Ravichandran, Seattle, WA (US); George Ueda, Seattle, WA (US); Jorge Fallas, Seattle, WA (US); Una Natterman, Seattle, WA (US)

(21) Appl. No.: 18/359,154

(22) Filed: Jul. 26, 2023

Related U.S. Application Data
(63) Continuation of application No. 16/971,278, filed on Aug. 19, 2020, now Pat. No. 11,771,755.
(60) Provisional application No. 62/724,721, filed on Aug. 30, 2018.
(60) Provisional application No. 62/636,757, filed on Feb. 28, 2018.

(51) Int. Cl. A61K 39/12; C12N 15/86

(52) U.S. Cl. CPC A61K 39/12; C12N 15/86; B82Y 5/00

(57) ABSTRACT

The present disclosure provides nanostructures and nanostructure-based vaccines. Some nanostructures of the present disclosure display antigens capable of eliciting immune responses to infectious agents such as bacteria, viruses, and pathogens. Some vaccines of the present disclosure are useful for preventing or decreasing the severity of infection with an infectious agent, including. for example and without limitation, lyme disease, pertussis, herpes virus, orthomyxovirus, paramyxovirus, pneumovirus, filovirus. flavivirus, reovirus, retrovirus, meningococcus, or malaria. The antigens may be attached to the core of the nanostructure either non-covalently or covalently, including as a fusion protein or by other means disclosed herein. Multimeric antigens may optionally be displayed along a symmetry axis of the nanostructure. Also provided are proteins and nucleic acid molecules encoding such proteins. vaccine compositions, and methods of administration.

### CROSS REFERENCE

This application claims priority to U.S. Provisional Patent Application Ser. No. 62/636,757 filed Feb. 28, 2018 and U.S. Provisional Patent Application Ser. No. 62/724,721 filed Aug. 30, 2018, each incorporated by reference herein in its entirety.

### DESCRIPTION OF THE TEXT FILE SUBMITTED ELECTRONICALLY

This application is being filed electronically via EFS-Web and includes an electronically submitted sequence listing in .txt format. The .txt file contains a sequence listing entitled “ICVX_001_02WO_SeqList_ST25.txt” created on Feb. 27, 2019 and having a size of ˜373 kilobytes.

### FIELD OF THE INVENTION

The present disclosure relates generally to vaccines and methods of use thereof. Specifically, the disclosure relates to nanostructure-based vaccines capable of eliciting immune responses to antigens, such as antigenic proteins of various infectious agents, including bacteria, viruses, and parasites.

### BACKGROUND OF THE INVENTION

Vaccination is a treatment modality used to prevent or decrease the severity of infection with various infectious agents, including bacteria, viruses, and parasites. Development of new vaccines has important commercial and public health implications. In particular, lyme disease, pertussis, herpes virus, orthomyxovirus, paramyxovirus. pneumovirus, filovirus, flavivirus, reovirus, retrovirus, and malaria are infectious agents for which vaccines already exist, are being developed, or would be desirable.

Subunit vaccines are vaccines made from isolated antigens, usually proteins expressed recombinantly in bacterial, insect, or mammalian cell hosts. Typically, the antigenic component of a subunit vaccine is selected from among the proteins of an infectious agent observed to elicit a natural immune response upon infection, although in some cases other components of the infectious agent can be used. Typical antigens for use in subunit vaccines include protein expressed on the surface of the target infectious agent, as such surface-expressed envelope glycoproteins of viruses. Preferably, the antigen is a target for neutralizing antibodies. More preferably, the antigen is a target for broadly neutralizing antibodies, such that the immune response to the antigen covers immunity against multiple strains of the infectious agent. In some cases, glycans that are N-linked or O-linked to the subunit vaccine may also be important in vaccination, either by contributing to the epitope of the antigen or by guiding the immune response to particular epitopes on the antigen by steric hindrance. The immune response that occurs in response to vaccination may be direct to the protein itself, to the glycan, or to both the protein and linked glycans. Subunit vaccines have various advantages including that they contain no live pathogen, which eliminates concerns about infection of the patient by the vaccine; they may be designed using standard genetic engineering techniques: they are more homogenous than other forms of vaccine: and they can be manufactured in standardized recombinant protein expression production systems using well-characterized expression systems. In some cases, the antigen may be genetically engineered to favor generation of desirable antibodies, such as neutralizing or broadly neutralizing antibodies. In particular, structural information about an antigen of interest, obtained by X-ray crystallography, electron microscopy, or nuclear magnetic resonance experiments, can be used to guide rational design of subunit vaccines.

A known limitation of subunit vaccines is that the immune response elicited may sometimes be weaker than the immune response to other types of vaccines, such as whole virus, live, or live attenuated vaccines. The present inventors have recognized and herein disclose that nanostructure-based vaccines have the potential to harness the advantages of subunit vaccines while increasing the potency and breadth of the vaccine-induced immune response through multivalent display of the antigen in symmetrically ordered arrays. Nanostructure-based vaccines are one form of “nanoparticle vaccine.” In the present disclosure, nanostructure-based vaccines are distinguished from nanoparticle vaccines. because the term nanoparticle vaccine has been used in the art to refer to protein-based or glycoprotein-based vaccines (see, e.g. U.S. Patent No. US 9,441,019), polymerized liposomes (see, e.g., U.S. Pat. No. 7,285,289), surfactant micelles (see, e.g., US Patent Pub. No. US 2004/0038406 A1), and synthetic biodegradable particles (see, e.g., U.S. Pat. No. 8,323,696). Nanostructure-based vaccination represents a paradigm in vaccination with significant commercial and public health implications. Thus, there exists a need for nanostructure-based vaccines and methods of use thereof for eliciting immune responses to infectious agents, such as bacteria, viruses, and parasites; and for preventing or decreasing the severity of infection with an infectious agent including, for example and without limitation, lyme disease, pertussis, herpes virus, orthomyxovirus, paramyxovirus, pneumovirus, filovirus, flavivirus, reovirus, retrovirus, meningococcus, and malaria.

### SUMMARY OF THE INVENTION

Described herein are nanostructures, vaccines, methods of use thereof, and methods of making said nanostructures.

In one aspect, the present disclosure provides nanostructures comprising a first plurality of polypeptides, wherein the first plurality of polypeptides are arranged according to at least one symmetry operator; the nanostructure comprises a first plurality of antigens: each of the first plurality of the antigens has a proximal end and a distal end; and the proximal ends of the antigens are each attached to a member of the first plurality of polypeptides.

In another aspect, the present disclosure provides vaccines comprising any of the nanostructures of the present disclosure, wherein the vaccine is capable of eliciting a neutralizing antibody response to an infectious agent. In an embodiment, the vaccine is provided in a pharmaceutical composition.

In another aspect, the present disclosure provides methods of generating immunity to an infectious agent in a subject, comprising administering any of the vaccines of the present disclosure.

In another aspect, the present disclosure provides methods of making any of the nanostructures of the present disclosure by in vitro assembly of component purified from one or more recombinant expression systems. In another aspect, the present disclosure provides methods of making any of the nanostructures of the present disclosure by co-expression of all components in a recombinant expression system, thereby generating the nanostructure, and purifying the nanostructure.

In an embodiment of the nanostructures of the present disclosure, the nanostructure further comprises a second plurality of polypeptides, wherein the second plurality of polypeptides is attached to the first plurality of polypeptides. In an embodiment, the nanostructure further comprises a second plurality of antigens. In an embodiment, the nanostructure further comprises a second plurality of antigens, each of the second plurality of second antigens has a proximal end and a distal end, and the proximal ends of the second antigens are each attached to a member of the second plurality of polypeptides; and optionally, the proximal ends of the antigens are the N termini of the antigens or the C termini of the antigens.

In an embodiment of the nanostructures of the present disclosure, the plurality of antigens is a plurality of antigenic proteins or antigenic fragments thereof. In an embodiment, the antigenic protein of the nanostructure is selected from SEQ ID NOs: 52-88 and 90-113 or a variant thereof; or the antigenic protein is at least 75, 80, 85, 90, 95, or 99% identical to a polypeptide selected from SEQ ID NOs: 52-88 and 90-97; or the antigenic protein is any of the following: HIV Env, RSV F, Influenza HA, EBV gp350, CMV gB, CMV UL128, CMV UL130, CMV UL131A, CMV gH, CMV gL, Lyme OspA. Pertussis toxin, Dengue E. SARS S, MERS S, Zaire ebolavirus GP, Sudan ebolavirus GP, Marburg virus GP, Hanta virus Gn, Hanta virus Gc, HepB surface antigen, Measles H, Zika envelope domain III, Malaria CSP, Malaria Pfs25, MenB fHbp, MenB NadA, MenB NHBA, Nipah virus F, Nipah virus G. Rotavirus VP4, Rotavirus VP8*, hMPV F, hMPV G, PV F, or PV HN 8.

In an embodiment, the nanostructure is configured to display a target epitope of the antigen; and optionally, the target epitope is accessible to an antibody as defined herein below. In any embodiment where the nanostructure comprises a plurality of antigenic proteins, optionally the nanostructure is configured to elicit an immune response to the first plurality of antigenic proteins, which immune response is preferentially directed to a target epitope of the antigenic protein. In embodiments of the present disclosure, the target epitope is conserved, it is an epitope for neutralizing antibodies, it is an epitope for cross-reactive antibodies, or it is an epitope for a broadly-neutralizing antibody.

In an embodiment of the nanostructures of the present disclosure, the plurality of antigens comprises at least one mutation selected from the group consisting of an interface-stabilizing mutation, complementary cysteine mutations configured to result in a disulfide bond, deletion of a loop, addition of an N-linked glycosylation site, removal of an N-linked glycosylation site, an epitope-destroying mutation, and an epitope-creating mutation. In an embodiment, the plurality of antigens comprises an antigenic oligosaccharide.

In an embodiment of the vaccines of the present disclosure, the neutralizing antibody response is protective against infection by an infectious agent. In an embodiment. the neutralizing antibody response is broadly-neutralizing against diverse strains of an infectious agent. In an embodiment, the infectious agent is any of the following: lyme disease, pertussis, herpesvirus, orthomyxovirus, paramyxovirus, pneumovirus, filovirus, flavivirus, reovirus, retrovirus, meningococcus, or malaria. In an embodiment, the infectious agent is a virus selected from the following: HIV. RSV. Influenza, EBV, CMV, Dengue, Severe Acute Respiratory Syndrome (SARS) virus, Middle East Respiratory Syndrome (MERS) virus, Ebola virus, Marburg virus, Hanta virus, Hepatitis B, HPV, Measles, Nipah virus, Rotavirus, Metapneumo virus, Parainfluenza virus, and Zika. In an embodiment, the infectious agent is lyme disease or pertussis. In an embodiment, the infectious agent is malaria. In an embodiment, the infectious agent is meningococcus.

In an embodiment of the methods of generating immunity of the present disclosure, the method further comprises administering an adjuvant. In an embodiment, the method further comprises administering the vaccine repeatedly. In an embodiment, the method further comprises administering a second vaccine which is selected from following: a nanoparticle-based vaccine, a protein-based vaccine, a live vaccine, a live attenuated vaccine, a whole germ vaccine, a DNA vaccine, or a RNA vaccine: and optionally the first vaccine is a prime and the second vaccine is a boost, or optionally the second vaccine is a prime and the first vaccine is a boost. In an embodiment, the method induces directed affinity maturation. In an embodiment, the method results in a broadly-neutralizing immune response.

In an embodiment of the methods of making any of the nanostructures of the present disclosure, the method achieves in vitro assembly of the nanostructure by sequentially or non-sequentially expressing the first plurality of polypeptides in a first recombinant expression system, expressing the first plurality of antigens in a second recombinant expression system, purifying the first plurality of polypeptides, purifying the first plurality of antigens, provided that expression of each component precedes purification of that component; and then mixing the first plurality of polypeptides and the first plurality of antigens: thereby generating the nanostructure.

In an embodiment of the methods of making a nanostructure, the method achieves in vitro assembly of the nanostructure by sequentially or non-sequentially expressing the first plurality of polypeptides in a first recombinant expression system, expressing the first plurality of antigens in a second recombinant expression system, expressing the second plurality of polypeptides in a third recombinant expression system, purifying the first plurality of polypeptides, purifying the first plurality of antigens, purifying the second plurality of polypeptides, provided that expression of each component precedes purification of that component; and mixing the first plurality of polypeptides, the first plurality of antigens, and the second plurality of polypeptides; thereby generating the nanostructure. Optionally, the first recombinant expression system and the second recombinant expression system are the same, and the first plurality of polypeptides and the first plurality of antigens are purified together.

In an embodiment of the methods of making a nanostructure, the method comprises expressing the first plurality of polypeptides and the first plurality of antigens in a single recombinant expression system, thereby generating the nanostructure, and purifying the nanostructure. In an embodiment, the method comprises expressing the first plurality of polypeptides, the first plurality of antigens, and the second plurality of polypeptides in a single recombinant expression system, thereby generating the nanostructure, and purifying the nanostructure. In an embodiment, optionally, the first plurality of polypeptides and the first plurality of antigens are encoded by a single open reading frame: and optionally, the single open reading frame encodes a fusion protein of the polypeptide and the antigen: and optionally, the single open reading frame encodes a self-cleaving peptide.

The foregoing paragraphs are not intended to define every aspect of the invention, and additional aspects are described in other sections, such as the Detailed Description. The entire document is intended to be related as a unified disclosure, and it should be understood that all combinations of features described herein are contemplated, even if the combination of features are not found together in the same sentence, or paragraph, or section of this document. The invention includes, as an additional aspect, all embodiments of the invention narrower in scope in any way than the variations defined by specific paragraphs above. For example, where certain aspects of the invention that are described as a genus, it should be understood that every member of a genus is, individually, an aspect of the invention.

### BRIEF DESCRIPTION OF THE DRAWINGS

FIG. 1A shows a schematic diagram of the production of antigen-bearing nanostructures by in vitro assembly. The two components or building blocks of a given nanostructure can be expressed and purified individually, which allows assembly of the nanostructure to be initiated by mixing the purified components in vitro, a process referred to as in vitro assembly. In some embodiments, the two components of the nanostructure may be expressed in different expression hosts (e.g., human HEK293F cells or bacterial E. coli cells). The figure schematically depicts assembly of a 120-subunit nanostructure bearing 20 trimeric antigens (60 antigen subunits) via in vitro assembly of an antigen-nanostructure trimer fusion protein produced in HEK293F cells and a nanostructure pentamer protein produced in E. coli

FIG. 1B depicts example nanostructure architectures.

FIGS. 2A-2C shows graphs illustrating detection of secreted DS-Cav1 (FIG. 2A), DS-Cav1-foldon-T33-31A (FIG. 2B), and DS-Cav1-T33-31A (FIG. 2C) fusion proteins in tissue culture supernatants. ELISA assays were performed on tissue culture supernatants from cells expressing DS-Cav1 (top), DS-Cav-1-foldon-T33-31A/T33-31B (bottom left), and DS-Cav-1-T33-31A/T33-31B (bottom right). Four different monoclonal antibodies that bind RSV F were used to evaluate the presence of DS-Cav1 or DS-Cav1 fusion proteins in the superatants. The results confirm the secretion of proteins comprising well-folded RSV F antigen.

FIG. 3 shows size-exclusion chromatography of DS-Cav1-I53-50A. Protein purified from tissue culture supernatants by immobilized metal affinity chromatography was applied to a Superose 6 10/300 GL size exclusion column. The protein eluted as a single, monodisperse species.

FIG. 4 shows size exclusion chromatography of in vitro-assembled DS-Cav1-I53-50 nanostructures. Purified DS-Cav1-I53-50A and I53-50B.4PT1 proteins were mixed at an approximately 1:1 molar ratio, incubated overnight at 4° C., and then applied to a Sephacryl S-500 16/60 HR size exclusion column. The assembled nanostructure eluted as a single, monodisperse peak around 65 mL, while excess DS-Cav1-I53-50A trimeric component eluted around 90 mL.

FIG. 5 shows a negative stain electron micrograph and two-dimensional class averages of in vitro-assembled DS-Cav1-I53-50 nanostructures. In vitro-assembled DS-Cav1-I53-50 nanostructures, purified by size exclusion chromatography, were imaged by negative stain electron microscopy (top). Averaging many nanostructures yielded two-dimensional class averages (bottom) that indicate that the I53-50 portion of the nanostructures is highly ordered and consistent, while the precise three-dimensional position of the displayed antigen varies slightly due to the flexible nature of the linker between the DS-Cav1 and I53-50A domains of the DS-Cav1-I53-50A fusion protein.

FIGS. 6A-6C shows a series of graphs depicting the antigenicity of DS-Cav1-I53-50 nanostructures. Analysis of purified DS-Cav1-I53-50 nanostructures by ELISA (FIG. 6A) using four RSV F-specific monoclonal antibodies, including the prefusion-specific antibodies MPE8. D25, and RSD5. indicated that the DS-Cav1 antigen is correctly folded and maintained in the prefusion state when multivalently displayed on DS-Cav1-I53-50 nanostructures. This finding was confirmed by surface plasmon resonance measurements using multiple RSV F-specific antibodies, which, when compared to trimeric DS-Cav1 (FIG. 6C), further suggested that multivalent display of DS-Cav1 (FIG. 6B) results in an avidity effect that reduces the dissociation rate of the antibodies.

FIG. 7 is a graph depicting DS-Cav1-specific serum antibody titers from mice immunized with DS-Cav1-I53-50 nanostructures. Groups of mice were immunized with I53-50 nanostructures lacking additional antigen, trimeric DS-Cav1, or I53-50 nanostructures bearing DS-Cav1 antigen at 33%, 66%, or 100% valency. DS-Cav1-specific serum antibody titers were measured by ELISA on plates coated with DS-Cav1. Serum antibody titers for each mouse are plotted as circles, with the geometric mean within each group plotted as a horizontal line and reported numerically at bottom.

FIG. 8 is a graph depicting serum neutralization activity elicited by immunization with DS-Cav1-I53-50 nanostructures. Groups of mice were immunized with I53-50 nanostructures lacking additional antigen, trimeric DS-Cav1, or I53-50 nanostructures bearing DS-Cav1 antigen at 33%, 66%, or 100% valency. Neutralization titers for each mouse are plotted as circles, with the geometric mean within each group plotted as a horizontal line.

FIGS. 9A-9B are graphs depicting immunogenicity in a primate immune system elicited by immunization with DS-Cav1-foldon I53-50 nanostructures. Rhesus macaques were injected with DS-Cav1-foldon-I53-50 nanostructures intramuscularly at weeks 0 and 4 with either free DS-Cav1 trimer or DS-Cav1-foldon-I53-50 nanostructures displaying DS-Cav1 at 100% valency. In both cases, the dose of DS-Cav1 antigen was 50 μg, and the immunogens were formulated with the MF59-like, squalene-based oil-in-water emulsion adjuvant SWE. Sera obtained from the animals at weeks 6 and 16 were evaluated for anti-DS-Cav1 antibody titers (FIG. 9A) and RSV-neutralizing antibody titers (FIG. 9A).

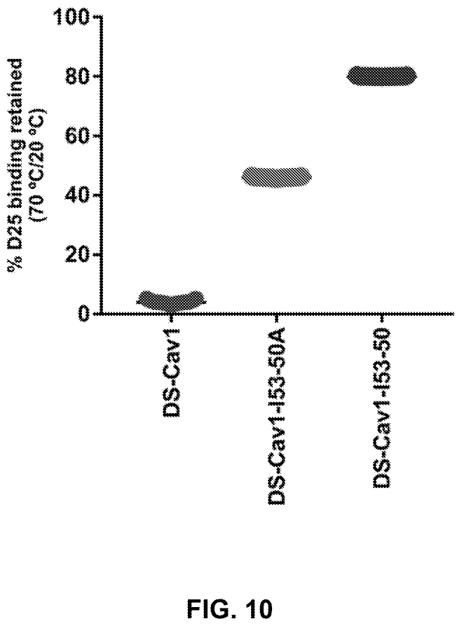

FIG. 10 is a graph depicting the physical stability of DS-Cav1 when fused to I53-50A and/or when further assembled into the icosahedral nanostructure. Samples of trimeric DS-Cav1, trimeric DS-Cav1-foldon-I53-50A, and DS-Cav1-foldon-I53-50 nanostructures containing equivalent concentrations (50 nM) of DS-Cav1 were split into four aliquots and incubated at 20, 50, 70 or 80° C. for 1 hour. After cooling to room temperature, D25 binding was assayed by surface plasmon resonance (SPR).

FIGS. 11A-11J are graphs depicting physical stability of the nanostructures. Chemical denaturation in guanidine hydrochloride (GdnHCl), monitored by intrinsic tryptophan fluorescence, was used as a second, antibody-independent technique to evaluate physical stability of trimeric DS-Cav1 (FIG. 1A and FIG. 1B), DS-Cav1-foldon-I53-50A (FIG. 1C and FIG. 1D), DS-Cav1-foldon-I53-50 (FIG. 1E and FIG. 1F), I53-50 (FIG. 1G and FIG. 1H), and I53-50A (FIG. 1I and FIG. 11J). The data indicate superior physical stability of the DS-Cav1 antigen when genetically fused to the I53-50A nanostructure component.

### DETAILED DESCRIPTION

The present disclosure relates to nanostructures and nanostructure-based vaccines. Some nanostructures of the present disclosure display antigens capable of eliciting immune responses to infectious agents, such as bacteria, viruses, and parasites. Some vaccines of the present disclosure are useful for preventing or decreasing the severity of infection with an infectious agent including, for example and without limitation, lyme disease, pertussis, herpes virus, orthomyxovirus, paramyxovirus, pneumovirus, filovirus, flavivirus, reovirus. retrovirus, meningococcus, and malaria. The antigens may be attached to the core of the nanostructure either non-covalently or covalently, including as a fusion protein or by other means disclosed herein. Multimeric antigens may optionally be displayed along a symmetry axis of the nanostructure. Also provided are proteins and nucleic acid molecules encoding such proteins, formulations, and methods of use.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the claims.

### 1. Overview of Nanostructures

The nanostructures of the present invention may comprise multimeric protein assemblies adapted for display of antigens or antigenic fragments. The nanostructures of the present invention comprise at least a first plurality of polypeptides. The first plurality of polypeptides may be derived from a naturally-occurring protein sequence by substitution of at least one amino acid residue or by additional at the N- or C-terminus of one or more residues. In some cases, the first plurality of polypeptides comprises a gene sequence determined de novo by computational methods. This first plurality of polypeptides may form the entire nanostructure; or the nanostructure may comprise one or more additional polypeptides, such that the nanostructure comprises two, three, four, five, six, seven, or more pluralities of polypeptides. In some cases, the first plurality will form trimers related by 3-fold rotational symmetry and the second plurality will form pentamers related by 5-fold rotational symmetry. Together these one or more pluralities of polypeptides may be arranged such that the members of each plurality of polypeptides are related to one another by symmetry operators. A general computational method for designing self-assembling protein materials, involving symmetrical docking of protein building blocks in a target symmetric architecture, is disclosed in U.S. Patent Pub. No. US 2015/0356240 A1.

The “core” of the nanostructure is used herein to describe the central portion of the nanostructure that links together the antigens or antigenic fragments displayed by the nanostructure. In an embodiment, the core and the displayed antigens are the same polypeptide, meaning that antigens are themselves capable of self-assembly into a nanostructure. An advantage of designing the antigens themselves to self-assemble is that the entire nanostructure then acts as the antigenic component of the vaccine. But in an embodiment, the cores of the nanostructures of the present disclosure are generic platforms adaptable for display of any of various antigens that one might select for inclusion in a vaccine. An advantage of designing a core to be a generic platform is that the one or more pluralities of polypeptides that comprise the core can be designed and optimized in advance and then applied to different antigens. It will be understood that in some cases, the same polypeptide may form a portion of the “core” and then extend outward as either an adaptor for attachment of an antigen and as the antigen itself (i.e., a fusion protein with the antigen). In embodiments of the present disclosure, the antigen is a protein, glycoprotein, or oligosaccharide of an infectious agent.

In some cases, self-assembly may be further promoted by multimerization of the antigen even though the core would, in absence of the antigen, be independently capable of self-assembly. This would be the case for example when a homo-trimeric antigen (such as HIV gp140, influenza HA, or RSV F protein) is the antigen, or one of several antigens, displayed on the particle. In some cases, a trimeric antigen placed along a 3-fold axis of the nanostructure promotes proper folding and conformation stability of the antigen and makes self-assembly of the nanostructure a cooperative process, in that the antigen is trimerized properly in part due to its display on a 3-fold axis of the core of the nanostructure, and the nanostructure is stabilized in its assembled form, at least in part, by non-covalent or covalent interactions amongst the trimer units. In some cases, introduction of mutations to the antigen or to the nanostructure components may optionally further stabilize assembly, in particular if cysteine residues are position to create intramolecular disulfide bonds. In some examples, a dimeric, trimeric, tetrameric, pentameric, or hexameric antigen is displayed upon a core designed to have a matching 2-fold, 3-fold, 4-fold, 5-fold, or 6-fold symmetry axis such that the core accommodates the arrangement of the multimeric antigen with the native symmetry of the antigen.

### 2. Various Non-Limiting Examples of Nanostructures

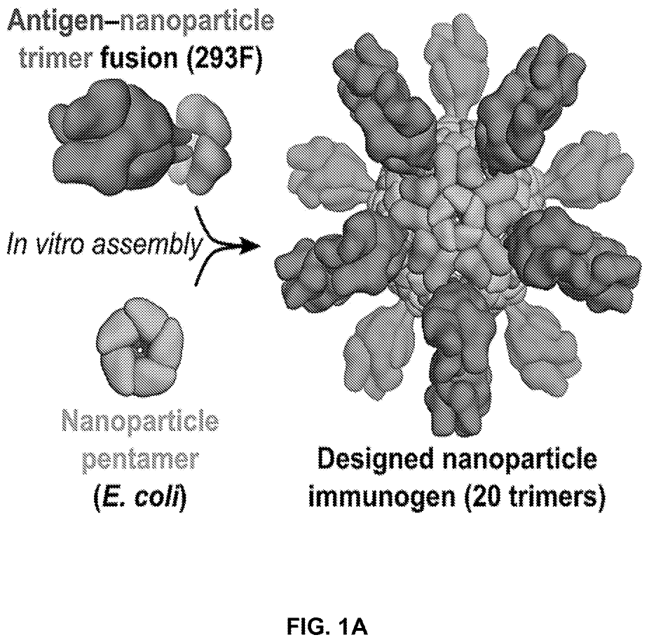

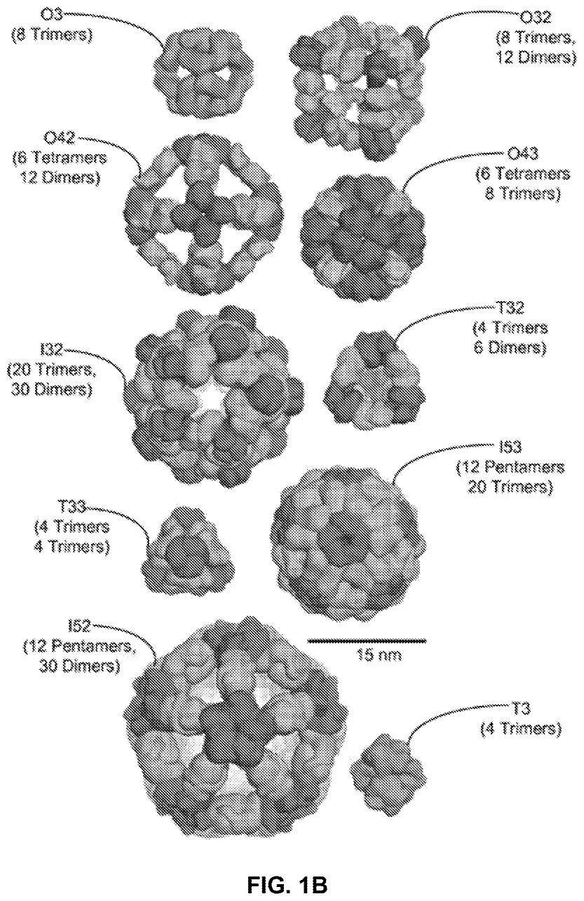

A non-limiting example of an embodiment is shown in FIG. 1A, which depicts the RSV F protein genetically fused to a component (a first plurality of polypeptides) of the nanostructure, which is expressed recombinantly in 293F cells: along with a pentameric protein assembly (a second plurality of polypeptides), which is expressed recombinantly in E. coli cells, these two pluralities of polypeptides self-assembling into a nanostructure (a “designed nanoparticle immunogen”) displaying 20 F-protein trimers around an icosahedral core. In this embodiment, the core has a generic design. As explained below, in other embodiments, the RSV F protein is replaced with other another antigen protein, such as a trimeric glycoprotein from another virus. In some embodiments, the nanostructure comprises the trimeric glycoproteins of HIV-1, HIV-2, EBV, CMV, RSV, influenza, Ebola, Marburg, Dengue. SARS, MERS. Hantaan, or Zika virus. In some embodiments, the nanostructure comprises the trimeric glycoproteins of viruses that are related evolutionarily or in sequence identity to any of these exemplary virus, including without limitation, a herpes virus, orthomyxovirus, paramyxovirus, pneumovirus, filovirus, flavivirus, reovirus, or retrovirus. In an embodiment, the nanostructure comprises the extracellular domain or domains of a transmembrane protein or glycoprotein, or an antigenic fragment thereof. In some embodiments, the nanostructure comprises the antigen proteins or protein fragments or antigenic oligosaccharides of a bacterial pathogen, including without limitation, Neisseria meningitides (also known as “meningococcus”), Haemophilus influenzae type B, Streptococcus pneumonia, and Listeria monocytogenes.

Trimeric antigens that may be used with this or similar nanostructures are in some cases, without limitation, HIV gp140, influenza HA, dengue E protein, or Ebola sGP. When other trimeric antigens are used, they may optionally be placed on the 3-fold symmetry axis of the nanostructure. In some cases, the antigen chosen is monomeric and nevertheless placed on a 3-fold axis. Thus, the nanostructure depicted in FIG. 1A is capable of displaying 20 timeric antigens or 60 monomeric antigens. Additionally or alternatively the pentameric complexes of the nanostructure is used to display a 12 pentameric antigens or 70 monomeric antigens. In an embodiment, the nanostructure comprises 20 copies of a trimeric antigen and 12 copies of a pentameric antigen.

### 2.1. Nanostructure Cores

Other potential arrangements of polypeptides of the present disclosure are shown in FIG. 1B. In some embodiments, the nanostructure is adapted for display of up to 8 trimers: 8 timers and 12 dimers: 6 tetramers and 12 dimers; 6 tetramers and 8 trimers; 20 trimers and 30 dimers: 4 trimers and 6 dimers; 4 first trimers and 4 second trimers, or 8 trimers: 12 pentamers and 20 trimers; or 12 pentamers and 30 dimers: or 4 trimers. In some cases, one of the symmetric axes is not used for antigen display, thus, in some embodiments the nanostructure is adapted for display of up to 8 trimers: 12 dimers; 6 tetramers: 20 trimers: 30 dimers; 4 trimers: 6 dimers: 8 trimers; or 12 pentamers. In some cases, monomeric antigens are displayed and thus, the nanostructure is adapted for display of up to 12, 24, 60, or 70 monomeric antigens. In some cases, the nanostructure comprises mixed pluralities of polypeptides such that otherwise identical polypeptides of the core of the nanostructure display different antigens or no antigen. Thus, depending on the ratio of polypeptides, the nanostructure is in some cases adapted for display of between I and 130 antigens (e.g., on the I52 particle) where each of the antigens displayed may be the same or may be different members of mixed population in proportion to any ratio chosen. The antigens may be co-expressed in a recombinant expression system and self-assembled before purification. Alternatively, the antigens may be expressed separately and then mixed together, either before or after purification from expression host and associated contaminants. In various embodiments, 1, 2, 3, 4, 5, 6, 7, 8, 9, 10, 11, 12, 13, 14, 15, 16, 17, 18, 19, 20, 21, 22, 23, 24, 25, 26, 27, 28, 29. 30, or more antigens are displayed. Non-limiting exemplary nanostructures are provided in Bale et al. Science 353:389-94 (2016); Heinze et al. J. Phys. Chem B. 120:5945-5952 (2016): King et al. Nature 510:103-108 (2014): and King et al. Science 336:1171-71 (2012).

### 2.2. Mixed Nanostructures

In some embodiments, the nanostructure displays two or more antigens from the same organism, such as without limitation HIV gp140 and HIV gp41; or Ebola virus GP1 and GP2: or Measles H and F proteins: or CMV gB and CMV UL128, UL130, UL131A, gH (UL75) and gL (UL115), on the same nanostructure. In some cases, the nanostructure displays two antigenic proteins or glycoproteins that are generated by post-transcriptional cleavage, such as cleavage of RSV F protein or influenza HA protein by proteases endogenous to the recombinant expression system, or by proteases supplied exogenously.

In some cases, the nanostructure is adapted to display the same antigen from two or more diverse strain of a pathogenic organism. In non-limiting examples, the same nanostructure displays mixed populations of homotrimeric protein antigens or mixed heterotrimers of protein antigens from different strains of the infectious agent. In an embodiment, the nanostructure displays the HA proteins of an H1N1 influenza A and of an H3N2 influenza A proteins. In an embodiment, the nanostructure displays the HA proteins of an influenza A and of an influenza B. In an embodiment, the gp 140 proteins from diverse strains of HIV are displayed on a single nanostructure. Two, three, four, five, or six strains of HIV may be displayed by the same nanostructure. Without being bound by theory, an advantage of such a mixed nanostructure is that it promotes the generation of cross-reactive or broadly neutralizing immune responses. In some cases, the nanostructure-based vaccine of the present disclosure is a universal influenza vaccine. In some cases, the nanostructure-based vaccine of the present disclosure is an HIV vaccine. In some case, the nanostructure-based vaccine of the present disclosure provides enduring protection against HIV. In some case, the nanostructure-based vaccine of the present disclosure provides enduring protection against influenza. In an embodiment, the nanostructure is adapted for display of the E proteins of Dengue type 1, type 2, type 3, and type 4. In an embodiment, the nanostructure-based vaccine comprises nanostructures that individually display the protein E from each of Dengue type 1, type 2, type 3, and type 4. In an embodiment, the nanostructure-based vaccine of the present disclosure provides immunity to Dengue virus without increased risk of dengue hemorrhagic fever or dengue shock syndrome.

When mixed nanostructures are made, it may be advantageous to ensure homomerization in a strain-specific manner rather than permit heterodimerization, such that, for example all H1N1 influenza A HA proteins are displayed on one 3-fold axis of a T33 particle whereas all H3N2 influenza A HA proteins are displayed on the other 3-fold axis of the T33 particle. This may be achieved by use a nanostructure comprising two or more pluralities of polypeptides as the core of the nanostructure with each plurality of polypeptides attached to a different antigen. Alternatively, a nanostructure may be engineered with one or more symmetry-breaking mutations, such as knob-in-hole mutations or intramolecular disulfide mutations, which have the effect of preventing trimer formation between the different antigens. In that case, the nanostructure displays multimeric antigens from different strains at symmetrically equivalent positions on the nanostructure, but each position on the nanostructure is occupied by homomers from the same strain, with only an insignificant proportion of inter-strain heteromeric antigens. In some cases, the antigen itself may be genetically engineered to prevent inter-strain heterodimerization. In an embodiment, the nanostructure is engineered to prevent heteromization of two antigenic proteins with conserved structure but divergent antigenicity, such as for example, an HA protein from the 2009 H1N1 California influenza and the HA protein from the 1999 H1N1 New Caledonia influenza. Furthermore, when mixed nanostructures are made and the antigens are displayed as fusion proteins, the nanostructure will comprise three or more different proteins, as the fusion proteins will share identical (or equivalent) domains used to form the core of the nanostructure with different antigenic domains, one for each antigen displayed on the nanostructure.

### 2.3. Attachment Modalities

The nanostructures of the present disclosure display antigens in various ways including as gene fusion or by other means disclosed herein. As used herein, “attached to” denotes any means known in the art for causing two polypeptides to associate. The association may be direct or indirect, reversible or irreversible, weak or strong, covalent or non-covalent, and selective or nonselective.

In some embodiments, attachment is achieved by genetic engineering to create an N- or C-terminus fusion of an antigen to one of the pluralities of polypeptides composing the nanostructure. Thus, the nanostructure may consist of, or consist essentially of, one, two, three, four, five, six, seven, eight, nine, or ten pluralities of polypeptides displaying one, two, three, four, five, six, seven, eight, nine, or ten pluralities of antigens, where at least one of the pluralities of antigen is genetically fused to at least one of the plurality of polypeptides. In some cases, the nanostructure consists essentially of one plurality of polypeptides capable of self-assembly and comprising the plurality of antigens genetically fused thereto. In some cases, the nanostructure consists essentially of a first plurality of polypeptides comprising the plurality of antigens genetically fused thereto; and a second plurality of polypeptides capable of co-assembling into two-component nanostructure, one plurality of polypeptides linking the antigen to the nanostructure and the other plurality of polypeptides promoting self-assembly of the nanostructure.

In some embodiments, attachment is achieved by post-translational covalent attachment between one or more pluralities of polypeptides and one or more pluralities of antigen. In some cases, chemical cross-linking is used to non-specifically attach the antigen to the nanostructure polypeptide. In some cases, chemical cross-linking is used to specifically attach the antigen to the nanostructure polypeptide. Various specific and non-specific cross-linking chemistries are known in the art, such as Click chemistry and other methods. In general, any cross-linking chemistry used to link two proteins may be adapted for use in the presently disclosed nanostructures. In particular, chemistries used in creation of immunoconjugates or antibody drug conjugates may be used. In some cases, an antigen-nanostructure conjugate (ANC) is created using a cleavable or non-cleavable linker. Processes and methods for conjugation of antigens to carriers are provided by, e.g., U.S. Patent Pub. No. US 2008/0145373 A1. In an embodiment, the antigen is a polysaccharide. In some cases, the antigen is a polysaccharide and the nanostructure acts as a hapten. In an embodiment, the target antigen is a protein and conjugation of the target antigen to a polysaccharide is used to enhance the immune response. Processes for preparing protein-polysaccharide conjugates are provided in, e.g., U.S. Pat. No. 6,248,334. The conjugation of proteins to polysaccharides in some cases converts a polysaccharide from a weakly immunogenic T-cell independent antigen to a T-cell dependent antigen that recruits T-cell help, and thus stimulates heightened immune responses. See J. M. Cruse, et al. (Editors), Conjugate Vaccines, Karger, Basel, (1989); and R. W. Ellis, et al. (Editors), Development and Clinical Uses of Haemophilus B Conjugate Vaccines, Marcel Dekker, New York (1994).

In an embodiment, attachment is achieved by non-covalent attachment between one or more pluralities of polypeptides and one or more pluralities of antigen. In some cases, the antigen is engineered to be negatively charged on at least one surface and the polypeptide is engineered to be positively charged on at least one surface, or positively and negatively charged, respectively. This promotes intermolecular association between the antigen and the polypeptides of the nanostructure by electrostatic force. In some cases, shape complementarity is employed to cause linkage of antigen to nanostructure. Shape complementarity can be pre-existing or rationally designed. In some cases, computational designed of protein-protein interfaces is used to achieve attachment. In an embodiment, the antigen is biotin-labeled and the polypeptide comprises a streptavidin, or vice versa. In an embodiment, streptavidin is displayed by gene fusion or otherwise as a tetramer on a 4-fold axis of the nanostructure and the biotin-labeled antigen is monomeric, dimeric, or tetrameric, permitting association to the nanostructure in a configuration appropriate for native multimerization of the antigen. In some cases, a protein-based adaptor is used to capture the antigen. In some cases, the polypeptide is fused to a protein capable of binding a complementary protein, which is fused to the antigen. In an embodiment, the polypeptide is fused to the rotavirus VP6 protein, which forms a trimer, and the antigen is N-terminally fused to the N-terminal peptide of rotavirus VP7, permitting trimer-to-trimer association of antigen to nanostructure. See Chen et al. Molecular interactions in rotavirus assembly and uncoating seen by high-resolution cryo-EM. PNAS 2009 June, 106 (26) 10644-10648.

In an embodiment, each of the first plurality of the antigenic proteins has a proximal end and a distal end, and the proximal ends of the antigenic proteins are each attached to a member of the first plurality of polypeptides. Thus, the distal end of the antigenic protein is defined as the portion of the antigen furthest from the center of the nanostructure. In an embodiment, the antigenic protein comprises target epitope, and the nanostructure is configured to display the target epitope. In some cases, the antigenic protein may comprise more than one target epitope and the nanostructure is configured to display each of the target epitopes. Epitopes progressively closer to the distal end are (without being bound by theory) in some cases preferentially accessible to the immune system. The distal end of the antigenic protein may be its N terminus, its C terminus, or neither terminus. Thus, depending on how the antigenic protein is attached to the nanostructure, the antigenic protein may be displayed in any orientation. In some cases, the antigenic protein is displayed so that one or more known epitopes are oriented at or towards the distal end of the antigenic protein, such that these epitope(s) are preferentially accessible to the immune system. In some cases, the orientation will recapitulate the orientation of a viral protein with respect to the virus. Thus, in the case of influenza HA, the antigenic protein HA may be oriented so that the receptor binding site is at the distal end of the protein, similar to the orientation of HA in the whole virus: or alternatively, the influenza HA protein may be oriented such that the stem epitope is preferentially accessible to the immune system. The choice of orientation may direct the immune system to one or the other epitope. In this example, the immune response to influenza may be guided to the receptor binding site or to the stem by choice of orientation. Similarly, the orientation of other antigens may influence the immune response. In some embodiments, orientation of the antigen results in an immune response targeted to a preferred epitope. In the case of HIV, the antigenic protein is in some embodiments the Env protein of HIV-1 or HIV-2, or an antigenic fragment thereof. The orientation of the Env or fragment thereof will in some cases recapitulate that the orientation of Env protein with respect to the HIV viron, such that the proximal end is the membrane-proximal end of the Env protein or fragment thereof. In some cases, the preferred epitope is selected from the group consisting of the CD4-binding site (CD4bs); the V2 proteoglycan moiety on the trimer apex of Env: the V3 proteoglycan moiety on the high mannose patch of Env: the membrane proximal external region (MPER) of the Env transmembrane domain: and the gp 120-gp41 interface with or without fusion peptide. In some cases, epitope preference is control by other means, such as positioning of glycans on the nanostructure by addition or subtraction of the N-linked glycan sequence motif N-X-[T/S] at predetermined positions in the amino acid sequence of any of the polypeptides of the nanostructure including in the amino acid sequence of the antigen. In some cases, the epitopes found at intermediate distances from the proximal to the distal end will be the preferred over epitopes more distally located depending on various considerations including but not limited to the overall geometry of the nanostructure, surface hydrophobicity, surface charge, and competitive binding of proteins endogenously present in the subject or proteins exogenously provided in the vaccine composition. The present disclosure encompasses all known methods of rational design of protein structure and the foregoing is not intended to be limiting.

### 2.4. Nanostructure Polypeptide Sequences

The one or more pluralities of polypeptides of the present disclosure may have any of various amino acids sequences. U.S. Patent Pub No. US 2015/0356240 A1 describes various methods for designing nanostructures. As disclosed in US Patent Pub No. US 2016/0122392 A1 and in International Patent Pub. No. WO 2014/124301 A1, the isolated polypeptides of SEQ ID NOS: 1-51 were designed for their ability to self-assemble in pairs to form nanostructures, such as icosahedral nanostructures. The design involved design of suitable interface residues for each member of the polypeptide pair that can be assembled to form the nanostructure. The nanostructures so formed include symmetrically repeated, non-natural, non-covalent polypeptide-polypeptide interfaces that orient a first assembly and a second assembly into a nanostructure, such as one with an icosahedral symmetry. Thus, in one embodiment the first and second polypeptides are selected from the group consisting of SEQ ID NOS:1-51. In each case, the N-terminal methionine residue is optional.

TABLE 1

| | | identified interface |
|---|---|---|
| Name | Amino Acid Sequence | residues |
| I53-34A | MEGMDFLAVLAESRLLPLLTVRGGEDLAGLATVLELMGVGALEITLRTEKGLE | I53-34A |
| SEQ ID NO: 1 | ALKALRKSGLLLGAGTVRSPKEAEAALEAGAAFLVSPGLLEEVAALAQARGVP | 28, 32, 36,37, 186, 188, 191, |
| | YLPGVLTPTEVERALALGLSALKFFPAEPFQGVRVLRAYAEVFPEVRFLPTGG | 192, 195 |
| | IKEEHLPHYAALPNLLAVGGSWLLQGDLAAVMKKVKAAKALLSPQAPG | |
| I53-34B | MTKKVGIVDTTFARVDMAEAAIRTLKALSPNIKIIRKTVTGIKDLPVACKKLL | I53-34B: |
| SEQ ID NO: 2 | EEEGCDIVMALGMPGKAEKDKVCAHEASLGLMLAQLMTNKHIIEVFVHEDEAK | 19, 20, 23, 24, 27, 109, 113, |
| | DDDELDILALVRAIEHAANVYYLLFKPEYLTRMAGKGLRQGREDAGPARE | 116, 117, 120, 124, 148 |
| I53-40A | MTKKVGIVDTTFARVDMASAAILTLKMESPNIKIIRKTVPGIKDLPVACKKLL | 153-40A: |
| SEQ ID NO: 3 | EEEGCDIVMALGMPGKAEKDKVCAHEASLGLMLAQLMTNKHIIEVFVHEDEAK | 20, 23, 24, 27, 8,109, 12, |
| | DDAELKILAARRAIEHALNVYYELLFKPEYLTRMAGKGLRQGFEDAGPARE | 113, 116, 120, 124 |
| 153-40B | MSTINNQLKALKVIPVIAIDNAEDIIPLGKVLAENGLPAAEITFRSSAAVKAI | 153-40B: |
| SEQ ID NO: 4 | MLLRSAQPEMLIGAGTILNGVQALAAKEAGATFVVSPGFNPNTVRACQIIGID | 47, 51, 54, 58, 74, 102 |
| | IVPGVNNPSTVEAALEMGLTTLKFFPAEASGGISMVKSLVGPYGDIRLMPTGG | |
| | ITPSNIDNYLAIPQVLACGGTWMVDKKLVTNGEWDEIARLTREIVEQVNP | |
| I53-47A | MPIFTLNTNIKATDVPSDFLSLTSRLVGLILSKPGSYVAVHINTDQQLSFGGS | I53-47A: |
| SEQ ID NO: 5 | TNPAAFGTLMSIGGIEPSKNRDHSAVLEDHLNAMLGIPKNRMYIHFVNLNGDD | 22, 25, 29, 72, 79, 86, 87 |
| | VGWNGTTF | |
| I53-47B | MNQHSHKDYETVRIAVVRARWHADIVDACVEAFEIAMAAIGGDRFAVDVFDVP | I53-47B: |
| SEQ ID NO: 6 | GAYEIPLHARTLAETGRYGAVLGTAFVVNGGIYRHEFVASAVIDGMMNVQLST | 28, 31, 35, 36, 39, 131, 132, |
| | GVPVLSAVLTPHRYRDSAEHHRFFAAHFAVKGVEAARACIEILAAREKIAA | 135, 139, 146 |
| I53-50A | MKMEELFKKHKIVAVLRANSVEEAIEKAVAVFAGGVHLIEITFTVPDADTVIK | I53-50A: 25, 29, 33, 54, 57 |
| SEQ ID NO: 7 | ALSVLKEKGAIIGAGTVTSVEQCRKAVESGAEFIVSPHLDEEISQFCKEKGVF | |
| | YMPGVMTPTELVKAMKLGHTILKLFPGEVVGPQFVKAMKGPFPNVKFVPTGGV | |
| | NLDNVCEWFKAGVLAVGVGSALVKGTPDEVREKAKAFVEKIRGCTE | |
| 153-50B | MNQHSHKDYETVRIAVVRARWHAEIVDACVSAFEAAMADIGGDRFAVDVFDVP | I53-50B: |
| SEQ ID NO: 8 | GAYEIPLHARTLAETGRYGAVLGTAFVVNGGIYRHEFVASAVIDGMMNVQLST | 24, 28, 36, 124, 125, 127, 128, |
| | GVPVLSAVLTPHRYRDSDAHTLLFLALFAVKGMEAARACVEILAAREKIAA | 129, 131, 132, 133, 135, 139 |
| I53-51A | MFTKSGDDGNTNVINKPVGKDSPLVNFLGDLDELNSFIGFAISKIPWEDMKKD | I53-51A: |
| SEQ ID NO: 9 | LERVQVELFEIGEDLSTQSSKKKIDESYVLWLLAATAIYRIESGPVKLFVIPG | 80, 83, 86, 87, 88, 90, 91, 94, |
| | GSEEASVLHVTRSVARRVERNAVKYTKELPEINREIIVYLNRLSSLLFAMALV | 166, 172, 176 |
| | ANKRRNQSEKIYEIGKSW | |
| I53-51B | MNQHSHKDYETVRIAVVRARWHADIVDQCVRAFEEAMADAGGDRFAVDVFDVP | I53-51B: |
| SEQ ID NO: 10 | GAYEIPLHARTLAETGRYGAVLGTAFVVNGGIYRHEFVASAVIDGMMNVQLST | 31, 35, 36, 40, 122, 124, 128, |
| | GVPVLSAVLTPHRYRSSREHHEFFREHFMVKGVEAAAACITILAAREKIAA | 131, 135, 139, 143, 146, 147 |
| I52-03A | MGHTKGPTPQQHDGSALRIGIVHARWNKTIIMPLLIGTIAKLLECGVKASNIV | I52-03A: |
| SEQ ID NO: 11 | VQSVPGSWELPIAVQRLYSASQLQTPSSGPSLSAGDLLGDSTTDLTALPTTTA | 28, 32, 36, 39, 44, 49 |
| | SSTGPFDALIAIGVLIKGETMHFEYIADSVSHGLMRVQLDTGVPVIFGVLTVL | |
| | TDDQAKARAGVIEGSHNHGEDWGLAAVEMGVRRRDWAAGKTE | |
| I52-C3B | MYEVDHADVYDLFYLGRGKDYAAEASDIADLVRSRTPEASSLLDVACGTGTHL | I52-03B: |
| SEQ ID NO: 12 | EHFTKEFGDTAGLELSEDMLTHARKRLPDATLHQGDMRDFQLGRKFSAVVSMF | 94, 115, 116, 206, 213 |
| | SSVGYLKTVAELGAAVASFAEHLEPGGVVVVEPWWFPETFADGWVSADVVRRD | |
| | GRTVARVSHSVREGNATRMEVHFTVADPGKGVRHFSDVHLITLFHQREYEAAF | |
| | MAAGLRVEYLEGGPSGRGLFVGVPA | |
| I52-32A | MGMKEKFVLIITHGDFGKGLLSGAEVIIGKQENVHTVGLNLGDNIEKVAKEVM | I52-32A: |
| SEQ ID NO: 13 | RIIIAKLAEDKEIIIVVDLFGGSPFNIALEMMKTFDVKVITGINMPMLVELLT | 47, 49, 53, 54, 57, 58, 61, 83, |
| | SINVYDTTELLENISKIGKDGIKVIEKSSLKM | 87, 88 |
| I52-32B | MKYDGSKLRIGILHARWNLEIIAALVAGAIKRLQEFGVKAENIIIETVPGSFE | I52-32B: 19, 20, 23, 30, 40 |
| SEQ ID NO: 14 | LPYGSKLFVEKQKRLGKPLDAIIPIGVLIKGSTMHFEYICDSTTHQLMKLNFE | |
| | LGIPVIFGVLTCLTDEQAEARAGLIEGKMHNHGEDWGAAAVEMATKFN | |
| 152-33A | MAVKGLGEVDQKYDGSKLRIGILHARWNRKIILALVAGAVLRLLEFGVKAENI | I52-33A: 33, 41, 44, 50 |
| SEQ ID NO: 15 | IIETVPGSFELPYGSKLFVEKQKRLGKPLDAIIPIGVLIKGSMHFTEYICDST | |
| | THQLMKLNFELGIPVIFGVLTCLTDEQAEARAGLIEGKMHNHGEDWGAAAVEM | |
| | ATKFN | |
| I52-33B | MGANWYLDNESSRLSFTSTKNADIAEVHRFLVLHGKVDPKGLAEVEVETESIS | I52-33B: |
| SEQ ID NO: 16 | TGIPLPDMLLRVLVFQVSKFPVAQINAQLDMRPINNLAPGAQLELRLPLTVSL | 61, 63, 66, 67, 72, 147, 148, |
| | RGLSHSYNAELLATRLDERRFQVVTLEPLVIHAQDFDMVRAFNALRLVAGLSA | 154, 155 |
| | VSLSVPVGAVLIFTAR | |
| I32-06A | MTDYIRDGSAIKALSFAIILAEADLRHIPQDLQRLAVRVIHACGMVDVANDLA | I32-06A: |
| SEQ ID NO: 17 | FSEGAGKAGRNALLAGAPILCDARMVAEGITRSRLPADNRVIYTLSDPSVPEL | 9, 12, 13, 14, 20, 30, 33, 34 |
| | AKKIGNTRSAAALDLWLPHIEGSIVAIGNAPTALFRLFELLDAGAPKPALIIG | |
| | MPVGFVGAAESKDELAANSRGVPYVIVRGRRGGSAMTAAAVNALASERE | |
| I32-06B | MITVFGLKSKLAPRREKLAEVIYSSLHLGLDIPKGKHAIRFLCLEKEDFYYPF | I32-06B: |
| SEQ ID NO: 18 | DRSDDYTVIEINLMACRSEETKMLLIFLLFIALERKLGIRAHDVEITIKEQPA | 24, 71, 73, 76, 77, 80, 81, 84, |
| | HCWGFRGRTGDSARDLDYDIYV | 85, 83, 114, 118 |
| I32-19A | MGSDLQKLQRFSTCDISDGLLNVYNIPTGGYFPNLTAISPPQNSSIVGTAYTV | I32-19A: |
| SEQ ID NO: 19 | LFAPIDDPRPAVNYIDSVPPNSILVLALEPHLQSQFHPFIKITQAMYGGLMST | 208, 213, 218, 222, 225, 226, |
| | RAQYLKSNGTVVFGRIRDVDEHRTLNHPVFAYGVGSCAPKAVVKAVGTNVQLK | 229, 233 |
| | ILTSDGVTQTICPGDYIAGDNNGIVRIPVQETDISKLVTYIEKSIEVDRLVSE | |
| | AIKNGLPAKAAQTARRMVLKDY | |
| I32-19B | MSGMRVYLGADHAGYELKQAIIAFLKMTGHEPIDCGALRYDADDDYPAFCIAA | I32-19B |
| SEQ ID NO: 20 | ATRTVADPGSLGIVLGGSGNGEQIAANKVPGARCALAWSVQTAALAREHNNAQ | 20, 23, 24, 27, 117, 118, 122, |
| | LIGIGGRMHTLEEALRIVKAFVTTPWSKAQRHQRRIDILAEYERTHEAPPVPG | 125 |
| | APA | |
| I32-28A | MGDDARIAAIGDVDELNSQIGVLLAEPLPDDVRAALSAIQHDLFDLGGELCIP | I32-28A: |
| SEQ ID NO: 21 | GHAAITEDHLLRLALWLVHYNGQLPPLEEFILPGGARGAALAHVCRTVCRRAE | 60, 61, 64, 67, 68, 71, 110, |
| | RSIKALGASEPLNIAPAAYVNLLSDLLFVLARVLNRAAGGADVLWDRTRAH | 120, 123, 124, 128 |
| I32-28B | MILSAEQSFTLRRPHGQAAALAFVREPAAALAGVQRLRGLDSDGEQVWGELLV | I32-28B: |
| SEQ ID NO: 22 | RVPLLGEVDLPFRSEIVRTPQGAELRPLTLTGERAWVAVSGQATAAEGGEMAF | 35, 36, 54, 122, 129, 137, 140, |
| | AFQFQAHLATPEAEGEGGAAFEVMVQAAAGVTLLLVAMALPQGLAAGLPPA | 141, 144, 148 |
| I53-40A.1 | MTKKVGIVDTTFARVDMASAAILTLKMESPNIKIIRKTVPGIKDLPVACKKLL | I53-40A: |
| SEQ ID NO: 23 | EEEGCDIVMALGMPGKKEKDKVCAHEASLGLMLAQLMTNKHIIEVFVHEDEAK | 20, 23, 24, 27, 28, 109, 112, |
| | DDAELKILAARRAIEHALNVYYLLFKPEYLTRMAGKGLRQGFEDAGPARE | 113, 116, 120, 124 |
| I53-40B.1 | MDDINNQLKRLKVIPVIAIDNAEDIIPLGKVLAENGLPAAEITFRSSAAVKAI | I53-40B: |
| SEQ ID NO: 24 | MLLRSAQPEMLIGAGTILNGVQALAAKEAGADFVVSPGFNPNTVRACQIIGID | 47, 51, 54, 58, 4, 102 |
| | IVPGVNNPSTVEQALEMGLTTLKFFPAEASGGISMVKSLVGPYGDIRLMPTGG | |
| | ITPDNIDNYLAIPQVLACGGTWMVDKKLVRNGEWDEIARLTREIVEQVNP | |
| I53-47A.1 | MPIFTLNTNIKADDVPSDFLSLTSRLVGLILSKPGSYVAVHINTDQQLSFGGS | I53-47A: |
| SEQ ID NO: 25 | TNPAAFGTLMSIGGIEPDKNRDHSAVLFDHLNAMLGIPKNRMYIHFVNLNGDD | 22, 25, 29, 72, 79, 86, 87 |
| | VGWNGTTF | |
| I53- | MPIFTLNTNIKADDVPSDFLSLTSRLVGLILSEPGSYVAVHINTDQQLSFGGS | I53-47A: |
| 47A.1NegT2 | TNPAAFGTLMSIGGIEPDKNEDHSAVLFDHLNAMLGIPENRKYIHFVDLDGDD | 22, 25, 29, 72, 79, 86, 87 |
| SEQ ID NO: 26 | VGWNGTTF | |
| I53-47B.1 | MNQHSHKDHETVRIAVVRARWHADIVDACVEAFEIAMAAIGGDRFAVDVFDVP | 153-47B: |
| SEQ ID NO: 27 | GAYEIPLHARTLAETGRYGAVLGTAFVVNGGIYRHEFVASAVIDGMMNVQLDT | 28, 31, 35, 36, 39, 131, 132, |
| | GVPVLSAVLTPHRYRDSDEHHRFFAAHFAVKGVEAARACIEILNAREKIAA | 135, 139, 146 |
| I53- | MNQHSHKDHETVRIAVVPARWHADIVDACVEAFEIAMAAIGGDRFAVDVFDVP | I53-47B: |
| 47B.1NegT2 | GAYEIPLHARTLAETGRYGAVLGTAFVVDGGIYDHEFVASAVIDGMMNVQLDT | 28, 31, 35, 36, 39, 131, 132, |
| SEQ ID NO: 28 | GVPVLSAVLTPHEYEDSDEDHEFFAAHFAVKGVEAARACIEILNAREKIAA | 135, 139, 146 |
| I53-50A.1 | MKMEELFKKHKIVAVLRANSVEEAIEKAVAVFAGGVHLIEITFTVPDADTVIK | 153-50A: 25, 29, 33, 54, 57 |
| SEQ ID NO: 29 | ALSVLKEKGAIIGAGTVTSVEQCRKAVESGAEFIVSPHLDEEISQFCKEKGVF | |
| | YMPGVMTPTELVKAMKLGHDILKLFPGEVVGPQFVKAMKGPFPNVKFVPTGGV | |
| | NLDNVCEWFKAGVLAVGVGDALNKGDPDEVREKAKKFEVEKIRGCTE | |
| 153- | MKMEELFKKHKIVAVLRANSVEEAIEKAVAVFAGGVHLIEITFTVPDADTVIK | 153-50A: 25, 29, 33, 54, 57 |
| 50A.1NegT2 | ALSVLKEKGAIIGAGTVTSVEQCRKAVESGAEFIVSPHLDEEISQFCKEKGVF | |
| SEQ ID NO: 30 | YMPGVMTPTELVKAMKLGHDILKLFPGEVVGPEFVEAMKGPFPNVKFVPTGGV | |
| | DLDDVCEWFDAGVLAVGVGDALVEGDPDEVREDAKEFVEEIRGCTE | |
| I53- | MKMEELFKKHKIVAVLRANSVEEAIEKAVAVFAGGVHLIEITFTVPDADTVIK | I53-50A: 25, 29, 33, 54, 57 |
| 50A.1PosT1 | ALSVLKEKGAIIGAGTVTSVEQCRKAVESGAEFIVSPHLDEEISQFCKEKGVF | |
| SEQ ID NO: 31 | YMPGVMTPTELVKAMKLGHDILKLFPGEVVGPQFVKAMKGPFPNVKFVPTGGV | |
| | NLDNVCKWFKAGVLAVGVGKALVKGKPDEVREKAKKFVKKIRGCTE | |
| I53 50B.1 | MNQHSHNDHETVRIAVVRAPWHAEIVDACVSAFEAAMRDIGGDRFAVDVFDVP | I53-50B |
| SEQ ID NO: 32 | GAYEIPLHARTLAETGRYGAVLGTAFVVNGGIYRHEFVASAVIDGMMNVQLDT | 24, 28, 36, 124, 125, 127, 128, |
| | GVPVLSAVLTPHRYRDSDAHTLLFLALFAVKGMEAARACVEILAAREKIAA | 129, 131, 132, 133, 135, 139 |
| I53- | MNQHSHKDHETVRIAVVRARWHAEIVDACVSAFEAAMRDIGGDRFAVDVFDVP | I53-50B: |
| 50B.1NegT2 | GAYEIPLHARTLAETGRYGAVLGTAFVVDGGIYDHEFVASAVIDGMMNVQLDT | 24, 28, 36, 124, 125, 127, 128, |
| SEQ ID NO: 33 | GVPVLSAVLTPHEYEDSDADTLLFLALFAVKGMEAARACVEILAAREKIAA | 129, 131, 132, 133, 135, 139 |
| I53- | MNQHSHKDHETVRIAVVRARWHAEIVDACVSAFEAAMRDIGGDRFAVDVFDVP | I53-50B: |
| 50B.4PosT1 | GAYEIPLHARTLAETGRYGAVLGTAFVVNGGIYRHEFVASAVINGMMNVQLNT | 24, 28, 36, 124, 125, 127, 128, |
| SEQ ID NO: 34 | GVPVLSAVLTPHNYDKSKAHTLLFLALFAVKGMEAARACVEILAAREKIAA | 129, 131, 132, 133, 135, 139 |
| I53-40 A | MTKKVGIVDTTFARVDMASAAILTLKMESPNIKIIRKTVPGIKDLPVACKKLL | |
| genus | EEEGGDIVMALGMPGK(A/K)EKDKVCAHEASLGLMLAQLMTNKHIIEVFVHE | |
| SEQ ID NO: 35 | DEAKDDAELKILAARRAIEHALNVYYLLFKPEYLTRMAGKGLRQGFEDAGPAR | |
| | E | |
| I53-40 B | M(S/D)(T/D)INNQLK(A/R)LKVIPVIAIDNAEDIIPLGKVLAENGLPAAE | |
| genus | ITFRSSAAVKAIMLLRSAQPEMLIGAGTILNGVQALAAKEAGA(T/D)FVVSP | |
| SEQ ID NO: 36 | GFNPNTVRACQIIGIDIVPGVNNPSTVE(A/Q)ALEMGLTTLKFFPAEASGGI | |
| | SMVKSLVGPYGDIRLMPTGGITP(S/D)NIDNYLAIPQVLACGGTWMVDKKLV | |
| | (T/R)NGEWDEIARLTREIVEQVNP | |
| I53-47A genus | MPIFTLNTNIKA(T/D)DVPSDFLSLTSRLVGLILS(K/E)PGSYVAVHINTD | |
| SEQ ID NO: 37 | QQLSFGGSTNPAAFGTLMSIGGIEP(S/D)KN(R/E)DHSAVLFDHLNAMLGI | |
| | PKNRMYIHFV(N/D)L(N/D)GDDVGWNGTTF | |
| I53-47B genus | MNQHSHKD(Y/H)ETVRIAVVRARWHADIVDACVEAFEIAMAAIGGDRFAVDV | |
| SEQ ID NO: 38 | FDVPGAYEIPLHARTLAETGRYGAVLGTAFVV(N/D)GGIY(R/D)HEFVASA | |
| | VIDGMMNVQL(S/D)TGVPVLSAVLTPH(R/E)Y(R/E)DS(A/D)E(H/D)H | |
| | (R/E)FFAAHFAVKGVEAARACIEIL(A/N)AREKIAA | |
| 153-50A genus | MKMEELFKKHKIVAVLRANSVEEAIEKAVAVFAGGVHLIEITFTVPDADTVIK | |
| SEQ ID NO: 39 | ALSVLKEKGAIIGAGTVTSVEQCRKAVESGAEFIVSPHLDEEISQFCKEKGVF | |
| | YMPGVMTPTELVKAMKLGH(T/D)ILKLFPGEVVGP(Q/E)FV(K/E)AMKGP | |
| | FPNVKFVPTGGV(N/D)LD(N/D)VC(E/K)WF(K/D)AGVLAVGVG(S/K/D) | |
| | ALV(K/E)G(T/D/K)PDEVRE(K/D)AK(A/E/K)FV(E/K)(K/E)IRGCT | |
| | E | |
| I53-50B genus | MNQHSHKD(Y/H)ETVRIAVVRARWHAEIVDACVSAFEAAM(A/R)DIGGDRF | |
| SEQ ID NO: 40 | AVDVFDVPGAYEIPLHARTLAETGRYGAVLGTAFVV(N/D)GGIY(R/D)HEF | |
| | VASAVI(D/N)GMMNVQL(S/D/N)TGVPVLSAVLTPH(R/E/N)Y(R/D/E) | |
| | (D/K)S(D/K)A(H/D)TLLFLALFAVKGMEAARACVEILAAREKIAA | |
| T32-28A | MGEVPIGDPKELNGMEIAAVYLQPIEMEPRGIDLAASLADIHLEADIHALKNN | |
| SEQ ID NO: 41 | PNGFPEGFWMPYLTIAYALANADTGAIKTGTLMPMVADDGPHYGANIAMEKDK | |
| | KGGFGVGTYALTFLISNPEKQGFGRHVDEETGVGKWFEPFVVTYFFKYTGTPK | |
| T32-28B | MSQAIGILELTSIAKGMELGDAMLKSANVDLLVSKTISPGKFLLMLGGDIGAI | |
| SEQ ID NO: 42 | QQAIETGTSQAGEMLVDSLVLANIHPSVLPAISGLNSVDKRQAVGIVETWSVA | |
| | ACISAADLAVKGSNVTLVRVHMAFGIGGKCYMVVAGDVLDVAAAVATASLAAG | |
| | AKGLLVYASIIPRPHEAMWRQMVEG | |
| T33-09A | MEEVVLITVPSALVAVKIAHALVEERLAACVNIVPGLTSIYRWQGSVVSDHEL | |
| SEQ ID NO: 43 | LLLVKTTTHAFPKLKERVKALHPYTVPEIVALPIAEGNREYLDWLRENTG | |
| T33-09B | MVRGIRGAITVEEDTPAAILAATIELLLKMLEANGIQSYEELAAVIFTVTEDL | |
| SEQ ID NO: 44 | TSAFPAEAARLIGMHRVPLLSAREVPVPGSLPRVIRVLALWNTDTPQDRVRHV | |
| | YLNEAVRLRPDLESAQ | |
| T33-15A | MSKAKIGIVTVSDRASAGITADISGLAIILALNLYLTSEWEPIYQVIPDEQDV | |
| SEQ ID NO: 45 | IETTLIKMADEQDCCLIVTTGGTGPAKRDVTPEATEAVCDRMMPGFGELMRAE | |
| | SLKEVPTAILSRQTAGLRGDSLIVNLPGDPASISDCLLAVFPAIPYCIDLMEG | |
| | PYLECNEAMIKPFRPKAK | |
| T33-15B | MVRGIRGAITVNSDTPTSIIIATILLLEKMLEANGIQSYEELAAVIFTVTEDL | |
| SEQ ID NO: 46 | TSAFPAEAARQIGMHRVPLLSAREVPVPGSLPPVIRVLALWNTDTPQDRVRHV | |
| | YLSEAVRLRPDLESAQ | |
| T33-21A | MRITTKVGDKGSTRLFGGEEVWKDSPIIEANGTLDELTSFIGEAKHYVDEEMK | |
| SEQ ID NO: 47 | GILEEIQNDIYKIMGEIGSKGKIEGISEERIAWLLKLILRYMEMVNLKSFVLP | |
| | GGTLESAKLDVCRTIARRALRKVLTVTREFGIGAEAAAYLLALSDLLFLLARV | |
| | IEIEKNLLKEVRS | |
| T33-21B | MPHLVIEATANLRLETSPGELLEQANKALFASGQFGEADIKSRFVTLEAYRQG | |
| SEQ ID NO: 48 | TAAVERAYLHACLSILDGRDIATPTLLGASLCAVLAEAVAGGGEEGVQVSVEV | |
| | REMERLSYAKRVVARQR | |
| T33-28A | MESVNTSFLSPSLVTIRDFDNGQFAVLRIGRTGFPADKGDIDLCLDKMIGVRA | |
| SEQ ID NO: 49 | AQIFLGDDTEDGFKGPHIRIRCVDIDDKHTYNAMVYVDLIVGTGASEVERETA | |
| | EEEAKLALRVALQVDIADEHSCVTQFEMKLREELLSSDSFHPDKDEYYKDFL | |
| T33-28B | MPVIQTFVSTPLDHHKRLLLAIIYRIVTRVVLGKPEDLVMMTFHDSTPMHFFG | |
| SEQ ID NO: 50 | STDPVACVRVEALGGYGPSEPEKVTSIVTAAITAVCGIVADRIFVLYFSPLHC | |
| | GWNCTNE | |
| T33-31A | MEEVVLITVPSALVAVKIAHALVEERLAACVNIVPGLTSIYREEGSVVSDHEL | |
| SEQ ID NO: 51 | LLLVKTTTDAFPKLKERVKELHPYEPEIVALPIAEGNREYLDWLRENTG | |

Table 1 provides the amino acid sequence of the first and second polypeptides from embodiments of the present disclosure. In each case, the pairs of sequences together from an I53 icosahedron. The right hand column in Table 1 identifies the residue numbers in each exemplary polypeptide that were identified as present at the interface of resulting assembled nanostructures (i.e.: “identified interface residues”). As can be seen, the number of interface residues for the exemplary polypeptides of SEQ ID NO: 1-34 range from 4-13. In various embodiments, the first and second polypeptides comprise an amino acid sequence that is at least 75%, 80%, 85%, 90%, 91%, 92%, 93%, 94%, 95%, 96%, 97%, 98%, or 99% identical over its length, and identical at least at 1, 2, 3, 4, 5, 6, 7, 8, 9, 10, 11, 12, or 13 identified interface positions (depending on the number of interface residues for a given polypeptide), to the amino acid sequence of a polypeptide selected from the group consisting of SEQ ID NOS: 1-34. SEQ ID NOs: 35-51 represent other amino acid sequences of the first and second polypeptides from embodiments of the present disclosure. In other embodiments, the first and second polypeptides comprise an amino acid sequence that is at least 75%, 80%, 85%, 90%, 91%, 92%, 93%, 94%, 95%, 96%, 97%, 98%, or 99% identical over its length, and identical at least at 20%, 25%, 33%, 40%, 50%. 60%, 70%, 75%, 80%, 90%, or 100% of the identified interface positions, to the amino acid sequence of a polypeptide selected from the group consisting of SEQ ID NOS: 1-51.

As is the case with proteins in general, the polypeptides are expected to tolerate some variation in the designed sequences without disrupting subsequent assembly into nanostructures: particularly when such variation comprises conservative amino acid substitutions. As used here, “conservative amino acid substitution” means that: hydrophobic amino acids (Ala, Cys, Gly, Pro, Met, Val, Ile, Leu) can only be substituted with other hydrophobic amino acids: hydrophobic amino acids with bulky side chains (Phe, Tyr, Trp) can only be substituted with other hydrophobic amino acids with bulky side chains; amino acids with positively charged side chains (Arg, His, Lys) can only be substituted with other amino acids with positively charged side chains: amino acids with negatively charged side chains (Asp, Glu) can only be substituted with other amino acids with negatively charged side chains; and amino acids with polar uncharged side chains (Ser, Thr, Asn, Gln) can only be substituted with other amino acids with polar uncharged side chains.

In various embodiments of the nanostructure of the invention, the first polypeptides and the second polypeptides comprise polypeptides with the amino acid sequence selected from the following pairs, or modified versions thereof (i.e.: permissible modifications as disclosed for the polypeptides of the invention: isolated polypeptides comprising an amino acid sequence that is at least 75% identical over its length, and/or identical at least at one identified interface position, to the amino acid sequence indicated by the SEQ ID NO.):
SEQ ID NO: 1 and SEQ ID NO:2 (I53-34A and I53-34B);
SEQ ID NO:3 and SEQ ID NO:4 (I53-40A and I53-40B);
SEQ ID NO:3 and SEQ ID NO:24 (I53-40A and I53-40B.1);
SEQ ID NO:23 and SEQ ID NO:4 (I53-40A.1 and I53-40B);
SEQ ID NO:35 and SEQ ID NO:36 (I53-40A genus and I53-40B genus);
SEQ ID NO:5 and SEQ ID NO:6 (I53-47A and I53-47B);
SEQ ID NO:5 and SEQ ID NO:27 (I53-47A and I53-47B.1);
SEQ ID NO:5 and SEQ ID NO:28 (I53-47A and I53-47B.1NegT2);
SEQ ID NO:25 and SEQ ID NO:6 (I53-47A.1 and I53-47B);
SEQ ID NO:25 and SEQ ID NO:27 (I53-47A.1 and I53-47B.1);
SEQ ID NO:25 and SEQ ID NO:28 (I53-47A.1 and I53-47B.1NegT2);
SEQ ID NO:26 and SEQ ID NO:6 (I53-47A.1NegT2 and I53-47B);
SEQ ID NO:26 and SEQ ID NO:27 (I53-47A.1NegT2 and I53-47B.1);
SEQ ID NO:26 and SEQ ID NO:28 (I53-47A.1NegT2 and I53-47B.1NegT2);
SEQ ID NO:37 and SEQ ID NO:38 (I53-47A genus and I53-47B genus):
SEQ ID NO: 7 and SEQ ID NO:8 (I53-50A and I53-50B);
SEQ ID NO:7 and SEQ ID NO:32 (I53-50A and I53-50B.1);
SEQ ID NO:7 and SEQ ID NO:33 (I53-50A and I53-50B.1NegT2);
SEQ ID NO:7 and SEQ ID NO:34 (I53-50A and I53-50B.4PosT1):
SEQ ID NO:29 and SEQ ID NO:8 (I53-50A.1 and I53-50B);
SEQ ID NO:29 and SEQ ID NO:32 (I53-50A.1 and I53-50B.1);
SEQ ID NO:29 and SEQ ID NO:33 (I53-50A.1 and I53-50B.1NegT2):
SEQ ID NO:29 and SEQ ID NO:34 (I53-50A.1 and I53-50B.4PosT1);
SEQ ID NO:30 and SEQ ID NO:8 (I53-50A.1NegT2 and I53-50B);
SEQ ID NO:30 and SEQ ID NO: 32 (I53-50A.1NegT2 and I53-50B.1);
SEQ ID NO:30 and SEQ ID NO:33 (I53-50A.1NegT2 and I53-50B.1NegT2);
SEQ ID NO:30 and SEQ ID NO:34 (I53-50A.1NegT2 and I53-50B.4PosT1);
SEQ ID NO:31 and SEQ ID NO:8 (I53-50A.1PosT1 and I53-50B);
SEQ ID NO:31 and SEQ ID NO:32 (I53-50A.1PosT1 and I53-50B.1);
SEQ ID NO:31 and SEQ ID NO:33 (I53-50A.1PosT1 and I53-50B.1NegT2);
SEQ ID NO:31 and SEQ ID NO:34 (I53-50A.1PosT1 and I53-50B.4PosT1):
SEQ ID NO:39 and SEQ ID NO:40 (I53-50A genus and I53-50B genus);
SEQ ID NO:9 and SEQ ID NO: 10 (I53-51A and I53-51B);
SEQ ID NO:11 and SEQ ID NO:12 (I52-03A and I52-03B);
SEQ ID NO:13 and SEQ ID NO: 14 (I52-32A and I52-32B);
SEQ ID NO:15 and SEQ ID NO: 16 (I52-33A and I52-33B);
SEQ ID NO: 17 and SEQ ID NO: 18 (I32-06A and I32-06B);
SEQ ID NO: 19 and SEQ ID NO:20 (I32-19A and I32-19B);
SEQ ID NO:21 and SEQ ID NO:22 (I32-28A and I32-28B);
SEQ ID NO:23 and SEQ ID NO:24 (I53-40A.1 and I53-40B.1);
SEQ ID NO:41 and SEQ ID NO:42 (T32-28A and T32-28B);
SEQ ID NO:43 and SEQ ID NO:44 (T33-09A and T33-09B);
SEQ ID NO:45 and SEQ ID NO:46 (T33-15A and T33-15B);
SEQ ID NO:47 and SEQ ID NO:48 (T33-21A and T33-21B);
SEQ ID NO:49 and SEQ ID NO:50 (T33-28A and T32-28B); and
SEQ ID NO:51 and SEQ ID NO:44 (T33-31A and T33-09B (also referred to as T33-31B))

In one embodiment, the one or more proteins, or antigenic fragments thereof, are expressed as a fusion protein with the first and/or second polypeptides. In these embodiments, one or more proteins, or antigenic fragments thereof are present at the N terminus of the fusion protein, whenever this configuration can facilitate presentation of the one or more proteins, or antigenic fragments thereof on an exterior of the nanostructure. A preference for the presence of the protein at the N terminus of the fusion protein occurs whenever from the location of the C terminus of the proteins is at proximal end of the protein. In these embodiments, one or more proteins, or antigenic fragments thereof are present at the C terminus of the fusion protein, whenever this configuration can facilitate presentation of the one or more proteins, or antigenic fragments thereof on an exterior of the nanostructure. A preference for the presence of the protein at the C terminus of the fusion protein occurs whenever from the location of the M terminus of the proteins is at proximal end of the protein.

Non-limiting examples of nanostructures useful in vaccines of the present disclosure include those disclosed in U.S. Pat. No. 9,630,994 and U.S. Provisional Patent Application No. 62/481,331, which are incorporated herein in its entirety.

### 3. Antigens

The present disclosure provides nanostructure-based vaccines for any of the various known bacteria, viruses, or parasites relevant to human or animal disease. In particular, the present disclosure relates to vaccines for lyme disease, pertussis, herpes virus, orthomyxovirus, paramyxovirus, pneumovirus, filovirus, flavivirus, reovirus, retrovirus. malaria, viral meningitis, fungal meningitis, and bacterial meningitis including Neisseria meningitides (also known as “meningococcus”), Haemophilus influenzae type B, Streptococcus pneumonia, and Listeria monocytogenes. For each of these organism, antigens (proteins or polysaccharides) capable of generating protective immune responses are known. The present disclosure relates to incorporation of any of these antigens—particularly antigenic proteins—into nanostructure-based vaccines. Guidance is particularly available from studies of the immune response to infection or vaccination, such as isolation of binding or neutralizing antibodies, genetic analysis of antigen sequence, structural studies of antigenic proteins and antibodies, and most particularly clinical and veterinary experience with subunit vaccines. With few limitations, any known subunit vaccine can be adapted for use with the nanostructures of the present disclosure by employing the display modalities provided above. In some embodiments, the nanostructure-based vaccines of the present disclosure comprise an oligosaccharide (e.g., a meningococcal oligosaccharide) conjugated directly or through an intermediate protein (e.g., diphtheria toxoid, tetanus toxoid, or CRM197) to the nanostructure. In some embodiments, the nanostructure-based vaccines of the present disclosure comprise antigens or antigenic fragments from the list provided in Table 2.

TABLE 2
Non-Limiting List of Antigens

| Infectious | | |
|---|---|---|
| Agent | Antigens | Citation |
| HIV | gp160, gp140, | Sok, D., Le, K. M., Vadnais, M., Saye-Francisco, K. |
| | gp21, MPER | L., Jardine, J. G., Torres, J. L., et al. (2017). Rapid |
| | | elicitation of broadly neutralizing antibodies to HIV |
| | | by immunization in cows. Nature, 548 (7665), 108-111. |
| RSV | F protein | US20160046675A1, US 2016/0031972 A1, |
| | (prefusion) | US 2017/0182151 A1, WO 2010/149745 A1, |
| | | WO 2012/158613 A1, WO 2013/139916 A1, |
| | | WO 2014/079842 A1, WO 2014/174018 A1, |
| | | WO 2014/202570 A1, WO 2015/013551 A1, |
| | | WO 2017/040387 A2, WO2017172890A1 |
| Influenza | HA-Influenza | Nabel et al. Induction of unnatural immunity: |
| | A and B | prospects for a broadly protective universal influenza |
| | | vaccine. Nat Med. 2010 December; 16 (12): 1389-91. |
| EBV | glycoprotein | Kanekiyo et al. Rational Design of an Epstein-Barr |
| | 350/220 (gp350) | Virus Vaccine Targeting the Receptor-Binding Site. |
| | | Cell. 2015 Aug. 27; 162 (5): 1090-100. |
| CMV | gB; UL128, | Ciferri et al. Structural and biochemical studies of |
| | UL130, UL131A, | HCMV gH/gL/gO and Pentamer reveal mutually |
| | gH (UL75) and gL | exclusive cell entry complexes. Proc. Natl. Acad. Sci. |
| | (UL115) | U.S.A. 112, 1767-1772 (2015). |
| | | Chandramouli et al. Structure of HCMV glycoprotein |
| | | B in the postfusion |
| | | conformation bound to a neutralizing human |
| | | antibody. Nat Commun. 2015 Sep. 14; 6: 8176, |
| | | Chandramouli et al. Structural basis for potent |
| | | antibody-mediated neutralization of human |
| | | cytomegalovirus Sci. Immunol. 2, eaan1457 (2017). |
| Lyme | Outer Surface | Ma et al, Safety, efficacy, and immunogenicity of a |
| | Protein A (OspA) | recombinant Osp subunit canine Lyme disease |
| | | vaccine. Volume 14, Issue 14, October 1996, Pages |
| | | 1366-1374 |
| Pertussis | Pertussis toxin (PT) | Seubert et al. Genetically detoxified pertussis toxin |
| | | (PT-91(/129G): implications for immunization and |
| | | vaccines. Expert Rev Vaccines. 2014 |
| | | October; 13 (10): 1191-204. doi: |
| | | 10.1586/14760584.2014.942641. Epub 2014 Sep. 3, |
| Dengue | E protein | Modis, Y., Ogata, S., Clements, D. & Harrison, S. C. |
| | | (2003) Proc. Natl. Acad. Sci. USA 100, 6986- |
| | | 6991. pmid: 12759475 |
| SARS | Spike (S) | Structure of SARS coronavinis spike receptor-binding |
| | glycoprotein | domain complexed with receptor. Science. 2005 Sep. |
| | | 16; 309(5742): 1864-8; WO2006068663A2 |
| MERS | Spike (S) | Immunogenicity and structures of a rationally |
| | glycoprotein | designed prefusion MERS-CoNT spike antnzen. PNAS |
| | | 2017 August., 114 (35) E7348-E7357. |
| | | https://doi.org/10.1073/pnas.1707304114 |
| Ebola | EBOV GP or sGP | Structures of Ebola virus GP and sGP in complex with |
| | [GP₁ and GP₂ | therapeutic antibodies. Nat Microbiol. 2016 Aug. |
| | subunits | 8; 1 (9): 16128. doi: 10.1038/mnicrobiol.2016.128. |
| Marberg | Marbera GP or | Hashiguchi et al. Structural basis for Marburg virus |
| | sGP | neutralization by a cross-reactive human antibody. |
| | | Cell. 2015 Feb. 26; 160 (5): 904-912. |
| Hantaan virus | Gn and Gc | Hantavirus Gn and Ge Envelope Glycoproteins: Key |
| | envelope | Structural Units for Virus Cell Entry and Virus |
| | glycoproteins | Assembly. Viruses. 2014 April; 6 (4): 1801-1822. |
| Hepatitis B | HepB surface | Raldao et al. Virus-like particles in vaccine |
| | antigen (HBs) | development. Expert Rev Vaccines. 2010 |
| | | October; 9 (10): 1149-76. |
| Measles | H and F proteins | Lobanova et al. The recombinant globular head |
| | | domain of the measles virus hemagglutinin protein as |
| | | a subunit vaccine against measles. Vaccine. 2012 Apr. |
| | | 26; 30 (20): 3061-7. |
| Nipah virus | G and F protein | Satterfield et al. Status of vaccine research and |
| | | development of vaccines for Nipah virus. Vaccine. |
| | | 34 (26): 2971-2975 (2016). |
| Rotatvirus | VP4 and VP8 | O'Ryan et al. Parenteral protein-based rotavirus |
| | | vaccine. Lancet Infectious Disease. 17(8): 786-787 |
| | | (2017). |
| Human | G and F proteins | Aertes et al. Adjuvant effect of the human |
| Metapneumo | | metapneumovirus (HMPV) matrix protein in HMPV |
| virus | | subunit vaccines. J Gen Virol. 2015 April; 96 (Pt 4): |
| | | 767-74; US 20180008697 A1. |
| Parainfluenza | HN and F proteins | Morein et al. Protein subunit vaccines of parainfluenza |
| virus | | type 3 virus: immunogenic effect in lambs and mice. J |
| | | Gen Virol. 1983 July; 64 (Pt 7): 1557-69. |
| Zika | Zika envelope | Recurrent Potent Human Neutralizing Antibodies to |
| | domain III | Zika Virus in Brazil and Mexico. Cell. 2017 May |
| | (ZEDIII) | 4; 169(4): 597-609.e 1 1. doi: |
| | | 10.1016/j.cell.2017.04.024. |
| Malaria | Pfs25, | Lee et al. Assessment of Pfs25 expressed from |
| | circumsporozoite | multiple soluble expression platforms for use as |
| | protein (CSP) | transmission-blockirw, vaccine candidates. Malar J. |
| | | 2016; 15: 405. |
| | | Plassmeyer et al. Structure of the |
| | | Plasmodium falciparum circumsporozoite protein, a |
| | | leading malaria vaccine candidate. J Biol Chem. 2009 |
| | | Sep. 25; 284 (39): 26951-63, |
| MenB | fHbp, NadA and | Davide et al. The new multicomponent vaccine against |
| | N-HBA | meningococcal serogroup B, 4CMenB: |
| | | immunological, functional and structural |
| | | characterization of the antigens. Vaccine. 2012 May |
| | | 30; 30 (0 2): B87-B97. |
| MenA, C. W- | oligosaccharide | Tontini et al. Comparison of CRM197, diphtheria |
| 135, and Y | | toxoid and tetanus toxoid as protein carriers for |
| | | meningococcal alycoconjugate vaccines. Vaccine. |
| | | 2013 Oct. 1; 31 (42): 4827-33. |

In some embodiments, the antigen is an antigenic protein is selected from a polypeptide of SEQ ID NOs: 52-88 and 90-113 or a variant thereof, as provided in Table 3.

TABLE 3
Non-Limiting List of Antigen Sequences

| | | SEQ |
|---|---|---|
| | | ID |
| Antigen | Amino Acid Sequence (UniPmot) | NO |
| Human immunodeficiency virus | >tr\|A0A1C9TBY8\|A0A1C9TBY8_9HIV1 Envelope | 52 |
| 1 (HIV-1) | glycoprotein gp160 OS = Human immunodeficiency | |
| gp160 | virus 1 GN = env PE = 3 SV = 1 | |
| | MRVKGIKKNYQHWWRGGIMLMGMLMICSSAEKLWVTVYYGVPVW | |
| | KEATTTLFCASDAKAQNPEMHNIWATHACVPTDPNPQEVILKNL | |
| | TEEFNMWKNNMVEQMHEDIISLWDQSLKPCVKLTPLCVTLNCTN | |
| | AESLNCTATNGTNNCSASTKPMEEMKNCSFNITTSVQDKKQQEY | |
| | ALFYKLDIIPIDNNENDLNNTNYTSYRLISCNTSVITQACPKIT | |
| | FEPIPIHYCAPAGFAILKCKDKRFNGTCPCKNVSTVQCTHGIRF | |
| | VVSTQLLLNGSLAEEGVVLRSENFTDNAKNIIVQLKDPVNITCT | |
| | RPNNNTRKSITIGPGRAFYATGQVIGDIRKAHCDLNGTEWDNAL | |
| | KQIVEELRKQYGNNITIFNSSSGGDPEIVMHSFNCGGEFFYCNT | |
| | AQLFNSTWLFNSTWNSTERLGNDTERTNDTITLPCKIKQVINMW | |
| | QTVGKAMYAPPIRGLIRCSSNITGLILTRDGSGNTTGNETFRPG | |
| | GGNMKDNWRSELYKYKVVKIEPLGVAPTRAKRRVVQREKRAAGL | |
| | GALFLGFLGMAGSTMGAASLTLTVQARQLLSGIVQQQNNLLRAI | |
| | EAQQHLLQLTVWGIKQLQARVLAVERYLRDQQLLGIWGCSGKLI | |
| | CTTTVPWNASWSNKSLDNIWENMTWMQWEKEIDNYTDVIYKLLE | |
| | ESQNQQEKNEQELLELDKWASLWNWFDITRMLWYIKIFIMIVGG | |
| | LVGLRIVFAVLSIVNRVRQGYSPLSFQTLFPAPRGPDRPEGTEE | |
| | GGGERGRDSSDRSAHGFLALIWGDLWSLCLFSYRRLRDLLLIAA | |
| | RIVELLGRRGWEVLKYWWSLLQYWSQELKKSAVSLLNATAIAVA | |
| | EGTDRIIEIVQRAGRAIIHIPRRIRQGAERALL | |
| Human immunodeficiency virus | gp120>tr\|A0A1C9TBY8\|33-524 | 53 |
| 1 (HIV-1) | LWVTVYYGVPVWKEATTTLFCASDAKAQNPEMHNIWATHACYPT | |
| gp120 | DPNPQEVILKNLTEEFNMWKNNMVEQWHEDIISLWDQSLKPCVK | |
| | LTPLCVTLNCTNAESLNCTATNGTNNCSASTKPMEEMKNCSFNI | |
| | TTSVQDKKQQEYALFYKLDIIPIDNNENDLNNTNYTSYRLISCN | |
| | TSVITQACPKITFEPIPIHYCAPAGFAILKCKDKRFNGTGPCKN | |
| | VSTVQCTHGIRPVVSTQLLLNGSLAEEGVVLRSENFTDNAKNII | |
| | VQLKDPVNITCTRPNNNTRKSITIGPGRAFYATGQVIGDIRKAH | |
| | CDLNGTEWDNALKQIVEELRKQYGNNITIFNSSSGGDPEIVMHS | |
| | FNCGGEFFYCNTAQLFNSTWLFNSTWNSTERLGNDTERTNDTIT | |
| | LPCKIKQVINMWQTVGKAMYAPPIRGLIRCSSNITGLILTRDGS | |
| | GNTTGNETFRPGGGNMKDNWRSELYKYKVVKIEPLGVAPTRAKR | |
| | RVVQREKR | |
| Human immunodeficiency virus | gp41>tr\|A0A1C9TBY8\|543-733 | 54 |
| 1 (HIV-1) | MGAASLTLTVQARQLLSGIVQQQNNLLRAIEAQQHLLQLTVWGI | |
| gp41 | KQLQARVLAVERYLRDQQLLGIWGCSGKLICTTTVPWNASWSNK | |
| | SLDNIWENMTWMQWEKEIDNYTDVIYKLLEESQNQQEKNEQELL | |
| | ELDKWASLWNWFDITRWLWYIKIFIMIVGGLVGLRIVFAVLSIV | |
| | NRVRQGYSPLSFQTL | |
| Human immunodeficiency virus | >tr\|A0A1C9TBY8\|675-696 | 55 |
| 1 (HIV-1) | ELDKWASLWNWFDITRWLWYIK | |
| MPER | | |
| Respiratory syncytial virus | >tr\|X4Y973\|X4Y973_9MONO Fusion glycoprotein | 56 |
| (RSV) type A | F0 OS = Respiratory syncytial virus type A | |
| F protein | GN = F PE = 3 SV = 1 | |
| | MELPILKTNAITTILAAVTLCFASSQNITEEFYQSTCSAVSKGY | |
| | LSALRTGWYTSVITIELSNIKENKCNGTDAKVKLIKQELDKYKN | |
| | AVTELQLLMQSTPAANNRARRELPRFMNYTLNNTKNNNVTLSKK | |
| | RKRRFLGFLLGVGSAIASGIAVSKVLHLEGEVNKIKNALLSTNK | |
| | AVVSLSNGVSVLTSKVLDLKNYIDKQLLPIVNKQSCSISNIETV | |
| | IEFQQKNNRLLEITREFSVNAGVTTPVSTYMLTNSELLSLINDM | |
| | PITNDQKKLMSNNVQIVRQQSYSIMSIIKEEVLAYVVQLPLYGV | |
| | IDTPCWKLHTSPLCTTNTKEGSNICLTRTDRGWYCDNAGSVSFF | |
| | PQAETCKVQSNRVFCDTMNSLTLPSEVNLCNIDIFNPKYDCKIM | |
| | TSKTDVSSSVITSLGAIVSCYGKTKCTASNKNRGIIKTFSNGCD | |
| | YVSNKGVDTVSVGNTLYYVNIQEGKSLYVKGEPIINFYDPLVFP | |
| | SDEFDASISQVNEKINQSLAFIRKSDELLHNVNVGKSTTNIMIT | |
| | TIIIVIIVILLLLIAVGLFLYCKARSTPVTLSKDQLSGINNIAF | |
| | SN | |
| Influenza A virus | >tr\|C3W5X2\|C3W5X2_9INFA Hemagglutinin | 57 |
| HA | OS = Influenza A virus | |
| | (A/Californda/07/2009(H1N1)) GN = HA PE = 1 SV = 1 | |
| | MKAILVVLLYTFATANADTLCIGYHANNSTDTVDTVLEKNVTVT | |
| | HSVNLLEDKHNGKLCKLRGVAPLHLGKCNIAGWILGNPECESLS | |
| | TASSWSYIVETPSSDNGTCYPGDFIDYEELREQLSSVSSFERFE | |
| | IFPKTSSWPNHDSNKGVTAACPRAGAKSFYKNLIWLVKKGNSYP | |
| | KLSKSYINDKGKEVLVLWGIHHPSTSADQQSLYQNADAYVFVGS | |
| | SRYSKKFKPEIAIRPKVRDQEGRMNYYWTLVEPGDKITFEATGN | |
| | LVVPRYAFAMERNAGSGIIISDTPVHDCNTTCQTPKGAINTSLP | |
| | FQNIHPITIGKCPKYVKSTKLRLATGLRNIPSIQSRGLFGAIAG | |
| | FIEGGWTGMVDGWYGYHHQNEQGSGYAADLKSTQNAIDEITNKV | |
| | NSVIEKMNTQFTAVGKEFNHLEKRIENLNKKVDDGFLDIWTYNA | |
| | ELLVLLENERTLDYHDSNVKNLYEKVRSQLKNNAKEIGNGCFEF | |
| | YHKCDNTCMESVKNGTYDYPKYSEEAKLNREEIDGVKLESTRIY | |
| | QILAIYSTVASSLVLVVSLGAISFWMCSNGSLQCRICI | |
| Influensa B virus | >tr\|A0A140EM53\|A0A140EM53_9INFB | 58 |
| HA | Hemagglutinin OS = Influenza B virus | |
| | (B/Victoria/809/2012) GN = HA PE = 3 SV = 1 | |
| | MKAIIVLLMVVTSNADRICTGITSSNSPHVVKTATQGEVNVTGV | |
| | IPLTTTPTKSYFANLKGTKTRGKLCPDCLNCTDLDVALGRPMCV | |
| | GTTPSAKASILHEVRPVTSGCFPIMHDRTKIRQLANLLRGYENI | |
| | RLSTQNVIDAEKAPGGPYRLGTSGSCPNATSKSGFFATMAKAVP | |
| | KDNNKNATNPLTVEVPYICAEGEDQITVWGFHSDNKTQMKNLYG | |
| | DSNPQKFTSSANGVTTHYVSQIGGFPDQTEDGGLPQSGRIVVDY | |
| | MMQKPGKTGTIVYQRGVLLPQKVWCASGRSKVIKGSLPLIGEAD | |
| | CLHEKYGGLNKSKPYYTGEHAKAIGNCPIWVKTPLKLANGTKYR | |
| | PPAKLLKERGFFGAIAGFLEGGWEGMIAGWHGYTSHGAHGVAVA | |
| | ADLKSTQEAINKITKNLNSLSELEVKNLQRLSGAMDELHNEILE | |
| | LDEKVDDLRADTISSQIELAVLLSNEGIINSEDEHLLALERKLK | |
| | KMLGPSAVDIGNGCFETKHKCNQTCLDRIAAGTFNAGEFSLPTF | |
| | DSLNITAASLNDDGLDNHTILLYYSTAASSLAVTLMLAIFIVYM | |
| | VSRDNVECSICL | |
| Epstein-Barr virus (EBV) | >sp\|P03200\|GP350_EBVB9 Envelope glycoprotein | 59 |
| glycoprotein 350/220 (gp350) | GP350 OS = Epstein-Barr virus (strain B95-8) | |
| | GN = BLLF1 PE = 1 SV = 1 | |
| | MEALLVCQYTIQSLIHLTGEDPGFFNVEIPEFPFYPTCNVCYTA | |
| | DVNVTINFDVGGKKHQLDLDFGQLTPHTKAVYQPRGAFGGSENA | |
| | TNLFLLELLGAGELALTMRSKKLPINVTTGEEQQVSLESVDVYF | |
| | QDVFGTMWCHHAEMQNPVYLIPETVPYIKWDNCNSTNITAVVRA | |
| | QGLDVTLPLSLPTSAQDSNFSVKTEMLGNEIDIECIMEDGEISQ | |
| | VLPGDNKFNITCSGYESHVPSGGILTSTSPVATPIPGTGYAYSL | |
| | RLTPRPVSRFLGNNSILYVFYSGNGPKASGGDYCIQSNIVFSDE | |
| | IPASQDMPTNTTDITYVGDNATYSVPMVTSEDANSPNVTVTAFW | |
| | AWPNNTETDEKCKWTLTSGTPSGCENISGAFASNRTFDITVSGL | |
| | GTAPKTLIITRTATNATTTTHKVIFSKAPESTTTSPTLNTTGFA | |
| | DPNTTTGLPSSTHVPTNLTAPASTGPTVSTADVTSPTPAGTTSG | |
| | ASPVTPSPSPWDNGTESKAPDMTSSTSPVTTPTPNATSPTPAVT | |
| | TPTPNATSPTPAVTTPTPNATSPTLGKTSPTSAVTTPTPNATSP | |
| | TLGKTSPTSAVTTPTPNATSPTLGKTSPTSAVTTPTPNATGPTV | |
| | GETSPQANATNHTLGGTSPTPVVTSQPKNATSAVTTGQHNITSS | |
| | STSSMSLRPSSNPETLSPSTSDNSTSHMPLLTSAHPTGGENITQ | |
| | VTPASISTHHVSTSSPAPRPGTTSQASGPGNSSTSTKPGEVNVT | |
| | KGTPPQNATSPQAPSGQKTAVPTVTSTGGKANSTTGGKHTTGHG | |
| | ARTSTEPTTDYGGDSTTPRPRYNATTYLPPSTSSKLRPRWTFTS | |
| | PPVTTAQATVPVPPTSQPRFSNLSMLVLQWASLAVLTLLLLLVM | |
| | ADCAFRRNLSTSHTYTTPPYDDAETYV | |
| Human cytomegalovirus | >sp\|P06473\|GB_HCMVA Envelope glycoprotein B | 60 |
| gB | OS = Human cytomegalovirus (strain AD169) | |
| | GN = gB PE = 1 SV = 1 | |
| | MESRIWCLVVCVNLCIVCLGAAVSSSSTSHATSSTHNGSHTSRT | |
| | TSAQTRSVYSQHVTSSEAVSHRANETIYNTTLKYGDVVGVNTTK | |
| | YPYRVCSMAQGTDLIRFEPNIICTSMKPINEDLDEGIMVVYKRN | |
| | IVAHTFKVRVYQKVLTFRRSYAYIYTTYLLGSNTEYVAPPMWEI | |
| | HHINKFAQCYSSYSRVIGGTVFVAYHRDSYENKTMQLIPDDYSN | |
| | THSTRYVTVKDQWHSRGSTWLYRETCNLNCMLTITTARSKYPYH | |
| | FFATSTGDVVYISPFYNGTNRNASYFGENADKFFIFPNYTIVSD | |
| | FGRPNAAPETHRLVAFLERADSVISWDIQDEKNVTCQLTFWEAS | |
| | ERTIRSEAEDSYHFSSAKMTATFLSKKQEVNMSDSALDCVRDEA | |
| | INKLQQIFNTSYNQTYEKYGNVSVFETSGGLVVFWQGIKQKSLV | |
| | ELERLANRSSLNITHRTRRSTSDNNTTHLSSMESVHNLVYAQLQ | |
| | FTYDTLRGYINRALAQIAEAWCVDQRRTLEVFKELSKINPSAIL | |
| | SAIYNKPIAARFMGDVLGLASCVTINQTSVKVLRDMNVKESPGR | |
| | CYSRPVVIFNFANSSYVQYGQLGEDNEILLGNHRTEECQLPSLK | |
| | IFIAGNSAYEYVDYLFKRMIDLSSISTVDSMIALDIDPLENTDF | |
| | RVLELYSQKELRSSNVFDLEEIMREFNSYKORVKYVEDKVVDPL | |
| | PPYLKGLDDLMSGLGAAGKAVGVAIGAVGGAVASVVEGVATFLK | |
| | NPEGAFTIILVAIAVVIITYLIYTRQRRLCTQPLQNLFPYLVSA | |
| | DGTTVTSGSTKDTSLQAPPSYEESVYNSGRKGPGPPSSDASTAA | |
| | PPYTNEQAYQMLLALARLDAEQRAQQNGTDSLDGQTGTQDKGQK | |
| | PNLLDRLRHRKNGYRHLKDSDEEENV | |
| Human cytomegalovirus | >sp\|P16837\|UL128_HCMVA Uncharacterized | 61 |
| UL128 | protein UL128 OS = Human cytomegalovirus | |
| | (strain AD169) GN = UL128 PE = 1 SV = 2 | |
| | MSPKDLTPFLTTLWLLLGHSRVPRVRAEECCEFINVNHPPERCY | |
| | DFKMCNRFTVALRCPDGEVCYSPEKTAEIRGIVTTMTHSLTRQV | |
| | VHNKLTSCNYNPLYLEADGRIRCGKVNDKAQYLLGAAGSVPYRW | |
| | INLEYDKITRIVGLDQYLESVKKHKRLDVCRAKMGYMLQ | |
| Human cytomegalovirus | >sp\|F5HCP3\|UL130_HCMVM Envelope glycoprotein | 62 |
| UL130 | UL130 OS = Human cytomegalovirus (strain | |
| | Merlin) GN = UL130 PE = 1 SV = 1 | |
| | MLRLLLRHHFHCLLLCAVWATPCLASPWSTLTANQNPSPPWSKL | |
| | TYSKPHDAATFYCPFLYPSPPRSPLQFSGFQRVSTGPECRNETL | |
| | YLLYNREGQTLVERSSTWVKKVIWYLSGRNQTILQRMPRTASKP | |
| | SDGNVQISVEDAKIFGAHMVPKQTKLLRFVVNDGTRYQMCVMKL | |
| | ESWAHVFRDYSVSFQVRLTFTEANNQTYTFCTHPNLIV | |
| Human cytomegalovirus | >sp\|HET4\|U131_HCMVM Protein UL131A | 63 |
| UL131A | OS = Human cytomegalovirus (strain Merlin) | |
| | GN = UL131A PE = 1 SV = 1 | |
| | MRLCRVWLSVCLCAVVLGQCQRETAEKNDYYRVPHYWDACSRAL | |
| | PDQTRYKYVEQLVDLTLNYHYDASHGLDNFDVLKRINVTEVSLL | |
| | ISDFRRQNRRGGTNKRTTFNAAGSLAPHARSLEFSVRLFAN | |
| Human cytomegalovirus | >sp\|P12824\|GH_HCMVA Envelope glycoprotein H | 64 |
| gH (UL75) | OS = Human cytomegalovirus (strain AD169) | |
| | GN = gH PE = 1 SV = 1 | |
| | MRPGLPPYLTVFTVYLLSHLPSQRYGADAASEALDPHAFHLLLN | |
| | TYGRPIRFLRENTTQCTYNSSLRNSTVVRENAISFNFFQSYNQY | |
| | YVFHMPRCLFAGPLAEQFLNQVDLTETLERYQQRLNTYALVSKD | |
| | LASYRSFSQQLKAQDSLGQQPTTVPPPIDLSIPHVWMPPQTTPH | |
| | DWKGSHTTSGLHRPHFNQTCILFDGHDLLFSTVTPCLHQGFYLM | |
| | DELRYVKITLTEDFFVVTVSIDDDTPMLLIFGHLPRVLFKAPYQ | |
| | RDNFILRQTEKHELLVLVKKAQLNRHSYLKDSDFLDAALDFNYL | |
| | DLSALLRNSFHRYAVDVLKSGPCQMLDRRTVEMAFAYALALFAA | |
| | ARQEEAGTEISIPRALDRQAALLQIQEFMITCLSQTPPRTTLLL | |
| | YPTAVDLAKRALWTPDQITDITSLVRLVYILSKQNQQHLIPQWA | |
| | LRQIADFALQLHKTHLASFLSAFARQELYLMGSLVHSMLVHTTE | |
| | RREIFIVETGLCSLAELSHFTQLLAHPHHEYLSDLYTPCSSSGR | |
| | RDHSLERLTRLFPDATVPATVPAALSILSTMQPSTLETFPDLFC | |
| | LPLGESFSALTVSEHVSYVVTNQYLIKGISYPVSTTVVGQSLII | |
| | TQTDSQTKCELTRNMHTTHSITAALNISLENCAFCQSALLEYDD | |
| | TQGVINIMYMHDSDDVLFALDPYNEVVVSSPRTHYLMLLKNGTV | |
| | LEVTDVVVDATDSRLLMMSVYALSAIIGIYLLYRMLKTC | |
| Human cytomegalovirus | >sp\|P16832\|GL_HVMVA Envelope glycoprotein L | 65 |
| gL (UL115) | OS = Human cytomegalovirus (strain AD169) | |
| | GN = gL PE = 1 SV = 2 | |
| | MCPRPDCGFSFSPGPVVLLWCCLLLPIVSSVAVSVAPTAAEKVP | |
| | AECPELTRRCLLGEVFQGDKYESWLRPLVNVTRRDGPLSQLIRY | |
| | RPVTPEAANSVLLDDAFLDTLALLYNNPDQLRALLTLLSSDTAP | |
| | RWMTVMRGYSECGDGSPAVYTCVDDLCRGYDLTRLSYGRSIFTE | |
| | HVLGFELVPPSLFNVVVAIRNEATRTNRAVRLPVSTAAAPEGIT | |
| | LFYGLYNAVKEFCLRHQLDPPLLRHLDKYYAGLPPELKQTRVNL | |
| | PAHSRYGPQAVDAR | |
| Lyme | >sp\|Q04968\|OSPA7_BORBG Outer surface protein | 66 |
| Outer Surface Protein A | A OS = Borreliella burgdorferi GN = ospA PE = 3 | |
| (OspA) | SV = 1 | |
| | MKKYLLGIGLILALIACKQNVSSLDEKNSVSVDVPGGMKVLVSK | |
| | EKNKDGKYDLMATVDNVDLKGTSDKNNGSGILEGVKADKSKVKL | |
| | TVADDLSKTTLEVLKEDGTVVSRKVTSKDKSTTEAKFNEKGELS | |
| | EKTMTRANGTTLEYSQMTNEDNAAKAVETLKNGIKFEGNLASGK | |
| | TAVEIKEGTVTLKREIDKNGKVTVSLNDTASGSKKTASWQESTS | |
| | TLTISANSKKTKDLVFLTNGTITVQNYDSAGTKLEGSAAEIKKL | |
| | DELKNALR | |
| Bordetella pertussis | >sp\|P04977\|TOX1_BORPE Pertussis toxin | 67 |
| Pertussis toxin (PT) subunits | subunit 1 OS = 2 Bordetella pertussis (strain | |
| 1-5 | Tohama I/ATCC BAA-589/NCTC 13251) | |
| | GN = ptxa PE = 1 SV = 1 | |
| | MRCTRAIRQTARTGWLTWLAILAVTAPVTSPAWADDPPATVYRY | |
| | DSRPPEDVFQNGFTAWGNNDNVLDHLTGRSCQVGSSNSAFVSTS | |
| | SSRRYTEVYLEHRMQEAVEAERAGRGTGHFIGYIYEVRADNNFY | |
| | GAASSYFEYVDTYGDNAGRILAGALATYQSEYLAHRRIPPENIR | |
| | RVTRVYHNGITGETTTTEYSNARYVSQQTRANPNPYTSRRSVAS | |
| | IVGTLVRMAPVIGACMARQAESSEAMAAWSERAGEAMVLVYYES | |
| | IAYSF | |
| Dengue virus | >sp\|P17763\|281-775 | 68 |
| Envelope protein E | MRCVGIGNRDFVEGLSGATWVDVVLEHGSCVTTMAKDKPTLDIE | |
| | LLKTEVTNPAVLRKLCIEAKISNTTTDSRCPTQGEATLVEEQDT | |
| | NFVCRRTFVDRGWGNGCGLFGKGSLITCAKFKCVTKLEGKIVQY | |
| | ENLKYSVIVTVHTGDQHQVGNETTEHGTTATITPQAPTSEIQLT | |
| | DYGALTLDCSPRTGLDENEMVLLTMEKKSWLVHKQWFLDLPLPW | |
| | TSGASTSQETWNRQDLLVTFKTAHAKKQEVVVLGSQEGAMHTAL | |
| | TGATEIQTSGTTTIFAGHLKCRLKMDKLTLKGMSYVMCTGSFKL | |
| | EKEVAETQHGTVLVQVKYEGTDAPCKIPFSSQDEKGVTQNGRLI | |
| | TANPIVTDKEKPVNIEAEPPFGESYIVVGAGEKALKLSWFKKGS | |
| | SIGKMFEATARGARRMAILGDTAWDFGSIGGVFTSVGKLIHQIF | |
| | GTAYGVLFSGVSWTMKIGIGILLTWLGLNSRSTSLSMTCIAVGM | |
| | VTLYLGVMVQA | |
| HUMAN SARS coronavirus (SARS) | >sp\|P59594\|SPIKE_CVHSA Spike glycoprotein | 69 |
| Spike (S) glycoprotein | OS = Human SAPS coronavirus GN = S PE = 1 SV = 1 | |
| | MFIFLLFLTLTSGSDLDRCTTFDDVQAPNYTQHTSSMRGVYYPD | |
| | EIFRSDTLYLTQDLFLPFYSNVTGFHTINHTFGNPVIPFKDGIY | |
| | FAATEKSNVVRGWVFGSTMNNKSQSVIIINNSTNVVIRACNFEL | |
| | CDNPFFAVSKPMGTQTHTMIFDNAFNCTFEYISDAFSLDVSEKS | |
| | GNFKHLREFVFKNKDGFLYVYKGYQPIDVVRDLPSGFNTLKPIF | |
| | KLPLGINITNFRAILTAFSPAQDIWGTSAAAYFVGYLKPTTFML | |
| | KYDENGTITDAVDCSQNPLAELKCSVKSFEIDKGIYQTSNFRVV | |
| | PSGDVVRFPNITNLCPFGEVFNATKFPSVYAWERKKISNCVADY | |
| | SVLYNSTFFSTFKCYGVSATKLNDLCFSNVYADSFVVKGDDVRQ | |
| | IAPGQTGVIADYNYKLPDDFMGCVLAWNTRNIDATSTGNYNYKY | |
| | RYLRHGKLRPFERDISNVPFSPDGKPCTPPALNCYWPLNDYGFY | |
| | TTTGIGYQPYRVVVLSFELLNAPATVCGPKLSTDLIKNQCVNFN | |
| | FNGLTGTGVLTPSSKRFQPFQQFGRDVSDFTDSVRDPKTSEILD | |
| | ISPCSFGCVSVITPCTNASSEVAVLYQDVNCTDVSTAIHADQLT | |
| | PAWRIYSTGNNVFQTQAGCLIGAEHVDTSYECDIPIGAGICASY | |
| | HTVSLLRSTSQKSIVAYTMSLGADSSIAYSNNTIAIPTNFSISI | |
| | TTEVMPVSMAKTSVDCNMYICGDSTECANLLLQYGSFCTQLNRA | |
| | LSGIAAEQDRNTREVFAQVKQMYKTPTLKYFGGFNFSQILPDPL | |
| | KPTKRSFIEDLLENKVTLADAGFMKQYGECLGDINARDLICAQK | |
| | FNGLTVLPPLLTDDMIAAYTAALVSGTATAGWTFGAGAALQIPF | |
| | AMQMAYRFNGIGVTQNVLYENQKQIANQFNKAISQIQESLTTTS | |
| | TALGKLQDVVKQNAQALNTLVKQLSSNFGAISSVLNDILSRLDK | |
| | VEAEVQIDRLITGRLQSLQTYVTQQLIRAAEIRASANLAATKMS | |
| | ECVLGQSKRVDFCGKGYHLMSFPQAAPHGVVFLHVTYVPSQERN | |
| | FTTAPAICHEGKAYFPREGVFVFNGTSWFITQRNFFSPQIITTD | |
| | NTFVSGNCDVVIGIINNTVYDPLQPELDSFKEELDKYFKNHTSP | |
| | DVDLGDISGINASVVNIQKEIDRLNEVAKNLNESLIDLQELGKY | |
| | EQYIKWPWYVWLGFIAGLIAIVMVTILLCCMTSCCSCLKGACSC | |
| | GSCCKFDEDDSEPVLKGVKLHYT | |
| Middle East respiratory | >tr\|R9UCW7\|R9UCW7_9BETC Spike glycoprotein | 70 |
| syndrome-related coronavirus | OS = Middle East respiratory syndrome-related | |
| (MERS) | coronavirus PE = 4 SV = 1 | |
| Spike (S) glycoprotein | MIHSVFLLMFLLTPTESYVDVGPDSIKSACIEVDIQQTFFDKTW | |
| | PRPIDVSKADGIIYPQGRTYSNITITYQGLFPYQGDHGDMYVYS | |
| | AGHATGTTPQKLFVANYSQDVKQFANGFVVRIGAAANSTGTVII | |
| | SPSTSATIRKIYPAFMLGSSVGNFSDGKMGRFFNHTLVLLPDGC | |
| | GTLLRAFYCILEPRSGNHCPAGNSYTSFATYHTPATDCSDGNYN | |
| | RNASLNSFKEYFNLRNCTFMYTYNITEDEILEWFGITQTAQGVH | |
| | LFSSRYVDLYGGNMEQFATLPVYDTIKYYSIIPHSIRSIQSDRK | |
| | AWAAFYVYKLQPLTFLLDFSVDGYIRRAIDCGFNDLSQLHCSYE | |
| | SFDVESGVYSVSSFEAKPSGSVVEQAEGVECDFSPLISGTPPQV | |
| | YNFKRLVFTNCNYNLTKLLSLFSVNDFTCSQISPAAIASNCYSS | |
| | LILDYFSYPLSMKSDLSVSSAGPISQFNYKQSFSNPTCLILATV | |
| | PHNLTTITKPLKYSYINKCSRLLSDDRTEVPQLVNANQYSPCVS | |
| | IVPSTVWEDGDYYRKQLSPLEGGGWLVASGSTVAMTEQLQMGFG | |
| | ITVQYGTDTNSVCPKLEFANDTKIASQLGNCVEYSLIGVSGRGV | |
| | FQNCTAVGVRQQRFVYDAYQNLVGYYSDDGNYYCLRACVSVPVS | |
| | VIYDKETKTHATLFGSVACEHISSTMSQYSRSTPSMLKRRDSTY | |
| | GPLQTPVGCVLGLVNSSLFVEDCKLPLGQSLCALPDTPSTLTPR | |
| | SVRSVPGEMRLASIAFNHPIQVDQLNSSYFKLSIPTNFSFGVTQ | |
| | EYIQTTIQKVTVDCKQYVCNGTQKCEQLLREYGQFCSKINQALH | |
| | GANLRQDDSVRNLFASVKSSQSSPIIPGFGGDFNLTLLEPVSIS | |
| | TGSRSARSAIEDLLEDKVTIADPGYMQGYDDCMQQGPASARDLI | |
| | CAQYVAGYKVLPPLMDVNMEAAYTSSLLGSIAGVGWTAGLSSFA | |
| | AIPFAQSIFYRLNGVGITQQVLSENQKLIANKFNQALGAMQTGF | |
| | TTTNEAFHKVQDAVNNNAQALSKLASELSNTFGAISASIGDIIQ | |
| | RLDVLEQDAQIDRLINGRLTTLNAFVAQQLVRSESAALSAQLAK | |
| | DKVNECVKAQSKRSGFCGQGTHIVSFVVNAPNGLYFMHVGYYPS | |
| | NHIEVVSAYGLCDAANPTNCIAPVNGYFIKTNNTRIVDEWSYTG | |
| | SSFYAPEPITSLNTKYVAPQVTYQNISTNLPPPLLGNSTGIDFQ | |
| | DELDEFFKNVSTSIPNFGSLQINTTLLDLTYEMLSLSQQVVKAL | |
| | NESYIDLKELGNYTYYNKPWYIWLGFIAGLVALALCVFFILCC | |
| | TGCGTNCMGKLKCNRCCDRYEEYDLEPHKVHVH | |
| Zaire ebolavirus | >sp\|Q05320\|VGP_EBOZM Envelope glycoprotein | 71 |
| GP | OS = Zaire ebolavirus (strain Mayinga-76) | |
| | GN = GP PE = 1 SV = 1 | |
| | MGVTGILQLPRDRFKRTSFFLWVIILFQRTFSIPLGVIHNSTLQ | |
| | VSDVDKLVCRDKLSSTNQLRSVGLNLEGNGVATDVPSATKRWGF | |
| | RSGVPPKVVNYEAGEWAENCYNLEIKKPDGSECLPAAPDGIRGF | |
| | PRCRYVHKVSGTGPCAGDFAFHKEGAFFLYDRLASTVIYRGTTF | |
| | AEGVVAFLILPQAKKDFFSSHPLREPVNATEDPSSGYYSTTIRY | |
| | QATGFGTNETEYLFEVDNLTYVQLESRFTPQFLLQLNETIYTSG | |
| | KRSNTTGKLIWKVNPEIDTTIGEWAFWETKKNLTRKIPSEELSF | |
| | TVVSNGAKNISGQSPARTSSDPGTNTTTEDHKIMASENSSAMVQ | |
| | VHSQGREAAVSHLTTLATISTSPQSLTTKPGPDNSTHNTPVYKL | |
| | DISEATQVEQHHRRTDNDSTASDTPSATTAAGPPKAENTNTSKS | |
| | TDFLDPATTTSPQNHSETAGNNNTHHQDTGEESASSGKLGLITN | |
| | TIAGVAGLITGGRRTRREAIVNAQPKCNPNLHYWTTQDEGAAIG | |
| | LAWIPYFGPAAEGIYIEGLMHNQDGLICGLRQLANETTQALQLF | |
| | LRATTELRTFSILNRKAIDFLLQRWGGTCHILGPDCCIEPHDWT | |
| | KNITDKIDQIIHDFVDKTLPDQGDNDNWWTGWRQWIPAGIGVTG | |
| | VIIAVIALFCICKFVF | |
| Marburg virus | >sp\|P35253\|VGP_MABVM Envelope glycoprotein | 72 |
| GP | OS = Lake Victoria marburgvirus (strain | |
| | Musoke-80) GN = GP PE = 1 SV = 1 | |
| | MKTTCFLISLILIQGTKNLPILEIASNNQPQNVDSVCSGTLQKT | |
| | EDVHLMGFTLSGQKVADSPLEASKRWAFRTGVPPKNVEYTECEE | |
| | AKTCYNISVTDPSGKSLLLDPPTNIRDYPKCKTIHHIQGQNPHA | |
| | QGIALHLWGAFELYDRIASTTMYRGKVFTEGNIAAMIVNKTVHK | |
| | MIFSRQGQGYRHMNLTSTNKYWTSSNGTQTNDTGCFGALQEYNS | |
| | TKNQTCAPSKIPPPLPTARPEIKLTSTPTDATKLNTTDPSSDDE | |
| | DLATSGSGSGEREPHTTSDAVTKQGLSSTMPFTPSPQPSTPQQG | |
| | GNNTNHSQDAVTELDKNNTTAQPSMPPHNTTTISTNNTSKHNFS | |
| | TLSAPLQNTTNDNTQSTITENEQTSAPSITTLPPTGNPTTAKST | |
| | SSKKGPATTAPNTTNEHETSPPPTPSSTAQHLVYFRRKRSILWR | |
| | EGDMFPFLDGLINAPIDFDPVPNTKTIFDESSSSGASAEEDQHA | |
| | SPNISLTLSYFPNINENTAYSGENENDCDAELRIWSVQEDDLAA | |
| | GLSWIPFFGPGIEGLYTAVLIKNQNNLVCRLRRLANQTAKSLEL | |
| | LLRVTTEERTFSLINRHAIDFLLTRWGGTCKVLGPDCCIGIEDL | |
| | SKNISEQIDQIKKDEQKEGTGWGLGGKWWTSDWGVLTNLGILLL | |
| | LSIAVLIALSCICRIFTKYIG | |
| Hanta virus | >sp\|P08668\|19-648 | 73 |
| Gn envelope glycoprotein | LRNVYDMKIECPHTVSFGENSVIGYVELPPVPLADTAQMVPESS | |
| | CNMDNHQSLNTITKYTQVSWRGKADQSQSSQNSFETVSTEVDLK | |
| | GTCVLKHKMVEESYRSRKSVTCYDLSCNSTYCKPTLYMIVPIHA | |
| | CNMMKSCLIALGPYRVQVVYERSYCMTGVLIEGKCFVPDQSVVS | |
| | IIKHGIFDIASVHIVCFFVAVKGNTYKIFEQVKKSFESTCNDTE | |
| | NKVQGYYICIVGGNSAPIYVPTLDDFRSMEAFTGIFRSPHGEDH | |
| | DLAGEEIASYSIVGPANAKVPHSASSDTLSLIAYSGIPSYSSLS | |
| | ILTSSTEAKHVFSPGLEPKLNHTNCDKSAIPLIWTGMIDLPGYY | |
| | EAVHPCTVFCVLSGPGASCEAFSEGGIFNITSPMCLVSKQNRFR | |
| | LTEQQVNFVCQRVDMDIVVYCNGQRKVILTKTLVIGQCIYTITS | |
| | LFSLLPGVAHSIAVELCVPGFHGWATAALLVTFCFGWVLIPAIT | |
| | FIILTVLKFIANIFHTSNQENRLKSVLRKIKEEFEKTKGSMVCD | |
| | VCKYECETYKELKAHGVSCPQSQCPYCFTHCEPTEAAFQAHYKV | |
| | CQVTHRFRDDLKKTVTPQNFTPGCYRTLNLFRYKSRCYIFTMWI | |
| | FLLVLESILWAASA | |
| Hanta virus | >sp\|P08668\|649-1135 | 74 |
| Gc envelope glycoprotein | SETPLTPVWNDNAHGVGSVPMHTDLELDFSLTSSSKYTYRRKLT | |
| | NPLERAQSIDLHIEIEEQTIGVDVHALGHWFDGRLNLKTSFHCY | |
| | GACTKYEYPWHTAKCHYERDYQYETSWGCNPSDCPGVGTGCTAC | |
| | GLYLDQLKPVGSAYKIITIRYSRRVCVQFGEENLCKIIDMNDCF | |
| | VSRHVKVCIIGTVSKFSQGDTLLFFGPLEGGGLIFKHWCTSTCQ | |
| | FGDPGDIMSPRDKGFLCPEFPGSFRKKCNFATTPICEYDGNMVS | |
| | GYKKVMATIDSFQSFNTSTMHFTDERIEWKDPDGMLRDHINILV | |
| | TKDIDFDNLGENPCKIGLQTSSIEGAWGSGVGFTLTCLVSLTEC | |
| | PTFLTSIKACDKAICYGAESVTLTRGQNTVKVSGKGGHSGSTFP | |
| | CCHGEDCSQIGLHAAAPHLDKVNGISEIENSKVYDDGAPQCGIK | |
| | CWTVKSGEWISGIFSGNWIVLIVLCVFLLFSLVLLSILCPVRKH | |
| | KKS | |
| Hepatitis B | >tr\|Q9DIX1\|Q9DIX1_HBV Surface antigen HBsAg | 75 |
| HepB surface antigen (HBs) | OS = Hepatitis B virus GN = S PE = 4 SV = 1 | |
| | MENITSGFLGPLLVLQAGFFLLTKILTIPQSLNSWWTSLSFLGG | |
| | NTVCLGQNSQSPTSNHSPTSCPPTCPGYRWMCLRRFIIFLFILL | |
| | LCLIFLLVLLDYQGMLPVCPLIPGSSTTSTGPCRTCKTPAQGTS | |
| | MYPSCCCTKPSDGNCTCIPIPSSWAFGKFLWEWASARFSWLSLI | |
| | VPFVQWFVGLSPTVWLSVIWMMWYWGPSLYSILSPFLPLLPIFF | |
| | CLWVYI | |
| Measles | >sp\|P08362\|HEMA_MEASE Hemagglutinin | 76 |
| H protein | glycoprotein OS = Measles virus (strain | |
| | Edmonston) GN = H PE = 1 SV = 1 | |
| | MSPQRDRINAFYKDNPHPKGSRIVINREHLMIDRPYVLLAVLFV | |
| | MFLSLIGLLAIAGIRLHRAAIYTAEIHKSLSTNLDVTNSIEHQV | |
| | KDVLTPLFKIIGDEVGLRTPQRFTDLVKFISDKIKFLNPDREYD | |
| | FRDLTWCINPFERIKLDYDQYCADVAAEELMNALVNSTLLETRT | |
| | TNQFLAVSKGNCSGPTTIRGQFSNMSLSLLDLYLGRGYNVSSIV | |
| | TMTSQGMYGGTYLVEKPNLSSKRSELSQLSMYRVFEVGVIRNPG | |
| | LGAPVFHMTNYLEQPVSNDLSNCMVALGELKLAALCHGEDSITI | |
| | PYQGSGKGVSFQLVKLGVWKSPTDMQSWVPLSTDDPVIDRLYLS | |
| | SHRGVIADNQAKWAVPTTRTDDKLRMETCFQQACKGKIQALCEN | |
| | PEWAPLEDNRIPSYGVLSVDLSLTVELKIKIASGFGPLITHGSG | |
| | MDLYKSNHNNVYWLTIPPMKNLALGVINTLEWIPRFKVSPYLFN | |
| | VPIKEAGEDCHAPTYLPAEVDGDVKLSSNLVILPGQDLQYVLAT | |
| | YDTSRVEHAVVYYVYSPSRSFSYFYPFRLPIKGVPIELQVECET | |
| | WDQKLWCRHFCVLADSESGGHITHSGMEGMGVSCTVTREDGTNR | |
| | R | |
| Measles | >sp\|P69353\|FUS_MEASE Fusion glycoprotein F0 | 77 |
| F protein | OS = Measles virus (strain Edmonston) GN = F | |
| | PE = 3 SV = 1 | |
| | MGLKVNVSAIFMAVLLTLQTPTGQIHWGNLSKIGVVGIGSASYK | |
| | VMTRSSHQSLVIKLMPNITLLNNCTRVEIAEYRRLLRTVLEPIR | |
| | DALNAMTQNIRPVQSVASSRRHKRFAGVVLAGAALGVATAAQIT | |
| | AGIALHQSMLNSQAIDNLRASLETTNQAIEAIRQAGQEMILAVQ | |
| | GVQDYINNELIPSMNQLSCDLIGQKLGLKLLRYYTEILSLFGPS | |
| | LRDPISAEISIQALSYALGGDINKVLEKLGYSGGDLLGILESRG | |
| | IKARITHVDTESYFIVLSIAYPTLSEIKGVIVHRLEGVSYNIGS | |
| | QEWYTTVPKYVATQGYLISNFDESSCTFMPEGTVCSQNALYPMS | |
| | PLLQECLRGSTKSCARTLVSGSFGNRFILSQGNLIANCASILCK | |
| | CYTTGTIINQDPDKILTYIAADHCPVVEVNGVTIQVGSRRYPDA | |
| | VYLHRIDLGPPISLERLDVGTNLGNAIAKLEDAKELLESSDQIL | |
| | RSMKGLSSTSIVYILIAVCLGGLIGIPALICCCRGRCNKKGEQV | |
| | GMSRPGLKPDLTGTSKSYVRSL | |
| Zika | >sp\|Q32ZE1\|291-790 | 78 |
| Zika envelope domain III | IRCIGVSNRDFVEGMSGGTWVDVVLEHGGCVTVMAQDKPTVDIE | |
| (ZEDIII) | LVTTTVSNMAEVRSYCYEASISDMASDSRCPTQGEAYLDKQSDT | |
| | QYVCKRTLVDRGWGNGCGLFGKGSLVTCAKFTCSKKMTGKSIQP | |
| | ENLEYRIMLSVHGSQHSGMIGYETDEDRAKVEVTPNSPRAEATL | |
| | GGFGSLGLDCEPRTGLDFSDLYYLTMNNKHWLVHKEWFHDIPLP | |
| | WHAGADTGTPHWNNKEALVEFKDAHAKRQTVVVLGSQEGAVHTA | |
| | LAGALEAEMDGAKGRLFSGHLKCRLKMDKLRLKGVSYSLCTAAF | |
| | TFTKVPAETLHGTVTVEVQYAGTDGPCKIPVQMAVDMQTLTPVG | |
| | RLITANPVITESTENSKMMLELDPPFGDSYIVIGVGDKKITHHW | |
| | HRSGSTIGKAFEATVRGAKRMAVLGDTAWDFGSVGGVFNSLGKG | |
| | IHQIFGAAFKSLFGGMSWFSQILIGTLLVWLGLNTKNGSISLTC | |
| | LALGGVMIFLSTAVSA | |
| Malaria | >sp\|P08677\|CSP_PLAVB Circumsperozoite | 79 |
| circumsporozoite protein | protein OS = Plasmodium vivax (strain Belem) | |
| (CSP) | PE = 3 SV = 2 | |
| | MKNFILLAVSSILLVDLFPTHCGHNVDLSKAINLNGVNFNNVDA | |
| | SSLGAAHVGQSASPGRGLGENPDDEEGDAKKKKDGKKAEPKNPR | |
| | ENKLKQPGDRADGQPAGDRADGQPAGDRADGQPAGDRAAGQPAG | |
| | DRADGQPAGDRADGQPAGDRADGQPAGDRADGQPAGDRAAGQPA | |
| | GDRAAGQPAGDRADGQPAGDRAAGQPAGDRADGQPAGDRAAGQP | |
| | AGDRADGQPAGDRAAGQPAGDRAAGQPAGDRAAGQPAGDRAAGQ | |
| | PAGNGAGGQAAGGNAGGGQGQNNEGANAPNEKSVKEYLDKVRAT | |
| | VGTEWTPCSVTCGVGVRVRRRVNAANKKPEDLTLNDLETDVCTM | |
| | DKCAGIFNVVSNSLGLVILLVALFN | |
| Nipah virus | >sp\|Q9IH63\|FUS_NIPAV Fusion glycoprotein F0 | 80 |
| F protein | OS = Nipah virus GN = F PE = 1 SV = 1 | |
| | MVVILDKRCYCNLLILILMISECSVGILHYEKLSKIGLVKGVTR | |
| | KYKIKSNPLTKDIVIKMIPNVSNMSQCTGSVMENYKTRLNGILT | |
| | PIKGALEIYKNNTHDLVGDVRLAGVIMAGVAIGIATAAQITAGV | |
| | ALYEAMKNADNINKLKSSIESTNEAVVKLQETAEKTVYVLTALQ | |
| | DYINTNLVPTIDKISCKQTELSLDLALSKYLSDLLFVFGPNLQD | |
| | PVSNSMTIQAISQAFGGNYETLLRTLGYATEDFDDLLESDSITG | |
| | QIIYVDLSSYYIIVRVYFPILTEIQQAYIQELLPVSFNNDNSEW | |
| | ISIVPNFILVRNTLISNIEIGFCLITKRSVICNQDYATPMTNNM | |
| | RECLTGSTEKCPRELVVSSHVPRFALSNGVLFANCISVTCQCQT | |
| | TGRAISQSGEQTLLMIDNTTCPTAVLGNVIISLGKYLGSVNYNS | |
| | EGIAIGPPVFTDKVDISSQISSMNQSLQQSKDYIKEAQRLLDTV | |
| | NPSLISMLSMIILYVLSIASLCIGLITFISFIIVEKKRNTYSRL | |
| | EDRRVRPTSSGDLIYIGT | |
| Nipah virus | >sp\|Q9IH62\|GLYCP_NIPAV Glycoprotein G | 81 |
| G protein | OS = Nipah virus GN = G PE = 1 SV = 1 | |
| | MPAENKKVRFENTTSDKGKIPSKVIKSYYGTMDIKKINEGLLDS | |
| | KILSAFNTVIALLGSIVIIVMNIMIIQNYTRSTDNQAVIKDALQ | |
| | GIQQQIKGLADKIGTEIGPKVSLIDTSSTITIPANIGLLGSKIS | |
| | QSTASINENVNEKCKFTLPPLKIHECNISCPNPLPFREYRPQTE | |
| | GVSNLVGLPNNICLQKTSNQILKPKLISYTLPVVGQSGTCITDP | |
| | LLAMDEGYFAYSHLERIGSCSRGVSKQRIIGVGEVLDRGDEVPS | |
| | LFMTNVWTPPNPNTVYHCSAVYNNEFYYVLCAVSTVGDPILNST | |
| | YWSGSLMMTRLAVKPKSNGGGYNQHQLALRSIEKGRYDKVMPYG | |
| | PSGIKQGDTLYFPAVGFLVRTEFKYNDSNCPITKCQYSKPENCR | |
| | LSMGIRPNSHYILPSGLLKYNLSDGENPKVVFIEISDQRLSIGS | |
| | PSKIYDSLGQPVFYQASFSWDTMIKFGDVLTVNPLVVNWRNNTV | |
| | ISRPGQSQCPRFNTCPEICWEGVYNDAFLIDRINWISAGVFLDS | |
| | NQTAENPVFTVFKDNEILYRAQLASEDTNAQKTITNCFLLKNKI | |
| | WCISLVEIYDTGDNVIRPKLFAVKIPEQCT | |
| Rotavirus | >sp\|P11193\|VP4_ROTHW Outer capsid protein | 82 |
| VP4 protein | VP4 OS = Rotavirus A (strain RVA/Human/United | |
| | States/Wa/1974/G1P1A[8]) PE = 1 SV = 3 | |
| | MASLIYRQLLTNSYSVDLHDEIEQIGSEKTQNVTINFSPFAQTR | |
| | YAPVNWGHGEINDSTTVEPILDGPYQPTTFTPPNDYWILINSNT | |
| | NGVVYESTNNSDFWTAVVAIEPHVNPVDRQYTIFGESKQFNVSN | |
| | DSNKWKFLEMFRSSSQNEFYNRRTLTSDTRFVGILKYGGRVWTF | |
| | HGETPRATTDSSSTANLNNISITIHSEFYIIPRSQESKCNEYIN | |
| | NGLPPIQNTRNVVPLPLSSRSIQYKRAQVNEDIIVSKTSLWKEM | |
| | QYNRDIIIRFKFGNSIVKMGGLGYKWSEISYKAANYQYNYLRDG | |
| | EQVTAHTTCSVNGVNNFSYNGGSLPTDFGISRYEVIKENSYVYV | |
| | DYWDDSKAFRNMVYVRSLAANLNSVKCTGGSYNFSIPVGAWPVM | |
| | NGGAVSLHFAGVTLSTQFTDFVSLNSLRFRFSLTVDEPPFSILR | |
| | TRTVNLYGLPAANPNNGNEYYEISGRFSLIYLVPTNDDYQTPIM | |
| | NSVTVRQDLERQLTDLREEFNSLSQEIAMAQLIDLALLPLDMFS | |
| | MFSGIKSTIDLTKSMATSVMKKFRKSKLATSISEMTNSLSDAAS | |
| | SASRNVSIRSNLSAISNWTNVSNDVSNVTNSLNDISTQTSTISK | |
| | KFRLKEMITQTEGMSFDDISAAVLKTKIDMSTQIGKNTLPDIVT | |
| | EASFEKFPKRSYRILKDDEVMEINTEGKFFAYFINTFDEVPFDV | |
| | NKFAELVTDSPVISAIIDFKTLKNLNDNYGITRTEALNLIKSNP | |
| | NMLRNFINQNNPIIRNRIEQLILQCKL | |
| Rotavirus | >Sp\|P11193\|1-230 | 83 |
| VP8 protein | MASLIYRQLLTNSYSVDLHDEIEQIGSEKTQNVTINPSPFAQTR | |
| | YAPVNWGHGEINDSTTVEPILDGPYQPTTFTPPNDYWILINSNT | |
| | NGVVYESTNNSDFWTAVVAIEPHVNPVDRQYTIFGESKQFNVSN | |
| | DSNKWKFLEMFRSSSQNEFYNRRTLTSDTRFVGILKYGGRVWTF | |
| | HGETPRATTDSSSTANLNNISITIHSEFYIIPRSQESKCNEYIN | |
| | NGLPPIQNTR | |
| Human metapneumovirus (hMPV) | >sp\|Q6WB98\|FUS_HMPVC Fusion glycoprotein F0 | 84 |
| F protein | OS = Human metapneumovirus (strain CAN97-83 | |
| | GN = F PE = 1 SV = 1 | |
| | MSWKVVIIFSLLITPQHGLKESYLEESCSTITEGYLSVLRTGWY | |
| | TNVFTLEVGDVENLTCSDGPSLIKTELDLTKSALRELKTVSADQ | |
| | LAREEQIENPRQSRFVLGAIALGVATAAAVTAGVAIAKTIRLES | |
| | EVTAIKNALKTTNEAVSTLGNGVRVLATAVRELKDFVSKNLTRA | |
| | INKNKCDIDDLKMAVSFSQFNRRFLNVVRQFSDNAGITPAISLD | |
| | LMTDAELARAVSNMPTSAGQIKLMLENRAMVRRKGFGILIGVYG | |
| | SSVIYMVQLPIFGVIDTPCWIVKAAPSCSGKKGNYACLLREDQG | |
| | WYCQNAGSTVYYPNEKDCETRGDHVFCDTAAGINVAEQSKECNI | |
| | NISTTNYPCKVSTGRHPISMVALSPLGALVACYKGVSCSIGSNR | |
| | VGIIKQLNKGCSYITNQDADTVTIDNTVYQLSKVEGEQHVIKGR | |
| | PVSSSFDPIKFPEDQFNVALDQVFENIENSQALVDQSNRILSSA | |
| | EKGNTGFIIVIILIAVLGSSMILVSIFIIIKKTKKPTGAPPELS | |
| | GVTNNGFIPHS | |
| Human metapneumovirus (hMPV) | >sp\|Q6WB94\|VGLG_HMPVC Major surface | 85 |
| G protein | glycoprotein G OS = Human metapneumovirus | |
| | (strain CAN97-83) GN = G PE = 1 SV = 1 | |
| | MEVKVENIRAIDMLKARVKNRVARSKCFKNASLILIGITTLSIA | |
| | LNIYLIINYTIQKTSSESEHHTSSPPTESNKEASTISTDNPDIN | |
| | PNSQHPTQQSTENPTLNPAASVSPSETEPASTPDTTNRLSSVDR | |
| | STAQPSESRTKTKPTVHTRNNPSTASSTQSPPRATTKAIRRATT | |
| | FRMSSTGKRPTTTSVQSDSSTTTQNHEETGSANPQASVSTMQN | |
| Human parainfluenza virus | >sp\|P12605\|FUS_PI1HC Fusion glycoprotein F0 | 86 |
| (FV) | OS = HUMAN parainfluenza 1 virus (strain C39) | |
| F protein | GN = F PE = 2 SV = 1 | |
| | MQKSEILFLIYSSLLLSSSLCQIPVDKLSNVGVIINEGKLLKIA | |
| | GSYESRYIVLSLVPSIDLEDGCGTTQIIQYKNLLNRLLIPLKDA | |
| | LDLQESLITITNDTTVTNDNPQSRFFGAVIGTIALGVATAAQIT | |
| | AGIALAEAREARKDIALIKDSIIKTHNSVELIQRGIGEQIIALK | |
| | TLQDFVNNEIRPAIGELRCETTALKLGIKLTQHYSELATAFSSN | |
| | LGTIGEKSLTLQALSSLYSANITEILSTIKKDKSDIYDIIYTEQ | |
| | VKGTVIDVDLEKYMVTLLVKIPILSEIPGVLIYRASSISYNIEG | |
| | EEWHVAIPNYIINKASSLGGADVTNCIESRLAYICPRDPTQLIP | |
| | DNQQKCILGDVSKCPVTKVINNLVPKFAFINGGVVANCIASTCT | |
| | CGTNRIPVNQDRSRGVTFLTYTNCGLIGINGIELYANKRGRDTT | |
| | WGNQIIKVGPAVSIRPVDISLNLASATNFLEESKIELMKAKAII | |
| | SAVGGWHNTESTQIIIIIIVCILIIIICGILYYLYRVRRLLVMI | |
| | NSTHNSPVNTYTLESRMRPYIGNNSN | |
| Human parainfluenza virus | >sp\|P25466\|HN_PI2HT Hemagglutinin- | 87 |
| HN protein | neuraminidase OS = Human parainfluenza 2 virus | |
| | (strain Toshiba) GN = HN PE = 2 SV = 1 | |
| | MEDYSNLSLKSIPKRTCRIIFRTATILGICTLIVLCSSILHEII | |
| | HLDVSSGLMDSDDSQQGIIQPIIESLKSLIALANQILYNVAIII | |
| | PLKIDSIETVIFSALKDMHTGSMSNTNCTPGNLLLHDAAYINGI | |
| | NKFLVLKSYNGTPKYGPLLNIPSFIPSATSPNGCTRIPSFSLIK | |
| | THWCYTHNVMLGDCLDFTTSNQYLAMGIIQQSAAAFPIFRTMKT | |
| | IYLSDGINRKSCSVTAIPGGCVLYCYVATRSEKEDYATTDLAEL | |
| | RLAFYYYNDTFIERVISLPNTTGQWATINPAVGSGIYHLGFILF | |
| | PVYGGLISGTPSYNKQSSRYFIPKHPNITCAGNSSEQAAAARSS | |
| | YVIRYHSNRLIQSAVLICPLSDMHTARCNLVMFNNSQVMMGAEG | |
| | RLYVIDNNLYYYQRSSSWWSASLFYRINTDFSKGIPPIIEAQWV | |
| | PSYQVPRPGVMPCNATSFCPANCITGVYADVWPLNDPEPTSQNA | |
| | LNPNYRFAGAFLRNESNRTNPTFYTASASALLNTTGFNNTNHKA | |
| | AYTSSTCFKNTGTQKIYCLIIIEMGSSLLGEFQIIPFLRELIP | |
| Malaria | >sp\|P13829\|OS25_PLAFO 25 kDa ookinete | 88 |
| Pfs25 surface antigen | surface antigen OS = Plasmodium falciparum | |
| | (isolate NF54) PE = 1 SV = 1 | |
| | MNKLYSLFLFLFIQLSIKYNNAKVTVDTVCKRGFLIQMSGHLEC | |
| | KCENDLVLVNEETCEEKVLKCDEKTVNKPCGDFSKCIKIDGNPV | |
| | SYACKCNLGYDMVNNVCIPNECKNVTCGNGKCILDTSNPVKTGV | |
| | CSCNIGKVPNVQDQNKCSKDGETKCSLKCLKENETCKAVDGIYK | |
| | CDCKDGFIIDNESSICTAFSAYNILNLSIMFILFSVCFFIM | |
| serogroup B Neisseria | >tr\|Q6QCC2\|Q6QCC2_NEIME Factor H-binding | 98 |
| meningitidis (MenB) | protein OS = Neisseria meningitidis OX = 487 | |
| fHbp | GN = gna1870 PE = 1 SV = 1 | |
| | MNPTAFCCLSLTTALILTACSSGGGGVAADIGAGLADALTAPLD | |
| | HKDKGLQSLTLDQSVRKNEKLKLAAQGAEKTYGNGDSLNTGKLK | |
| | NDKVSPFDFIRQIEVDGQLITLESGEFQVYKQSHSALTAFQTEQ | |
| | IQDSEHSGKMVAKRQFRIGDIAGEHTSFDKLPEGGRATYRGTAF | |
| | GSDDAGGKLTYTIDFAAKQGNGKIEHLKSPELNVDLAAADIKPD | |
| | GKRHAVISGSVLYNQAEKGSYSLGIFGGKAQEVAGSAEVKTVNG | |
| | IRHIGLAAKQ | |
| serogroup B Neisseria | >tr\|X5F9B9\|X5F9B9_NEIME Quinolinate synthase | 99 |
| meningitidis (MenB) | A OS = Neisseria meningitdis OS = 487 GN = nadA2 | |
| NadA | PE = 3 SV = 1 | |
| | MQTAARRSFDYDMPLIQTPTSACQIRQAWAKVADTPDRETAGRL | |
| | KDEIKALLKETNAVLVAHYYVDPLIQDLALETGGCVGDSLEMAR | |
| | FGAEHEAGTLVVAGVRFMGESAKILCPEKTVLMPDLEAECSLDL | |
| | GCPEEAFSAFCDQHPDRTVVVYANTSAAVKARADWVVTSSVALE | |
| | IVSYLKSRGEKLIWGPDRHLGDYIRRETGADMLLWQGSCIVHNE | |
| | FKGQELAALKAEHPDAVVLVHPESPQSVIELGDVVGSTSKLLKA | |
| | AVSRPEKKFIVATDLGILHEMQKQAPDKQFIAAPTAGNGGSCKS | |
| | CAFCPWMAMNSLGGIKYALTSGHNEILLDRKLGEAAKLPLQRML | |
| | DFAAGLKRGDVFNGMGPA | |
| serogroup B Neisseria | >tr\|Q9JPH1\|Q9JPH1_NEIME Gna2132 OS = Neisseria | 100 |
| meningitidis (MenB) | meningitidis OX = 487 GN = gna2132 PE = 4 SV = 1 | |
| NHBA | MFKRSVIAMACIFALSACGGGGGGSPDVKSADTLSKPAAPVVSE | |
| | KETEAKEDAPQAGSQGQGAPSAQGGQDMAAVSEENTGNGGAAAT | |
| | DKPKNEDEGAQNDMPQNAADTDSLTPNHTPASNMPAGNMENQAP | |
| | DAGESEQPANQPDMANTADGMQGDDPSAGGENAGNTAAQGTNQA | |
| | ENNQTAGSQNPASSTNPSATNSGGDFGRTNVGNSVVIDGPSQNI | |
| | TLTHCKGDSCSGNNFLDEEVQLKSEFEKLSDADKISNYKKDGKN | |
| | DGKNDKFVGLVADSVQMKGINQYIIFYKPKPTSFARFRRSARSR | |
| | RSLPAEMPLIPVNQADTLIVDGEAYSLTGHSGNIFAREGNYRYL | |
| | TYGAEKLPGGSYALRVQGEPSKGEMLAGTAVYNGEVLHFHTENG | |
| | RPSPSRGRFAAKVDFGSKSVDGIIDSGDGLHMGTQKFKAAIDGN | |
| | GFKGTWTENGGGDVSGKFYGPAGEEVAGKYSYRPTDAEKGGFGV | |
| | FAGKKEQD | |

### 4. Assembly Domains and Linkers

In an embodiment, the nanostructure comprises a trimeric assembly. The trimeric assembly comprises a protein-protein interface that induces three copies of the first polypeptides to self-associate to form trimeric building blocks. Each copy of the first polypeptides further comprises a surface-exposed interface that interacts with a complementary surface-exposed interface on a second assembly domain. As described in King et al. (Nature 510, 103-108, 2014), Bale et al. (Science 353, 389-394, 2016), and patent publications WO2014124301 A1 and US20160122392 A1, the complementary protein-protein interface between the trimeric assembly domain and second assembly domain drives the assembly of multiple copies of the trimeric assembly domain and second assembly domain to a target nanostructure. In some embodiments, each copy of the trimeric assembly domains of the nanostructure bears an antigenic proteins, or antigenic fragment thereof, as a genetic fusion: these nanostructures display the proteins at full valency. In other embodiments, the nanostructures of the invention comprise one or more copies of trimeric assembly domains bearing antigens proteins, or antigenic fragments thereof as genetic fusions as well as one or more trimeric assembly domains that do not bear antigenic proteins as genetic fusions; these nanostructures display the F proteins at partial valency. The trimeric assembly domain can be any polypeptide sequence that forms a trimer and interacts with a second assembly domain to drive assembly to a target nanostructure. In some embodiments. the nanostructure comprises first and second polypeptides selected from those disclosed in US 20130274441 A1, US 2015/0356240 A1, US 2016/0122392 A1, WO 2018/187325 A1, each of which is incorporated by reference herein in its entirety.

In the nanostructures of the present disclosure, the antigenic protein and the core of the nanostructure may be genetically fused such that they are both present in a single polypeptide. Preferably, the linkage between the protein and the core of the nanostructure allows the protein, or antigenic fragment thereof, to be displayed on the exterior of the nanostructure. As such, the point of connection to the core of the nanostructure should be on the exterior of the core of the nanostructure formed. As will be understood by those of skill in the art, a wide variety of polypeptide sequences can be used to link the proteins, or antigenic fragments thereof and the core of the nanostructure. These polypeptide sequences are referred to as linkers. Any suitable linker can be used; there is no amino acid sequence requirement to serve as an appropriate linker. There is no requirement that the linker impose a rigid relative orientation of the protein or antigenic fragment thereof to the core of the nanostructure beyond enabling the protein or antigenic fragment thereof to be displayed on the exterior of the nanostructure. In some embodiments, the linker includes additional trimerization domains (e.g., the foldon domain of T4 fibritin) that assist in stabilizing the trimeric form of the F protein.

>T4 fibritin foldon domain (optional in the linker
region)
(SEQ ID NO: 89)
GYIPEAPRDGQAYVRKDGEWVLLSTFL

In some embodiments, the linker may comprise a Gly-Ser linker (i.e. a linker consisting of glycine and serine residues) of any suitable length. In some embodiments, the Gly-Ser linker may be 3, 4, 5, 6, 7, 8, 9, 10, 11, 12, 13, 14, 15, 16, 17, 18, 19, 20, or more amino acids in length. In some embodiments, the Gly-Ser linker may comprise or consist of the amino acid sequence of GSGGSGSGSGGSGSG, GGSGGSGS or GSGGSGSG.

### 5. Assembly of Nanostructures

In some embodiments, one or more purified samples of pluralities of the polypeptides for use in forming a nanostructure are mixed in an approximately equimolar molar ratio in aqueous conditions. The polypeptides interact with one another to drive assembly of the target nanostructure. Successful assembly of the target nanostructure can be confirmed by analyzing the in vitro assembly reaction by common biochemical or biophysical methods used to assess the physical size of proteins or protein assemblies, including but not limited to size exclusion chromatography, native (non-denaturing) gel electrophoresis, dynamic light scattering, multi-angle light scattering, analytical ultracentrifugation, negative stain electron microscopy, cryo-electron microscopy, or X-ray crystallography. If necessary, the assembled nanostructure can be purified from other species or molecules present in the in vitro assembly reaction using preparative techniques commonly used to isolate proteins by their physical size, including but not limited to size exclusion chromatography, preparative ultracentrifugation, tangential flow filtration, or preparative gel electrophoresis. The presence of the antigenic protein in the nanostructure can be assessed by techniques commonly used to determine the identity of protein molecules in aqueous solutions, including but not limited to SDS-PAGE, mass spectrometry, protein sequencing, or amino acid analysis. The accessibility of the protein on the exterior of the particle, as well as its conformation or antigenicity, can be assessed by techniques commonly used to detect the presence and conformation of an antigen, including but not limited to binding by monoclonal antibodies, conformation-specific monoclonal antibodies, or anti-sera specific to the antigen.

In other embodiments, the nanostructures of the invention comprise two or more distinct first polypeptides bearing different antigenic proteins as genetic fusions: these nanostructures co-display multiple different proteins on the same nanostructure. These multi-antigen nanostructures are produced by performing in vitro assembly with mixtures of first polypeptides in which each first polypeptide bears one of two or more distinct proteins as a genetic fusion. The fraction of each first polypeptide in the mixture determines the average valency of each antigenic protein in the resulting nanostructures. The presence and average valency of each protein-bearing first polypeptides in a given sample can be assessed by quantitative analysis using the techniques described above for evaluating the presence of antigenic proteins in full-valency nanostructures.

In various embodiments, the nanostructures are between about 20 nanometers (nm) to about 40 nm in diameter, with interior lumens between about 15 nm to about 32 nm across and pore sizes in the protein shells between about 1 nm to about 14 nm in their longest dimensions.

In one embodiment, the nanostructure has icosahedral symmetry. In this embodiment, the nanostructure may comprise 60 copies of a first polypeptide and 60 copies of a second polypeptide. In one such embodiment, the number of identical first polypeptides in each first assembly is different than the number of identical second polypeptides in each second assembly. For example, in one embodiment, the nanostructure comprises twelve first assemblies and twenty second assemblies: in this embodiment, each first assembly may, for example, comprise five copies of the identical first polypeptide, and each second assembly may, for example, comprise three copies of the identical second polypeptide. In another embodiment, the nanostructure comprises twelve first assemblies and thirty second assemblies; in this embodiment, each first assembly may, for example, comprise five copies of the identical first polypeptide, and each second assembly may, for example, comprise two copies of the identical second polypeptide. In a further embodiment, the nanostructure comprises twenty first assemblies and thirty second assemblies; in this embodiment, each first assembly may, for example, comprise three copies of the identical first polypeptide, and each second assembly may, for example, comprise two copies of the identical second polypeptide. All of these embodiments are capable of forming synthetic nanomaterials with regular icosahedral symmetry.

In various further embodiments, oligomeric states of the first and second polypeptides are as follows:
I53-34A: trimer+I53-34B: pentamer;
I53-40A: pentamer+I53-40B: trimer:
I53-47A: trimer+I53-47B: pentamer;
I53-50A: trimer+I53-50B: pentamer;
I53-51A: trimer+I53-51B: pentamer;
I32-06A: dimer+I32-06B: trimer;
I32-19A: trimer+I32-19B: dimer;
I32-28A: trimer+I32-28B: dimer;
I52-03A: pentamer+I52-03B: dimer:
I52-32A: dimer+I52-32B: pentamer; and
I52-33A: pentamer+I52-33B: dimer

### 6. Nucleic Acids

In another aspect, the present disclosure provides isolated nucleic acids encoding a fusion protein of the present disclosure. The isolated nucleic acid sequence may comprise RNA or DNA. As used herein, “isolated nucleic acids” are those that have been removed from their normal surrounding nucleic acid sequences in the genome or in cDNA sequences. Such isolated nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the proteins of the disclosure.

In a further aspect, the present disclosure provides recombinant expression vectors comprising the isolated nucleic acid of any embodiment or combination of embodiments of the disclosure operatively linked a suitable control sequence. “Recombinant expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type known in the art. including but not limited to plasmid and viral-based expression vectors. The control sequence used to drive expression of the disclosed nucleic acid sequences in a mammalian system may be constitutive (driven by any of a variety of promoters, including but not limited to, CMV. SV40, RSV, actin, EF) or inducible (driven by any of a number of inducible promoters including, but not limited to, tetracycline, ecdysone, steroid responsive). The construction of expression vectors for use in transfecting prokaryotic cells is also well known in the art, and thus can be accomplished via standard techniques. (See, for example, Sambrook, Fritsch, and Maniatis, in: Molecular Cloning, A Laboratory Manual. Cold Spring Harbor Laboratory Press, 1989; Gene Transfer and Expression Protocols, pp. 109-128, ed. E. J. Murray, The Humana Press Inc., Clifton, N.J.), and the Ambion 1998 Catalog (Ambion, Austin, TX). The expression vector must be replicable in the host organisms either as an episome or by integration into host chromosomal DNA. In a preferred embodiment, the expression vector comprises a plasmid. However, the disclosure is intended to include other expression vectors that serve equivalent functions, such as viral vectors.

In another aspect, the present disclosure provides host cells that have been transfected with the recombinant expression vectors disclosed herein, wherein the host cells can be either prokaryotic or eukaryotic. The cells can be transiently or stably transfected. Such transfection of expression vectors into prokaryotic and eukaryotic cells can be accomplished via any technique known in the art, including but not limited to standard bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press; Culture of Animal Cells: A Manual of Basic Technique, 2nd Ed. (R. I. Freshney, 1987. Liss, Inc. New York, NY). A method of producing a polypeptide according to the disclosure is an additional part of the disclosure. The method comprises the steps of (a) culturing a host according to this aspect of the disclosure under conditions conducive to the expression of the polypeptide, and (b) optionally, recovering the expressed polypeptide.

### 7. Vaccines and Administration

The disclosure also provides vaccines comprising the nanostructures described herein. Such compositions can be used to raise antibodies in a mammal (e.g. a human). The vaccines compositions of the disclosure typically include a pharmaceutically acceptable carrier, and a thorough discussion of such carriers is available in Remington: The Science and Practice of Pharmacy.

The pH of the composition is usually between about 4.5 to about 11, such as between about 5 to about 11. between about 5.5 to about 11, between about 6 to about 11, between about 5 to about 10.5. between about 5.5 to about 10.5, between about 6 to about 10.5, between about 5 to about 10, between about 5.5 to about 10, between about 6 to about 10, between about 5 to about 9.5, between about 5.5 to about 9.5, between about 6 to about 9.5. between about 5 to about 9, between about 5.5 to about 9, between about 6 to about 9, between about 5 to about 8.5. between about 5.5 to about 8.5. between about 6 to about 8.5. between about 5 to about 8, between about 5.5 to about 8, between about 6 to about 8, about 4.5, about 5, about 6.5, about 6, about 6.5, about 7, about 7.5, about 8, about 8.5, about 9, about 9.5, about 10, about 10.5, about 11. etc. Stable pH may be maintained by the use of a buffer e.g. a Tris buffer, a citrate buffer, phosphate buffer, or a histidine buffer. Thus a composition will generally include a buffer.

A composition may be sterile and/or pyrogen free. Compositions may be isotonic with respect to humans.

A vaccine composition comprises an immunologically effective amount of its antigen(s). An “immunologically effective amount” is an amount which, when administered to a subject, is effective for eliciting an antibody response against the antigen. This amount can vary depending upon the health and physical condition of the individual to be treated, their age, the capacity of the individual's immune system to synthesize antibodies, the degree of protection desired, the formulation of the vaccine, the treating doctor's assessment of the medical situation, and other relevant factors. It is expected that the amount will fall in a relatively broad range that can be determined through routine trials. The antigen content of compositions of the disclosure will generally be expressed in terms of the mass of protein per dose. A dose of 10-500 μg (e.g. 50 μg) per antigen can be useful.

Vaccine compositions may include an immunological adjuvant. Exemplary adjuvants include the following: 1. mineral-containing compositions; 2. oil emulsions: 3. saponin formulations: 4. virosomes and virus-like particles: 5. bacterial or microbial derivatives: 6. bioadhesives and mucoadhesives; 7. liposomes; 8. polyoxyethylene ether and polyoxyethylene ester formulations: 9. polyphosphazene (pcpp); 10. muramyl peptides; 11. imidazoquinolone compounds: 12. thiosemicarbazone compounds: 13. tryptanthrin compounds: 14. human immunomodulators: 15. lipopeptides: 16. benzonaphthyridines: 17. microparticles: 18. immunostimulatory polynucleotide (such as rna or dna; e.g., cpg-containing oligonucleotides).

For example, the composition may include an aluminum salt adjuvant, an oil in water emulsion (e.g. an oil-in-water emulsion comprising squalene, such as MF59 or AS03). a TLR7 agonist (such as imidazoquinoline or imiquimod), or a combination thereof. Suitable aluminum salts include hydroxides (e.g., oxyhydroxides), phosphates (e.g. hydroxyphosphates, orthophosphates), (e.g., see chapters 8 & 9 of Vaccine Design . . . (1995) eds. Powell & Newman. ISBN: 030644867X. Plenum), or mixtures thereof. The salts can take any suitable form (e.g. gel, crystalline, amorphous, etc.), with adsorption of antigen to the salt being an example. The concentration of Al⁺⁺⁺ in a composition for administration to a patient may be less than 5mg/ml e.g. <4 mg/ml, <3 mg/ml, <2 mg/ml, <1 mg/ml, etc. A preferred range is between 0.3 and 1 mg/ml. A maximum of 0.85 mg/dose is preferred. Aluminum hydroxide and aluminium phosphate adjuvants are suitable for use with the disclosure.

Exemplary adjuvants include, but are not limited to. Adju-Phos, Adjumerlm, albumin-heparin microparticles, Algal Glucan, Algammulin, Alum, Antigen Formulation, AS-2 adjuvant, autologous dendritic cells, autologous PBMC, Avridine™, B7-2, BAK, BAY R1005, Bupivacaine. Bupivacaine-HCl, BWZL, Calcitriol, Calcium Phosphate Gel, CCR5 peptides, CFA, Cholera holotoxin (CT) and Cholera toxin B subunit (CTB), Cholera toxin Al-subunit-Protein A D-fragment fusion protein, CpG. CRL1005, Cytokine-containing Liposomes, D-Murapalmitine, DDA, DHEA, Diphtheria toxoid, DL-PGL, DMPC, DMPG, DOC/Alum Complex, Fowlpox, Freund's Complete Adjuvant, Gamma Inulin, Gerbu Adjuvant, GM-CSF, GMDP, hGM-CSF, hIL-12 (N222L), hTNF-alpha, IFA, IFN-gamma in pcDNA3, IL-12 DNA, IL-12 plasmid, IL-12/GMCSF plasmid (Sykes), IL-2 in pcDNA3, IL-2/Ig plasmid, IL-2/Ig protein, IL-4, IL-4 in pcDNA3, Imiquimod, Imm Ther™, Immunoliposomes Containing Antibodies to Costimulatory Molecules, Interferon-gamma, Interleukin-1 beta, Interleukin-12, Interleukin-2, Interleukin-7, ISCOM(s)™, Iscoprep 7.0.3™, Keyhole Limpet Hemocyanin. Lipid-based Adjuvant. Liposomes. Loxoribine, LT(R192G), LT-OA or LT Oral Adjuvant, LT-R192G, LTK63, LTK72, MF59, MONTANIDE ISA 51, MONTANIDE ISA 720, MPL.TM., MPL-SE, MTP-PE, MTP-PE Liposomes. Murametide, Murapalmitine, NAGO, nCT native Cholera Toxin, Non-Ionic Surfactant Vesicles, non-toxic mutant E1 12K of Cholera Toxin mCT-E112K, p-Hydroxybenzoique acid methyl ester, pCIL-10, pCIL12, pCMVmCATI, pCMVN, Peptomer-NP, Pleuran, PLG, PLGA, PGA, and PLA. Pluronic L121, PMMA. PODDS™, Poly rA: Poly rU, Polysorbate 80, Protein Cochleates, QS-21. Quadri A saponin, Quil-A, Rehydragel HPA, Rehydragel LV, RIBI, Ribilike adjuvant system (MPL, TMD, CWS), S-28463, SAF-1, Sclavo peptide, Sendai Proteoliposomes, Sendai-containing Lipid Matrices, Span 85, Specol, Squalane 1. Squalene 2, Stearyl Tyrosine. Tetanus toxoid (TT), Theramide™, Threonyl muramyl dipeptide (TMDP), Ty Particles, and Walter Reed Liposomes. Selection of an adjuvant depends on the subject to be treated. Preferably, a pharmaceutically acceptable adjuvant is used.

One suitable immunological adjuvant comprises a compound of Formula (I) as defined in WO2011/027222. or a pharmaceutically acceptable salt thereof, adsorbed to an aluminum salt. Many further adjuvants can be used, including any of those disclosed in Powell & Newman (1995).

Compositions may include an antimicrobial, particularly when packaged in multiple dose format. Antimicrobials such as thiomersal and 2-phenoxyethanol are commonly found in vaccines, but sometimes it may be desirable to use either a mercury-free preservative or no preservative at all.

Compositions may comprise detergent e.g., a polysorbate, such as polysorbate 80. Detergents are generally present at low levels e.g. <0.01%.

Compositions may include sodium salts (e.g., sodium chloride) to give tonicity. A concentration of 10±2 mg/ml NaCl is typical e.g., about 9 mg/ml.

In some embodiments, the buffer in the vaccine composition is a Tris buffer, a histidine buffer, a phosphate buffer, a citrate buffer or an acetate buffer. The composition may also include a lyoprotectant, e.g., sucrose, sorbitol or trehalose. In certain embodiments, the composition includes a preservative e.g., benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the composition includes a bulking agent, like glycine. In yet other embodiments, the composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the composition additionally includes a stabilizer, e.g., a molecule which substantially prevents or reduces chemical and/or physical instability of the nanostructure, in lyophilized or liquid form. Exemplary stabilizers include sucrose, sorbitol, glycine, inositol, sodium chloride, methionine, arginine, and arginine hydrochloride.

In another aspect, the disclosure provides a method of inducing an immune response against an infectious agent, comprising administering to a subject in need thereof an immunologically effective amount of the immunogenic composition described herein, which comprises the nanostructure as described herein.

In certain embodiments, the immune response comprises the production of neutralizing antibodies against an infectious agent. In certain embodiments, the neutralizing antibodies are complement-independent.

The immune response can comprise a humoral immune response, a cell-mediated immune response, or both. In some embodiments an immune response is induced against each delivered antigenic protein. A cell-mediated immune response can comprise a Helper T-cell (Th) response, a CD8+ cytotoxic T-cell (CTL) response, or both. In some embodiments the immune response comprises a humoral immune response, and the antibodies are neutralizing antibodies. Neutralizing antibodies block viral infection of cells. Viruses infect epithelial cells and also fibroblast cells. In some embodiments the immune response reduces or prevents infection of both cell types. Neutralizing antibody responses can be complement-dependent or complement-independent. In some embodiments the neutralizing antibody response is complement-independent. In some embodiments the neutralizing antibody response is cross-neutralizing: i.e., an antibody generated against an administered composition neutralizes a virus of a strain other than the strain used in the composition.

A useful measure of antibody potency in the art is “50% neutralization titer.” To determine 50% neutralizing titer, serum from immunized animals is diluted to assess how dilute serum can be yet retain the ability to block entry of 50% of viruses into cells. For example, a titer of 700 means that serum retained the ability to neutralize 50% of virus after being diluted 700-fold. Thus, higher titers indicate more potent neutralizing antibody responses. In some embodiments, this titer is in a range having a lower limit of about 200, about 400, about 600. about 800, about 1000. about 1500, about 2000, about 2500, about 3000, about 3500, about 4000, about 4500, about 5000, about 5500, about 6000, about 6500, or about 7000. The 50% neutralization titer range can have an upper limit of about 400, about 600, about 800, about 1000, about 1500, about 2000, about 2500. about 3000, about 3500. about 4000, about 4500, about 5000, about 5500, about 6000, about 6500, about 7000, about 8000, about 9000, about 10000, about 11000, about 12000, about 13000, about 14000, about 15000. about 16000, about 17000, about 18000. about 19000, about 20000, about 21000. about 22000, about 23000, about 24000, about 25000. about 26000, about 27000, about 28000, about 29000, or about 30000. For example, the 50% neutralization titer can be about 3000 to about 25000. “About” means plus or minus 10% of the recited value.

Compositions of the disclosure will generally be administered directly to a subject. Direct delivery may be accomplished by parenteral injection (e.g., subcutaneously, intraperitoneally, intravenously, intramuscularly, or to the interstitial space of a tissue), or by any other suitable route. For example, intramuscular administration may be used e.g., to the thigh or the upper arm. Injection may be via a needle (e.g., a hypodermic needle), but needle-free injection may alternatively be used. A typical intramuscular dosage volume is 0.5 ml.

Dosage can be by a single dose schedule or a multiple dose schedule. Multiple doses may be used in a primary immunization schedule and/or in a booster immunization schedule. In a multiple dose schedule the various doses may be given by the same or different routes, e.g., a parenteral prime and mucosal boost, a mucosal prime and parenteral boost, etc. Multiple doses will typically be administered at least 1 week apart (e.g., about 2 weeks, about 3 weeks, about 4 weeks, about 6 weeks, about 8 weeks, about 10 weeks, about 12 weeks, about 16 weeks, etc.).

The subject may be an animal, preferably a vertebrate, more preferably a mammal. Exemplary subject includes, e.g., a human, a cow, a pig, a chicken, a cat or a dog, as the infectious agents covered herein may be problematic across a wide range of species. Where the vaccine is for prophylactic use, the human is preferably a child (e.g., a toddler or infant), a teenager, or an adult; where the vaccine is for therapeutic use, the human is preferably a teenager or an adult. A vaccine intended for children may also be administered to adults, e.g., to assess safety, dosage, immunogenicity, etc.

Vaccines of the disclosure may be prophylactic (i.e, to prevent disease) or therapeutic (i.e, to reduce or eliminate the symptoms of a disease). The term prophylactic may be considered as reducing the severity of or preventing the onset of a particular condition. For the avoidance of doubt, the term prophylactic vaccine may also refer to vaccines that ameliorate the effects of a future infection, for example by reducing the severity or duration of such an infection.

Isolated and/or purified nanostructures described herein can be administered alone or as either prime or boost in mixed-modality regimes, such as a RNA prime followed by a protein boost. Benefits of the RNA-prime/protein-boost strategy, as compared to a protein-prime/protein-boost strategy, include, for example, increased antibody titers, a more balanced lgG1:IgG2a subtype profile, induction of TH1-type CD4+ T cell-mediated immune response that was similar to that of viral particles, and reduced production of non-neutralizing antibodies. The RNA prime can increase the immunogenicity of compositions regardless of whether they contain or do not contain an adjuvant.

In the RNA-prime/protein boost-strategy, the RNA and the protein are directed to the same target antigen. Examples of suitable modes of delivering RNAs include virus-like replicon particles (VRPs), alphavirus RNA, replicons encapsulated in lipid nanoparticles (LNPs) or formulated RNAs, such as replicons formulated with cationic nanoemulsions (CNEs). Suitable cationic oil-in-water nanoemulsions are disclosed in WO2012/006380 e.g. comprising an oil core (e.g. comprising squalene) and a cationic lipid (e.g. DOTAP, DMTAP, DSTAP, DC-cholesterol, etc.).

In some embodiments, the RNA molecule is encapsulated in, bound to or adsorbed on a cationic lipid, a liposome, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, a polycationic peptide, a cationic nanoemulsion, or combinations thereof.

Also provided herein are kits for administration of nucleic acid (e.g., RNA), purified proteins, and purified nanostructures described herein, and instructions for use. The disclosure also provides a delivery device pre-filled with a composition or a vaccine disclosed herein.

The pharmaceutical compositions described herein can be administered in combination with one or more additional therapeutic agents. The additional therapeutic agents may include, but are not limited to antibiotics or antibacterial agents, antiemetic agents, antifungal agents, anti-inflammatory agents, antiviral agents, immunomodulatory agents, cytokines, antidepressants, hormones, alkylating agents, antimetabolites, antitumour antibiotics, antimitotic agents, topoisomerase inhibitors, cytostatic agents, anti-invasion agents, antiangiogenic agents, inhibitors of growth factor function inhibitors of viral replication, viral enzyme inhibitors, anticancer agents, α-interferons, β-interferon, ribavirin, hormones, and other toll-like receptor modulators, immunoglobulins (Igs), and antibodies modulating Ig function (such as anti-IgE (omalizumab)).

In certain embodiments, the compositions disclosed herein may be used as a medicament, e.g., for use in inducing or enhancing an immune response in a subject in need thereof, such as a mammal.

In certain embodiments, the compositions disclosed herein may be used in the manufacture of a medicament for inducing or enhancing an immune response in a subject in need thereof, such as a mammal.

One way of checking efficacy of therapeutic treatment involves monitoring infection by an infectious agent after administration of the compositions or vaccines disclosed herein. One way of checking efficacy of prophylactic treatment involves monitoring immune responses, systemically (such as monitoring the level of IgG1 and IgG2a production) and/or mucosally (such as monitoring the level of IgA production), against the antigen. Typically, antigen-specific serum antibody responses are determined post-immunization but pre-challenge whereas antigen-specific mucosal antibody responses are determined post-immunization and post-challenge.

### 8. Terminology

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2^{nd} Ed (R I. Freshney, 1987, Liss, Inc. New York, NY), Gene Transfer and Expression Protocols, pp. 109-128, ed. E. J. Murray, The Humana Press Inc., Clifton, NJ.), and the Ambion 1998 Catalog (Ambion, Austin, TX).

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%. It should be understood that the terms “a” and “an” as used herein refer to “one or more” of the enumerated components unless otherwise indicated. The use of the alternative (e.g., “or”) should be understood to mean either one, both, or any combination thereof of the alternatives. The term “and/or” should be understood to mean either one, or both of the alternatives. As used herein, the terms “include” and “comprise” are used synonymously.

The section headings used herein are for organizational purposes only and are not to be construed as limiting the subject matter described.

Unless specifically defined otherwise, the following terms and phrases, which are common to the various embodiments disclosed herein, are defined as follows:

As used herein, the term protein refers to a protein or a glycoprotein.

As used herein, the term immunogenic refers to the ability of a specific protein, or a specific region thereof, to elicit an immune response to the specific protein, or to proteins comprising an amino acid sequence having a high degree of identity with the specific protein. According to the present disclosure, two proteins having a high degree of identity have amino acid sequences at least 80% identical, at least 85% identical, at least 87% identical, at least 90% identical, at least 92% identical, at least 94% identical, at least 96% identical, at least 98% identical or at least 99% identical.

As used herein, an immune response to a vaccine, or nanostructure, of the present disclosure is the development in a subject of a humoral and/or a cellular immune response to an antigenic protein present in the vaccine. For purposes of the present disclosure, a “humoral immune response” refers to an immune response mediated by antibody molecules, including secretory (IgA) or IgG molecules, while a “cellular immune response” is one mediated by T-lymphocytes and/or other white blood cells. One important aspect of cellular immunity involves an antigen-specific response by cytolytic T-cells (“CTLs”). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A cellular immune response also refers to the production of cytokines, chemokines and other such molecules produced by activated T-cells and/or other white blood cells, including those derived from CD4+ and CD8+ T-cells.

Thus, an immunological response may be one that stimulates CTLs, and/or the production or activation of helper T-cells. The production of chemokines and/or cytokines may also be stimulated. The vaccine may also elicit an antibody-mediated immune response. Hence, an immunological response may include one or more of the following effects: the production of antibodies (e.g., IgA or IgG) by B-cells; and/or the activation of suppressor. cytotoxic, or helper T-cells and/or T-cells directed specifically to a hemagglutinin protein present in the vaccine. These responses may serve to neutralize infectivity, and/or mediate antibody-complement, or antibody dependent cell cytotoxicity (ADCC) to provide protection to an immunized individual. Such responses can be determined using standard immunoassays and neutralization assays, well known in the art.

As used herein the term “antibody” includes intact molecules as well as functional fragments thereof, such as Fab, F(ab′)2, Fv, scFv, dsFv, or single domain molecules such as VH and VL that are capable of specifically binding to an epitope of an antigen. The term “antibody” encompasses B-cell receptors. The term “antibody” further encompasses camelid antibodies.

As used herein in describing viruses, neutralizing antibodies are antibodies that prevent virus from completing one round of replication. As defined herein, one round of replication refers the life cycle of the virus, starting with attachment of the virus to a host cell and ending with budding of newly formed virus from the host cell. This life cycle includes. but is not limited to, the steps of attaching to a cell, entering a cell, cleavage and rearrangement of viral proteins, fusion of the viral membrane with the endosomal membrane, release of viral ribonucleoproteins into the cytoplasm, formation of new viral particles and budding of viral particles from the host cell membrane.

As used herein, broadly neutralizing antibodies are antibodies that neutralize more than one type, subtype and/or strain of bacteria, virus, or parasite. For example, broadly neutralizing antibodies elicited against an influenza HA protein from a Type A influenza virus may neutralize a Type B or Type C virus. As a further example, broadly neutralizing antibodies elicited against an influenza HA protein from Group I influenza virus may neutralize a Group 2 virus. As an additional example, broadly neutralizing antibodies elicited against an HA protein from one sub-type or strain of virus, may neutralize another sub-type or strain of virus. For example, broadly neutralizing antibodies elicited against an HA protein from an HI influenza virus may neutralize viruses from one or more sub-types selected from the group consisting of H2, H3, H4, H5, H6, H7, H8, H8, H10, H11, H12, H13, H14, H15 or H16.

With regard to antigens, it is understood by those skilled in the art that antigen proteins from different strains may have different lengths due to mutations (insertions, deletions) in the protein. Thus, reference to a corresponding region refers to a region of another proteins that is identical, or nearly so (e.g., at least 95%, identical, at least 98% identical or at least 99% identical), in sequence, structure and/or function to the region being compared. For example, with regard to an epitope of a protein, the corresponding region in a corresponding protein from a different strain of the organism may not have the same residue numbers, but will have a similar or nearly identical sequence and will perform the same function. To better clarify sequences comparisons between strains, numbering systems are used by those in the field, which relate amino acid positions to a reference sequence. Thus, corresponding amino acid residues in antigen proteins from different strains may not have the same residue number with respect to their distance from the N-terminal amino acid of the protein. The use of such numbering systems is understood by those skilled in the art.

According to the present disclosure, a trimerization domain is a series of amino acids that when joined (also referred to as fused) to a protein or peptide, allow the fusion protein to interact with other fusion proteins containing the trimerization domain, such that a trimeric structure is formed. Any known trimerization domain can be used in the present disclosure. Examples of trimerization domains include, but are not limited to, the HIV-1 gp41 trimerization domain, the SIV gp41 trimerization domain, the Ebola virus gp-2 trimerization domain, the HTLV-1 gp-21 trimerization domain, the T4 fibritin trimerization domain (i.e., foldon), the yeast heat shock transcription factor trimerization domain, and the human collagen trimerization domain.

As used herein, a variant refers to a protein, or nucleic acid molecule, the sequence of which is similar, but not identical to, a reference sequence, wherein the activity of the variant protein (or the protein encoded by the variant nucleic acid molecule) is not significantly altered. These variations in sequence can be naturally occurring variations or they can be engineered through the use of genetic engineering technique known to those skilled in the art. Examples of such techniques are found in Sambrook J, Fritsch E F, Maniatis T et al., in Molecular Cloning-A Laboratory Manual, 2nd Edition, Cold Spring Harbor Laboratory Press, 1989, pp. 9.31-9.57), or in Current Protocols in Molecular Biology, John Wiley & Sons, N.Y. (1989). 6.3.1-6.3.6, both of which are incorporated herein by reference in their entirety.

With regard to variants, any type of alteration in the amino acid, or nucleic acid. sequence is permissible so long as the resulting variant protein retains the ability to elicit neutralizing antibodies against an influenza virus. Examples of such variations include, but are not limited to, deletions, insertions, substitutions and combinations thereof. For example, with regard to proteins, it is well understood by those skilled in the art that one or more (e.g., 2, 3, 4, 5, 6, 7, 8, 9 or 10), amino acids can often be removed from the amino and/or carboxy terminal ends of a protein without significantly affecting the activity of that protein. Similarly, one or more (e.g., 2, 3. 4, 5, 6, 7, 8, 9 or 10) amino acids can often be inserted into a protein without significantly affecting the activity of the protein.

As noted, variant proteins of the present disclosure can contain amino acid substitutions relative to the nanostructure antigen proteins disclosed herein. Any amino acid substitution is permissible so long as the activity of the protein is not significantly affected. In this regard, it is appreciated in the art that amino acids can be classified into groups based on their physical properties. Examples of such groups include, but are not limited to, charged amino acids, uncharged amino acids, polar uncharged amino acids, and hydrophobic amino acids. Preferred variants that contain substitutions are those in which an amino acid is substituted with an amino acid from the same group. Such substitutions are referred to as conservative substitutions.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), praline (Pro; P), serine (Ser; S), threonine (Thr; T), tryptophan (Trp; W), tyrosine (Tyr; Y), and valine (Val; V). As used herein, “about” means+/−5% of the recited parameter.

Naturally occurring residues may be divided into classes based on common side chain properties: 1) hydrophobic: Met, Ala, Val, Leu, Ile: 2) neutral hydrophilic: Cys, Ser, Thr; 3) acidic: Asp, Glu: 4) basic: Asn, Gln, His, Lys, Arg: 5) residues that influence chain orientation: Gly, Pro: and 6) aromatic: Trp. Tyr, Phe. For example, non-conservative substitutions may involve the exchange of a member of one of these classes for a member from another class.

In making amino acid changes, the hydropathic index of amino acids may be considered. Each amino acid has been assigned a hydropathic index on the basis of its hydrophobicity and charge characteristics. The hydropathic indices are: isoleucine (+4.5); valine (+4.2); leucine (+3.8); phenylalanine (+2.8); cysteine/cystine (+2.5); methionine (+1.9): alanine (+1.8): glycine (−0.4): threonine (−0.7); serine (−0.8); tryptophan (−0.9); tyrosine (−1.3); proline (−1.6); histidine (−3.2); glutamate (−3.5); glutamine (−3.5): aspartate (−3.5); asparagine (−3.5); lysine (−3.9): and arginine (−4.5). The importance of the hydropathic amino acid index in conferring interactive biological function on a protein is generally understood in the art (Kyte et al., 1982, J. Mol. Biol. 157: 105-31). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those within ±1 are particularly preferred, and those within ±0.5 are even more particularly preferred.

It is also understood in the art that the substitution of like amino acids can be made effectively on the basis of hydrophilicity, particularly where the biologically functionally equivalent protein or peptide thereby created is intended for use in immunological disclosure, as in the present case. The greatest local average hydrophilicity of a protein, as governed by the hydrophilicity of its adjacent amino acids, correlates with its immunogenicity and antigenicity, i.e., with a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, the substitution of amino acids whose hydrophilicity values are within ±2 is preferred, those within ±1 are particularly preferred, and those within ±0.5 are even more particularly preferred. One may also identify epitopes from primary amino acid sequences on the basis of hydrophilicity.

Desired amino acid substitutions (whether conservative or non-conservative) can be determined by those skilled in the art at the time such substitutions are desired. For example, amino acid substitutions can be used to identify important residues of the HA protein, or to increase or decrease the immunogenicity, solubility or stability of the HA proteins described herein. Exemplary amino acid substitutions are shown below in Table 4.

TABLE 4
Amino Acid Substitutions

| Original Amino Acid | Exemplar/Substitutions |
|---|---|
| Ala | Val, Leu, Ile |
| Arg | Lys, Gln, Asn |
| Asn | Gln |
| Asp | Glu |
| Cys | Ser, Ala |
| Gln | Asn |
| Glu | Asp |
| Gly | Pro, Ala |
| His | Asn, Gln, Lys, Arg |
| Ile | Len, Val, Met, Ala |
| Leu | Ile, Val, Met, Ala |
| Lys | Ara, Gln, Asn |
| Met | Leu, Phe, Ile |
| Phe | Leu, Val, Ile, Ala, Tyr |
| Pro | Ala |
| Ser | Thr, Ala, Cys |
| Thr | Ser |
| Trp | Tyr, Phe |
| Tyr | Trp, Phe, Thr, Ser |
| Val | Ile, Met, Leu, Phe, Ala |

As used herein, the phrase “significantly affect a protein's activity” refers to a decrease in the activity of a protein by at least 10%, at least 20%, at least 30%, at least 40% or at least 50%. With regard to the present disclosure, such an activity may be measured, for example, as the ability of a protein to elicit neutralizing antibodies against a virus. Such activity may be measured by measuring the titer of such antibodies against virus, or by measuring the number of types, subtypes or strains neutralized by the elicited antibodies. Methods of determining antibody titers and methods of performing virus neutralization assays are known to those skilled in the art. In addition to the activities described above, other activities that may be measured include the ability to agglutinate red blood cells and the binding affinity of the protein for a cell. Methods of measuring such activities are known to those skilled in the art.

As used herein, a fusion protein is a recombinant protein containing amino acid sequence from at least two unrelated proteins that have been joined together, via a peptide bond, to make a single protein. The unrelated amino acid sequences can be joined directly to each other or they can be joined using a linker sequence. As used herein, proteins are unrelated, if their amino acid sequences are not normally found joined together via a peptide bond in their natural environment(s) (e.g., inside a cell). For example, the amino acid sequences of monomeric subunits that make up a polypeptide, and the amino acid sequences of antigen proteins are not normally found joined together via a peptide bond.

The terms individual, subject, and patient are well-recognized in the art, and are herein used interchangeably to refer to any human or other animal susceptible to infection. Examples include, but are not limited to, humans and other primates, including non-human primates such as chimpanzees and other apes and monkey species; farm animals such as cattle, sheep, pigs, seals, goats and horses: domestic mammals such as dogs and cats; laboratory animals including rodents such as mice, rats and guinea pigs; birds, including domestic, wild and game birds such as chickens, turkeys and other gallinaceous birds, ducks, geese, and the like. The terms individual, subject, and patient by themselves, do not denote a particular age, sex, race, and the like. Thus, individuals of any age, whether male or female, are intended to be covered by the present disclosure and include, but are not limited to the elderly, adults, children, babies, infants, and toddlers. Likewise, the methods of the present disclosure can be applied to any race, including, for example, Caucasian (white). African-American (black), Native American, Native Hawaiian, Hispanic, Latino, Asian, and European.

As used herein, a vaccinated subject is a subject that has been administered a vaccine that is intended to provide a protective effect against a bacteria, virus, or parasite.

As used herein, the terms exposed, exposure, and the like, indicate the subject has come in contact with a person of animal that is known to be infected with a bacteria, virus, or parasite.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates, which may need to be independently confirmed.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

It is appreciated that certain features of the disclosure, which are, for clarity. described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed. In addition, all sub-combinations are also specifically embraced by the present disclosure and are disclosed herein just as if each and every such sub-combination was individually and explicitly disclosed herein.

The disclosure is further described in the following Examples, which do not limit the scope of the disclosure described in the claims.

### 9. EXAMPLES

### 9.1. Example 1: Respiratory Syncytial Virus (RSV)

### 9.1.1. Sequences

In embodiments of the present disclosure, RSV F protein is present as a fusion protein with the first polypeptide and a linker is used, the F protein-linker sequence may comprise the following:

>DS-Cav1-foldon
(SEQ ID NO: 90)
(MELLILKANAITTILTAVTFCFASG)QNITEEFYQSTCSAVSKGYLSALR
TGWYTSVITIELSNIKENKCNGTDAKVKLIKQELDKYKNAVTELQLLMQST
PATNNRARRELPRFMNYTLNNAKKTNVTLSKKRKRRFLGFLLGVGSAIASG
VAVCKVHLHLEGEVNKIKSALLSTNKAVVSLSNGVSVLTFKVLDLKNYIDK
QLLPILNKQSCSISNIETVIEFQQKNNRLLEITREFSVNAGVTTPVSTYML
TNSELISLINDMPITNDQKKIMSNNVQIVRQQSYSIMCIIKEEVLAYVVQL
PINGVIDTPCWKLHTSPLCTTNTKEGSNICLTRTDRGWYCDNAGSVSFFPQ
AETCKVQSNRVFCDTMNSLTLPSEVNLCNVDIFNPKYDCKIMTSKTDVSSS
VITSLGAIVSCYGKTKCTASNKNRGIIKTFSNGCDYVSNKGVDTVSVGNTL
YYVNKQEGKSLYVKGEPIINFYDPLVFPSDEFDASISQVNEKINQSLAFIR
(KSDELL)GYIPEAPRDGQAYVRKDGEWVLLSTFL

In various further embodiments, the first polypeptides comprise or consist of first polypeptides having a sequence selected from the following (optional residues in parentheses):

>DS-Cav1-foldon-T33-31A
(SEQ ID NO: 91)
(MELLILKANVIATILTAVTFCFASS)QNITEEFYQSTCSAVSKGYLSALRTGWYTSVITI
ELSNIKENKCNGTDAKVKLIKQELDKYKNAVTELQLLMQSTPATNNRARRELPRFM
NYTLNNAKKTNVTLSKKRKRRFLGFLLGVGSAIASGVAVCKVLHLEGEVNKIKSALL
STNKAVVSLSNGVSVLTFKVLDLKNYIDKQLLPILNKQSCSISNIETVIEFQQKNNRLL
EITREFSVNAGVTTPVSTYMLTNSELLSLINDMPITNDQKKLMSNNVQIVRQQSYSIM
CIIKEEVLAYVVQLPLYGVIDTPCWKLHTSPLCTTNTKEGSNICLTRTDRGWYCDNA
GSVSFFPQAETCKVQSNRVFCDTMNSLTLPSEVNLCNVDIFNPKYDCKIMTSKTDVSS
SVITSLGAIVSCYGKTKCTASNKNRGIIKTFSNGCDYVSNKGVDTVSVGNTLYYVNK
QEGKSLYVKGEPIINFYDPLVFPSDEFDASISQVNEKINQSLAFIR(KSDELL)GYIPEAP
RDGQAYVRKDGEWVLLSTFLGGSMEEVVLITVPSALVAVKIAHALVEERLAACVNI
VPGLTSIYREEGSVVSDHELLLLVKTTTDAFPKLKERVKELHPYEVPEIVALPIAEGNR
EYLDWLRENTG
>DS-Cav1-T33-31A
(SEQ ID NO: 92)
(MELLILKANVIATILTAVTFCFASS)QNITEEFYQSTCSAVSKGYLSALRTGWYTSVITI
ELSNIKENKCNGTDAKVKLIKQELDKYKNAVTELQLLMQSTPATNNRARRELPRFM
NYTLNNAKKTNVTLSKKRKRRFLGFLLGVGSAIASGVAVCKVLHLEGEVNKIKSALL
STNKAVVSLSNGVSVLTFKVLDLKNYIDKQLLPILNKQSCSISNIETVIEFQQKNNRLL
EITREFSVNAGVTTPVSTYMLTNSELLSLINDMPITNDQKKLMSNNVQIVRQQSYSIM
CIIKEEVLAYVVQLPLYGVIDTPCWKLHTSPLCTTNTKEGSNICLTRTDRGWYCDNA
GSVSFFPQAETCKVQSNRVFCDTMNSLTLPSEVNLCNVDIFNPKYDCKIMTSKTDVSS
SVITSLGAIVSCYGKTKCTASNKNRGIIKTFSNGCDYVSNKGVDTVSVGNTLYYVNK
QEGKSLYVKGEPIINFYDPLVFPSDEFDASISQVNEKINQSLAFIR(KSDELL)GGSMEE
VVLITVPSALVAVKIAIIALVEERLAACVNIVPGLTSFYREEGSVVSDFIELLELNKTII
DAFPKLKERVKELHPYEVPEIVALPIAEGNREYLDWLRENTG
>DS-Cav1-foldon-T33-15B
(SEQ ID NO: 93)
(MELLILKANVIATILTAVTFCFASS)QNITEEFYQSTCSAVSKGYLSALRTGWYTSVITI
ELSNIKENKCNGTDAKVKLIKQELDKYKNAVTELQLLMQSTPATNNRARRELPRFM
NYTENNAKKTNVTLSKKRKRRFLGFLLGVGSAIASGVAVCKVLHLEGEVNKIKSALL
STNKAVVSLSNGVSVLTFKVLDLKNYIDKQLLPILNKQSCSISNIETVIEFQQKNNRLL
EITREFSVNAGVTTPVSTYMLTNSELLSLINDMPITNDQKKLMSNNVQIVRQQSYSIM
CIIKEEVLAYVVQLPLYGVIDTPCWKLHTSPLCTTNTKEGSNICLTRTDRGWYCDNA
GSVSFFPQAETCKVQSNRVFCDTMNSLTLPSEVNLCNVDIFNPKYDCKIMTSKTDVSS
SVITSLGAIVSCYGKTKCTASNKNRGIIKTFSNGCDYVSNKGVDTVSVGNTLYYVNK
QEGKSLYVKGEPIINFYDPLVFPSDEFDASISQVNEKINQSLAFIR(KSDELL)GYIPEAP
RDGQAYVRKDGEWYLLSTFLGGSMVRGIRGAITVNSDTPTSIIIATILLLEKMLEANGI
QSYEELAAVIFTVTEDLTSAFPAEAARQIGMHRVPLLSAREVPVPGSLPRVIRVLALW
NTDTPQDRVRHVYLSEAVRLRPDLESAQ
>DS-Cav1-T33-15B
(SEQ ID NO: 94)
(MELLILKANVIATILTAVTFCFASS)QNITEEFYQSTCSAVSKGYLSALRTGWYTSVITI
ELSNIKENKCNGTDAKVKLIKQELDKYKNAVTELQLLMQSTPATNNRARRELPRFM
NYTLNNAKKTNVTLSKKRKRRFLGFLLGVGSAIASGVAVCKVLHLEGEVNKIKSALL
STNKAVVSLSNGVSVLTFKVLDLKNYIDKQLLPILNKQSCSISNIETVIEFQQKNNRLL
EITREFSVNAGVTTPVSTYMLTNSELLSLINDMPITNDQKKLMSNNVQPIRQQSYSIM
CIIKEEVLAYVVQLPLYGVIDTPCWKLHTSPLCTTNTKEGSNICLTRTDRGWYCDNA
GSVSFFPQAETCKVQSNRVFCDTMNSLTLPSEVNLCNVDIFNPKYDCKIMTSKTDVSS
SVITSLGAIVSCYGKTKCTASNKNRGIIKTFSNGCDYVSNKGVDTVSVGNTLYYVNK
QEGKSLYVKGEPIINFYDPLVFPSDEFDASISQVNEKINQSLAFIR(KSDELL)GGSMVR
GIRGAITVNSDTPTSIIIATILLLEKMLEANGIQSYEELAAVIFTVTEDLTSAFPAEAARQ
IGMHRVPLLSAREVPVPGSLPRVIRVLALWNTDTPQDRVRHVYLSEAVRLRPDLESA
Q
>DS-Cav1-foldon-I53-50A
(SEQ ID NO: 95)
(MELLILKANAITTILTAVTFCFASG)QNITEEFYQSTCSAVSKGYLSALRIGWYTSVIT
IELSNIKENKCNGTDAKVKLIKQELDKYKNAVTELQLLMQSTPATNNRARRELPRFM
NYTLNNAKKTNVTLSKKRKRRFLGFLLGVGSAIASGVAVCKVLHLEGEVNKIKSALL
STNKAVVSLSNGVSVLTFKVLDLKNYIDKQLLPILNKQSCSISNIETVIEFQQKNNRLL
EITREFSVNAGVTTPVSTYMLTNSELLSLINDMPITNDQKKLMSNNVQIVRQQSYSIM
CIIKEEVLAYVVQLPLYGVIDTPCWKLHTSPLCTTNTKEGSNICLTRTDRGWYCDNA
GSVSFFPQAETCKVQSNRVFCDTMNSLTLPSEVNLCNVDIFNPKYDCKIMTSKTDVSS
SVITSLGAIVSCYGKTKCTASNKNRGIIKTFSNGCDYVSNKGVDTVSVGNTLYYVNK
QEGKSLYVKGEPIINFYDPLVFPSDEFDASISQVNEKINQSLAFIRGYIPEAPRDGQAY
VRKDGEWVLLSTFLGSGSHHHHHHHHGGSGGSGSEKAAKAEEAARKMEELFKKHK
IVAVLRANSVEEAIEKAVAVFAGGVHLIEITFTVPDADTVIKALSVLKEKGAIIGAGTV
TSVEQCRKAVESGAEFIVSPHLDEEISQFCKEKGVFYMPGVMTPTELVKAMKLGHTI
LKLFPGEVVGPQFVKAMKGPFPNVKFVPTGGVNLDNVCEWFKAGVLAVGVGSALV
KGTPDEVREKAKAFVEKIRGCTE
>DS-Cav1-I53-50A
(SEQ ID NO: 96)
(MELLILKANVIATILTAVTFCFASS)QNITEEFFYQSTCSAVSKGYLSALRTGWYTSVITI
ELSNIKENKCNGTDAKVKLIKQELDKYKNAVTELQLLMQSTPATNNRARRELPRFM
NYTLNNAKKTNVTLSKKRKRRFLGFLLGVGSAIASGVAVCKVLHLEGEVNKIKSALL
STNKAVVSLSNGIVSVLTFKVLDLKNYIDKQLLPILNKQSCSISNIETVIEFQQKNNRLL
EITREFSVNAGVTTPVSTYMLTNSELLSLINDMPITNDQKKLMSNNVQIVRQQSYSIM
CIIKEEVLAYVVQLPLYGVIDTPCWKLHTSPLCTTNTKEGSNICLTRTDRGWYCDNA
GSVSFFPQAETCKVQSNRVFCDTMNSLTLPSEVNLCNVDIFNPKYDCKIMTSKTDVSS
SVITSLGAIVSCYGKTKCTASNKNRGIIKTFSNGCDYVSNKGVDTVSVGNTLYYYNK
QEGKSLYVKGEPIINFYDPLVFPSDEFDASISQVNEKINQSLAFIRGGSGGSGSEKAAK
AEEAARKMEELFKKHKIVAVLRANSVEEAIEKAVAVFAGGVHLIEITFTVPDADTVIK
ALSVLKEKGAIIGAGTVTSVEQCRKAVESGAEFIVSPHLDEEISQFCKEKGVFYMPGV
MTPTELVKAMKLGHTILKLFPGEVVGPQFVKAMKGPFPNVKFVPTGGVNLDNVCE
WFKAGVLAVGVGSALVKGTPDEVREKAKAFVEKIRGCTE
>DS-Cav1-I32-28A
(SEQ ID NO: 97)
(MELLILKANAITTILTAVTFCFASG)QNITEEFYQSTCSAVSKGYLSALRTGWYTSVIT
IELSNIKENKCNGTDAKVKLIKQELDKYKNAVTELQLLMQSTPATNNRARRELPRFM
NYTLNNAKKTNVTLSKKRKRRFLGFLLGVGSAIASGVAVCKVLHLEGEVNKIKSALL
STNKAVVSLSNGVSVLTFKVLDLKNYIDKQLLPILNKQSCSISNIETVIEFQQKNNRLL
EITREFSVNAGVTTPVSTYMLTNSELLSLINDMPITNDQKKLMSNNVQIVRQQSYSIM
CIIKEEVLAYVVQLPLYGVIDTPCWKLHTSPLCTTNTKEGSNICLTRTDRGWYCDNA
GSVSFFPQAETCKVQSNRVFCDTMNSLTLPSEVNLCNVDIFNPKYDCKIMTSKTDVSS
SVITSLGAIVSCYGKTKCTASNKNRGIIKTFSNGCDYVSNKGVDTVSVIGNTLYYVNK
QEGKSLYVKGEKIINFYDPVFPSDEFDASISQVNEKINQSLAFIR(KSDELL)GGSGGS
GSDDARIAAIGDVDELNSQIGVLLAEPLPDDVRAALSAIQHDLFDLGGELCIPHAAIT
EDHLLRLALWLVHYNGQLPPLEEFILPGGARGAALAHVCRTVCRRAERSIKALGASE
PLNIAPAAYVNLLSDLLFVLARVLNRAAGGADVLWDRTRAH
>DS-Cav1-Tr-foldon-T33-31A
(SEQ ID NO: 101)
QNITEEFYQSTCSAVSKGYLSALRTGWYTSVITIELSNIKENKCNGTDAKVKLIKQEL
DKYKNAVTELQLLMQSTPATNNRARRELPRFMNYTLNNAKKTNVTLSKKRKRRFL
GFLLGVGSAIASGVAVCKVLHLEGEVNKIKSALLSTNKAVVSLSNGVSVLTFKVLDL
KNYIDKQLLPILNKQSCSISNIETVIEFQQKNNRLLEITREFSVNAGVTTPVSTYMLTNS
ELLSLINDMPITNDQKKLMSNNVQIVRQQSYSIMCIIKEEVLAXVVQLPLYGVIDTPC
WKLHTSPLCTTNTKEGSNICLTRTDRGWYCDNAGSVSFFPQAETCKVQSNRVFCDT
MNSLTLPSEVNLCNVDIFNPKYDCKIMTSKTDVSSSVITSLGAIVSCYGKTKCTASNK
NRGIIKTFSNGCDYVSNKGVDTVSVGNTLYYVNKQEGKSLYVKGEPIINFYDPLVFPS
DEFDASISQVNEKINQSLAFIRGYIPEAPRDGQAYVRKDGEWLLSTFLGGSMEEVVL
ITVPSALVAVKIAHALVEERLAACVNIVPGLTSIYREEGSVVSDHELLLLVKTTTDAFP
KLKERVKELHPYEVPEIVALPIAEGNREYLDWLRENTG
>DS-Cav1-Tr-T33-31A
(SEQ ID NO: 102)
QNITEEFYQSTCSAVSKGYISALRTGWYTSVITIELSNIKENKCNGTDAKVKLIKQEL
DKYKNAVTELQLLMQSTPATNNRARRELPRFMNYTLNNAKKTNVTLSKKRKRRFL
GFLLGVGSAIASGVAVCKVLHLEGEVNKIKSALLSINKAVVSLSNGVSVLTFKVLDL
KNYIDKQLLPILNKQSCSISNIETVIEFQQKNNRLLEITREFSVNAGVTTPVSTYMLTNS
ELLSLINDMPITNDQKKLMSNNVQIVRQQSYSIMCIIKEEVLAYVVQLPLYGVIDTPC
WKLHTSPLCTTNTKEGSNICLTRTDRGWYCDNAGSVSFFPQAETCKVQSNRVFCDT
MNSLTLPSEVNLCNVDIFNPKYDCKIMTSKTDVSSSVITSLGAIVSCYGKTKCTASNK
NRGIIKTFSNGCDYVSNKGVDTVSVGNTLYYVNKQEGKSLYVKGEPIINFYDPLVFPS
DEFDASISQVNEKINQSLAFIRGGSMEENVVLITVPSALVAVKIAHALVEERLAACVNIV
PGLTSIYREEGSVVSDHELLLLVKTTTDAFPKLKERVKMELHPYEAVPEIVALPIAEGNRE
YLDWLRENTG
>DS-Cav1-Tr-foldon-T33-15B
(SEQ ID NO: 103)
QNITEEFYQSTCSAVSKGYLSALRTGWYTSVITIELSNIKENKCNGTDAKVKLIKQEL
DKYKNAVTELQLLMQSTPATNNRARRELPRFMNYTLNNAKKTNVTLSKKRKRRFL
GFLLGVGSAIASGVAVCKVLHLEGEVNKIKSALLSTNKAVVSLSNGVSVLTFKVLDL
KNYIDKQLLPILNKQSCSISNIETVIEFQQKNNRLLEITREFSVNAGVTTPVSTYMLTNS
ELLSLINDMPITNDQKKLMSNNVQIVRQQSYSIMCIIKEEVLAYVVQLPLYGVIDTPC
WKLETSPLCTTNTKEGSNICLTRTDRGWYCDNAGSVSFFPQAETCKVQSNRVFCDT
MNSLFLPSEVNLCNVDIFNPKYDCKIMTSKTDVSSSVITSLGAIVSCYGKTKCTASNK
NRGIIKTFSNGCDYVSNKGVDTVSVGNTLYYVNKQEGKSLYVKGEPIINFYDPLVFPS
DEFDASISQVNEKINQSLAFIRGYIPEAPRDGQAYVRKDGEWVLLSTFLGGSMVRGIR
GAITVNSDTPTSIIIATILLLEKMLEANGIQSYEELAAVIFTVTEDLTSAFTAEAARQIG
MHRVPLLSAREVPVPGSLPRVIRVLALWNTDTPQDRVRHVYLSEAVRLRPDLESAQ
>DS-Cav1-Tr-T33-15B
(SEQ ID NO: 104)
QNITEEFYQSTCSAVSKGYLSALRTGWYTSVITIELSNIKENKCNGTDAKVKLIKQEL
DKYKNAVTELQLLMQSTPATNNRARRELPRFMNYTLNNAKKTNVTLSKKRKRRFL
GFLLGVGSAIASGVAVCKLHLEGEVNKIKSALLSTNKAVVSLSNGVSVLTFKVLDL
KNYIDKQLLPILNKQSCSISNIETVIEFQQKNNRLLEITREFSVNAGVTTPVSTYMLTNS
ELLSLINDMPITNDQKKLMSNNVQIVRQQSYSIMCIIKEEVLAYVVQLPLYGVIDTPC
WKLHTSPLCTTNTKEGSNICLTRTDRGWYCDNAGSVSFFPQAETCKVQSNRVECDT
MNSULTLPSEVNLCNVDIFNPKYDCKIMTSKTDVSSSVITSLGAIVSCYGKTKCTASNK
NRGIIKTFSNGCDYVSNKGVDTVSVGNTLYYVNKQEGKSLYVKGEPIINFYDPLVFPS
DEFDASISQVNEKINQSLAFIRGGSMVRGIRGAITVNSDTPTSIIIATILLLEKMLEANGI
QSYEELAAVIFTVTEDLTSAFPAEAARQIGMHRVPLLSAREVPVPGSLPRVIRVLALW
NTDTPQDRVRHVYLSEAVRLRPDLESAQ
>DS-Cav1-Tr-foldon-I53-50A
(SEQ ID NO: 105)
QNITEEFYQSTCSAVSKGYLSALRTGWYTSVITIELSNIKENKCNGTDAKVKLIKQEL
DKYKNAVTELQLLMQSTPATNNRARRELPRFMNYTLNNAKKTNVTLSKKRKRRFL
GFLLGVGSAIASGVAVCKVLHLEGEVNKIKSALLSTNKAVVSLSNGVSVLTFKVLDL
KNYIDKQLLPILNKQSCSISNIETVIEFQQKNNRLLEITREFSVNAGVTTPVSTYMLTNS
ELLSLINDMPITNDQKKLMSNNVQIVTRQQSYSIMCIIKEEVLAYVVQLPLYGVIDTPC
WKLHTSPLCTTNTKEGSNICLTRTDRGWYCDNAGSVSFFPQAETCKVQSNRVFCDT
MNSLTLPSEVNLCNVDIFNPKYDCKIMTSKTDVSSSVITSLGAIVSCYGKTKCTASNK
NRGIIKTFSNGCDYVSNKGVDTVSVGNTLYYVNKQEGKSLYVKGEPIINFYDPLNFPS
DEFDASISQVNEKINQSLAFIRGYIPEAPRDGQAYVRKDGEWVLLSTFLGSGSHHHHH
HHHGGSGGSGSEKAAKAEEAARKMEELFKKHKIVAVIRANSVEEAIEKAVAVFAGG
VHLIEITFTVPDADTVIKALSVLKEKGAIIGAGTVTSVEQCRKAVESGAEFIVSPHLDE
EISQFCKEKGVFYMPGVMTPTELVKAMKLGHTILKLFPGEVVGPQFVKAMKGPFPN
VKFVPTGGVNLDNVCEWFKAGVLAVGVGSALVKGTPDEVREKAKAFVEKIRGCTE
>DS-Cav1-Tr-I53-50A
(SEQ ID NO: 106)
QNITEEFYQSTCSAVSKGYLSALRTGWYTSVITIELSNIKENKCNGTDAKVKLIKQEL
DKYKNAVTELQLLMQSTPATNNRARRELPRFMNYTLNNAKKTNVTLSKKRKRRFL
GFLLGVGSAIASGVAVCKVLHLEGEVNKIKSALLSTNKAVVSLSNGVSVLTFKVLDL
KNYIDKQLLPILNKQSCSISNIETVIEFQQKNNRLLEITREFSVNAGVTTPVSTYMLTNS
ELLSLINDMPITNDQKKLMSNNVQIVRQQSYSIMCIIKEEVLAYVVQLPLYGVIDTPC
WKLHTSPLCTTNTKEGSNICLTRTDRGWYCDNAGSVSFFPQAETCKVQSNRVFCDT
MNSLTLPSEVNLCNVDIFNPKYDCKIMTSKTDVSSSNIVITSLGAIVSCYGKTKCTASNK
NRGIIKTFSNGCDYVSNKGVDTVSVGNTLYYVNKQEGKSLYVKGEPIINFYDPLVFPS
DEFDASISQVNEKINQSLAFIRGGSGGSGSEKAAKAEEAARKMEELFKKHKIVAVLR
ANSVEEAIEKAVAVFAGGVHLIEITFTVPDADTVIKALSVLKEKGAIIGAGTVTSVEQ
CRKAVESGAEFIVSPHLDEEISQFCKEKGVFYMGVMTPTELVKAMKLGHTILKLFP
GEVVGPQFVKAMKGPFPNVKFVPTGGVNLDNVCEWFKAGVLAVGVGSALVKGTP
DEVREKAKAFVEKIRGCTE
>DS-Cav1-Tr-I32-28A
(SEQ ID NO: 107)
QNITEEFYQSTCSAVSKGYLSALRTGWYTSVITIELSNIKENKCNGTDAKVKLIKQEL
DKYKNAVTELQLLMQSTPATNNRARRELPRFMNYTLNNAKKTNVTLSKKRKRRFL
GFLLGVGSAIASGVAVCKVLHLEGEVNKIKSALLSTNKAVVSLSNGVSVLTFKVLDL
KNYIDKQLLPILNKQSCSISNIETVIEFQQKNNRLLEITREFSVNAGVTTPVSTYMLTNS
ELLSLINDMPITNDQKKLMSNNVQIVRQQSYSIMCIIKEEVLAYVVQLPLYGVIDTPC
WKLHTSPLCTTNTKEGSNICLTRTDRGWYCDNAGSVSFFPQAETCKVQSNRVFCDT
MNSLTLPSEVNLCNVDIFNPKYDCKIMTSKTDVSSSVITSLGAIVSCYGKTKCTASNK
NRGIIKTFSNGCDYVSNKGVDTVSVGNTLYYVNKQEGKSLYVKGEPIINFYDPINFPS
DEFDASISQVNEKINQSLAFIRGGSGGSGSDDARIAAIGDVDELNSQIGVLLAEPLPDD
VRAALSAIQHDLFDLGGELCIPGHAAITEDHLLRLALWLVHYNGQLPPLEEFILPGGA
RGAALAHVCRTVCRRAERSIKALGASEPLNIAPAAYVNLLSDLLFVLARVLNRAAGG
ADVLWDRTRAH

### 9.1.2. Methods

### Expression and Screening Of Trimeric Building Blocks Comprising an F Protein and a Trimeric Assembly Domain.

Human codon-optimized sequences for trimeric building blocks including and lacking DS-Cav I fusions were ordered from Genscript. Building blocks for single-component nanostructures (i.e., 13-01) were cloned into the pcDNA3.1 vector (ThermoFisher Scientific) containing one CMV promoter, while building blocks for two-component nanostructures (e.g., I53-50) were cloned into the pBudCE4.1 vector (ThermoFisher Scientific) containing both CMV and EF-la promoters. Recombinant proteins were expressed by transient transfection of Expi293F cells (ThermoFisher Scientific) using polyethylenimine (PEI). Cell cultures were harvested five days post-transfection by centrifugation. Secreted proteins were analysed by ELISA, using either direct coating of the cell supernatants or by sandwich ELISA. Briefly, 96-well MaxiSorp plates (Nunc) were coated with cell supernatant for direct ELISA or murine anti-His tag monoclonal antibody (ThermoFisher Scientific) for sandwich ELISA. Secreted proteins were detected using the human Palivizumab, MPE8, RSDS, and D25 monoclonal antibodies. Transfected Expi293F cells were fixed and permeabilized with BD cytofix/eytoperm (BD Biosciences), incubated with human Palivizumab, MPE8, and D25 monoclonal antibodies, and stained with Alexa Fluor 647-conjugated anti-human IgG antibody (Jackson ImmunoResearch). Stained cells were counted with a FACS Fortessa flow cytometer (BD Biosciences). Analysis was performed with FlowJo software. Cell lines were routinely tested for mycoplasma contamination.

### Expression and Purification of DS-Cav1-I53-50A

Lentivirus was produced by transient transfection of 293T (ATCC) cells using linear 25-kDa polyethyleneimine (PEI: Polysciences). Briefly, 4×10⁶ cells were plated onto 10 cm tissue culture plates. After 24 h. 3 μg of psPAX2, 1.5 μg of pMD2G (Addgene plasmid #12260 and #12259, respectively) and 6 μg of lentiviral vector plasmid were mixed in 500 pi diluent (5 mM HEPES, 150 mM NaCl, pH=7.05) and 42 μl of PEI (1 mg/ml) and incubated for 15 min. The DNA/PEI complex was then added to the plate drop-wise. Lentivirus was harvested 48 h post-transfection and concentrated 100-fold by low-speed centrifugation at 8000 g for 18 h. Transduction of the target cell line was carried out in 125 mL shake flasks containing 10×10⁶ cells in 10 mL of growth media. 100 uL of 100×lentivirus was added to the flask and the cells were incubated with shaking (225 rpm) at 37° C., in 8% CO₂ for 4-6 h. 20 mL of growth media was added to the shake flask after 4-6 h.

Transduced cells were expanded every other day to a density of 1×10⁶ cells/ml until a final culture size of 4 L was reached. The media was harvested after 17 days of total incubation after measuring final cell concentration (˜5×10⁶ cells/mL) and viability (˜90% viable). Culture supernatant was harvested by low-speed centrifugation to remove cells from the supernatant. NaCl and NaN₃ were added to final concentrations of 250 mM and 0.02%, respectively. The supernatant was loaded over one 5 mL HisTrap FF Crude column (GE Healthsciences) at 5 ml/min by an AKTA Pure (GE Healthsciences). The nickel elution was applied to a HiLoad 16/600 Superdex 200 pg column (GE Healthsciences) to further purify the target protein by size-exclusion chromatography. The size-exclusion purified target protein was snap frozen in liquid nitrogen and stored at −80° C.

In Vitro Assembly of DS-Cav1-bearing Nanostructures

100% valency particles (20 DS-Cav1 trimers per icosahedral nanostructure) were prepared by mixing DS-Cav1-foldon-I53-50A trimers and I53-50B.4PT1 pentamers at 50 μM each and incubating with rocking overnight at 4° C. In some cases, assembled nanostructures were purified from excess components remaining in the in vitro assembly reaction using a GE Sephacryl S-500 HR 16/60 column in a buffer comprising 25 mM Tris pH 8, 250 mM NaCl, 5% glycerol. Sample load and SEC fractions were analyzed by SDS-PAGE in the presence and absence of reducing agent. Peak fractions were pooled, concentrated using a GE Vivaspin 20 30kDa MWCO centrifugal filter, and quantified using an Agilent 8454 spectrophotometer.

66% valency particles (˜14 DS-Cav1 trimers per icosahedral nanostructure) were prepared by mixing DS-Cav1-foldon-I53-50A trimers, I53-50A trimers, and I53-50B.4PosT1 pentamers at 50, 25, and 75 μM, respectively. 33% valency particles (−7 DS-Cav1 trimers per icosahedral nanostructure) were prepared by mixing DS-Cav1-foldon-I53-50A trimers, I53-50A trimers, and I53-50B.4PosT1 pentamers at 25, 50, and 75 μM, respectively. The in vitro assembly reactions were allowed to incubate with rocking overnight at 4° C. In some cases, assembled nanostructures were purified from excess components remaining in the in vitro assembly reaction using a GE Sephacryl S-500 HR 16/60 column in a buffer comprising 25 mM Tris pH 8, 250 mM NaCl, 5% glycerol. Sample load and SEC fractions were analyzed by SDS-PAGE in the presence and absence of reducing agent. Peak fractions were pooled, concentrated using a GE Vivaspin 20 30kDa MWCO centrifugal filter, and quantified using an Agilent 8454 spectrophotometer after centrifuging at ˜21,000 g for 10 minutes at 4° C. Samples were then transferred to cryogenic tubes in 1 mL aliquots at 1.1 mg/mL for the 33% valency particles and 0.6 mg/mL for the 66% valency particles, flash frozen in liquid nitrogen, and stored at −80° C.

Electron Microscopy of DS-Cav1-bearing Nanostructures

Samples were prepared for negative stain EM by diluting to 0.01 mg/mL using 25 mM Tris pH 8, 250 mM NaCl, 5% glycerol and 3.5 μL was incubated on a glow-discharged, copper, carbon-coated grid for 20 seconds before blotting away the liquid with a piece of Whatman No. I filter paper. Within seconds of blotting away the sample, a 3.5 μL droplet of stain (2% w/v uranyl formate) was deposited and blotted away immediately, and then a second cycle of staining/blotting was performed.

### Circular Dichroism (CD) Spectropolarimetry

CD spectra from F proteins (0.5 mg ml **) were recorded on a Chirascan spectropolarimeter (Applied Photophysics) over the wavelength range of 195 to 260 nm at a bandwidth of 1 nm, step size of 0.5 nm, and 1 s per step. The spectra in the far-ultraviolet region required an average of three scans and were subtracted from blank spectra performed with buffer. Thermal denaturation was monitored by performing scans at intervals of 1° C., after equilibration for 1 min at each temperature. Data were fitted to a simple first order curve. The values of ΔA222 are represented on the y axis as the percentage of the values recorded at 20° C.

### Enzyme-linked Immunosorbent Assay (ELISA)

To test specific binding of antibody or sera, 96-well MaxiSorp plates (Nunc) were coated with serial dilutions of tissue culture supernatants from cells expressing trimeric building blocks comprising F proteins and a trimeric assembly domain or 2 μg ml⁻¹ of the following purified proteins: Ds-Cav I with foldon, Ds-Cav1 fused to a trimeric first polypeptide or DS-Cav 1-displaying nanostructures. Plates were blocked with 1% bovine serum albumin (BSA) and incubated with titrated antibodies (D25. MPE8, Palivizumab. RSD5) or murine sera followed by AP-conjugated goat anti-human IgG (Southern Biotech. 2040-04) or goat anti-mouse IgG (Southern Biotech, 1030-04). Plates were then washed with PBS buffer (Gibco. Invitrogen), 0.05% Tween-20 and substrate (p-NPP, Sigma) was added and plates were read at 405 nm.

### Surface Plasmon Resonance (SPR)

The experiments were carried out at 25° C, on a ProteON XPR-36 instrument (Bio-Rad Laboratories) in a PBS buffer (Gibco, Invitrogen), 0.05% Tween-20. The D25 mAb was immobilized on a GLM sensor chip surface through amine coupling at 1000 response units (RU) and a blank surface with no protein was created under identical coupling conditions for use as a reference. Monoclonal antibodies (D25, MPE8, Palivizumab and 131-2a) were injected at a flow rate of 100 μl/min, at concentrations of 50 nM in different sensor channels. The data were processed using Proteon software and double referenced by subtraction of the blank surface and buffer only injection before local fitting of the data.

### Vaccination and Serological Analysis

Female BALB/c mice 6-9 wk of age were obtained from Harlan Laboratories Inc. All procedures were performed in accordance with guidelines of the Swiss Federal Veterinary Office and after obtaining local ethical approval. Mice were immunized i.p, with 100 μL of immunogen formulation on day 0, 14, and 28. Priming infection at day 0 was performed with the Murine TLR9 ligand agonist (ODN 1668, InvivoGen). Mice were bled on day 10, 20 and 40, and antigen-and site-specific IgG titers were measured in the serum by ELISA. Neutralizing titers were also determined on HEp-2 cell as described below.

### Virus Neutralization Assay and Microscopy Analysis

Confluent layers of HEp-2 cells in 96-well flat-bottom plates were infected with a fixed amount of Human Respiratory Syncytial Virus with Green Fluorescent Protein (RSV strain A2. Vira Trec#R121) at MOI of 1. 48 hours post-infection the cells were stained with Hoechst (Sigma#H6024) and images were acquired on BD Pathway bioimaging system. Percentage of the infected cells was automatically calculated by BD Atto Vision software. The number of infected cells was plotted as dose response curves by plotting the relative infected cells against the antibodies dilutions.

Stability of DS-Cav1-bearing Nanostructures

Physical stability of the prefusion conformation of designed DS-Cav1-foldon-I53-50 was assessed by incubating protein at various concentrations in a PCR cycler with heated lid at 80° C., for 1 h. Residual prefusion conformation was evaluated by direct coating of the protein and ELISA with the prefusion-specific antibody D25.

### Statistical Analysis

No statistical methods were used to predetermine sample size. Data were analyzed with Prism 6 (GraphPad Software) using the two-tailed non-parametric Mann-Whitney U test for two groups comparison, or Kruskall-Wallis test (and Dunn's posttest) when three or more groups were compared.

### 9.1.3. Results

Trimeric building blocks comprising an F protein and a trimeric assembly domain

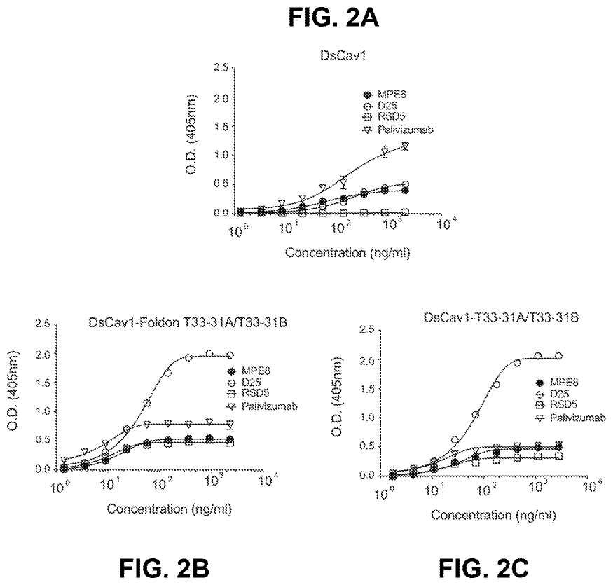

Several trimeric building blocks, each comprising an F protein genetically fused to a trimeric assembly domain, were found to be secreted from HEK293F cells with their F proteins in a well-folded, prefusion conformation as judged by prefusion-specific monoclonal antibody binding in ELISA assays. FIG. 2 shows an example of ELISA data analyzing the supernatant of HEK293F cells expressing DS-Cav1-foldon, DS-Cav1-foldon-T33-31A, and DS-Cav1-T33-31A. Several other trimeric building blocks yielded detectable secretion of well-folded, prefusion F proteins.

Expression and Purification of DS-Cav1-foldon-I53-50A

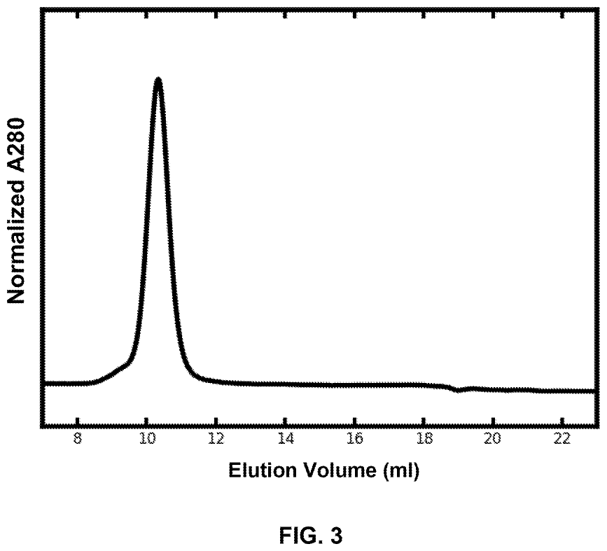

A lentiviral vector encoding DS-Cav1-foldon-I53-50A was used to transduce HEK293F cells for large-scale expression. The secreted protein was purified from tissue culture supernatants by immobilized metal affinity chromatography and size exclusion chromatography. Size exclusion chromatograms (FIG. 3) indicated that the purified protein formed a single, monodisperse species.

### Expression and Purification of I53-50B.4PT1

I53-50B.4PT1, a pentameric protein comprising a second assembly domain that interacts with the trimeric assembly domain in I53-50A or DS-Cav1-foldon-I53-50A to drive assembly of icosahedral I53-50-based nanostructures, was expressed and purified as described in Bale et al, and patent publication US20160122392 A1.

In Vitro Assembly and Characterization of DS-Cav1-bearing I53-50 Nanostructures

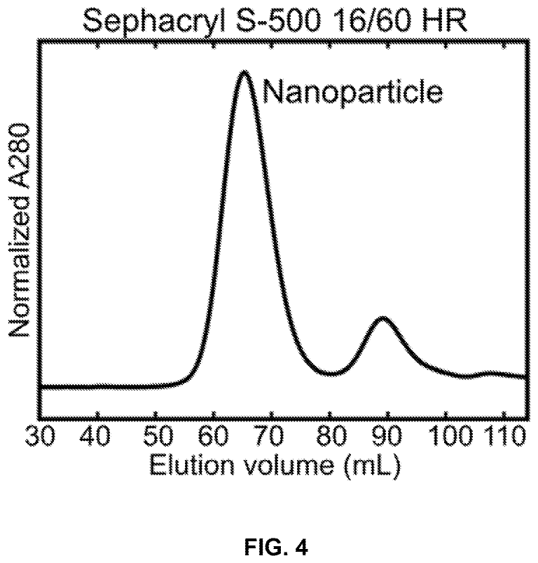

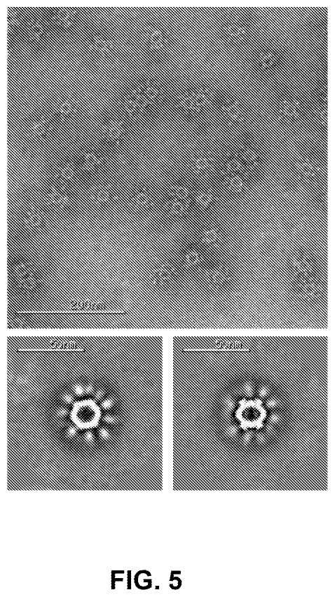

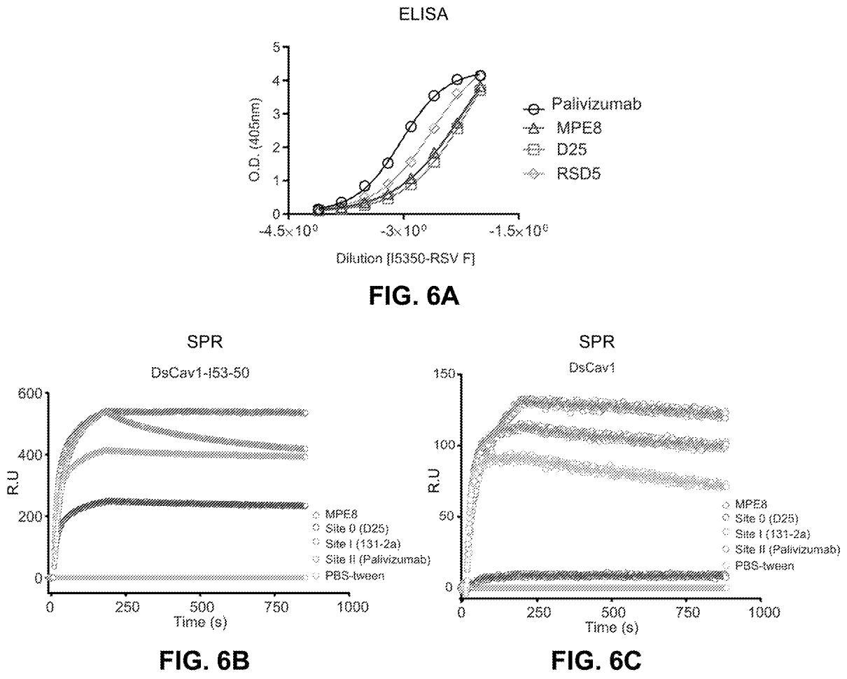

I53-50 is a 120-subunit two-component nanostructure with icosahedral symmetry comprising 20 trimeric (I53-50A) and 12 pentameric (I53-50B) building blocks, as recently described by Bale et al. The N terminus of I53-50A is exposed on the exterior of the I53-50 nanostructure, which enables the display of antigens on the nanostructure exterior through genetic fusion to the I53-530A N terminus. Purified DS-Cav1-foldon-I53-50A and I53-50B.4PT1 were assembled in vitro to form 120-subunit icosahedral nanostructures displaying various amounts of DS-Cav1 on the nanostructure exteriors by mixing the two purified proteins in various molar ratios. In separate preparations, nanostructures displaying DS-Cav1 at valencies of 100% (20 trimers). 66% (˜14 timers), and 33% (˜7 trimers) were prepared as described above. The species present in the in vitro assembly reactions after overnight incubation were assessed by several techniques, including size exclusion chromatography-multi-angle light scattering (SEC-MALS), dynamic light scattering, and UV/vis spectroscopy. Assembled, 120-subunit nanostructures were purified from the in vitro assembly reactions using size exclusion chromatography (an example chromatogram obtained using the 100% valency nanostructures is presented in FIG. 4). The purified nanostructures were characterized by negative stain electron microscopy, which revealed fields of monodiperse particles in which DS-Cav1 was clearly visible as spikes projecting outward from the core icosahedral I53-50 assembly (an example micrograph obtained using the 100% valency particles is presented in FIG. 5). ELISA assays using monomclonal antibodies specific to the prefusion conformation confirmed that the DS-Cav1 thus displayed on the nanostructure exteriors was well-folded and antigenically intact (FIG. 6). Surface plasmon resonance experiments evaluating the kinetics of monoclonal antibody binding revealed that antibody dissociation from the 100% valency DS-Cav1-foldon-I53-50 nanostructures was slower than from DS-Cav1-foldon trimers, likely due to avidity effects deriving from the multivalent presentation of DS-Cav1 on the nanostructure exterior (FIG. 6). Together, these experiments confirmed that the DS-Cav1-foldon-I53-50 nanostructures formed monodisperse, icosahedral nanostructures that display well-folded, antigenically intact DS-Cav1 trimers on their exteriors. These findings motivated experiments to evaluate the utility of the DS-Cav1-foldon-I53-50 nanostructures as immunogens for inducing humoral immune responses against DS-Cav1 in animals.

Immunogenicity of DS-Cav1-foldon-I53-50 Nanostructures

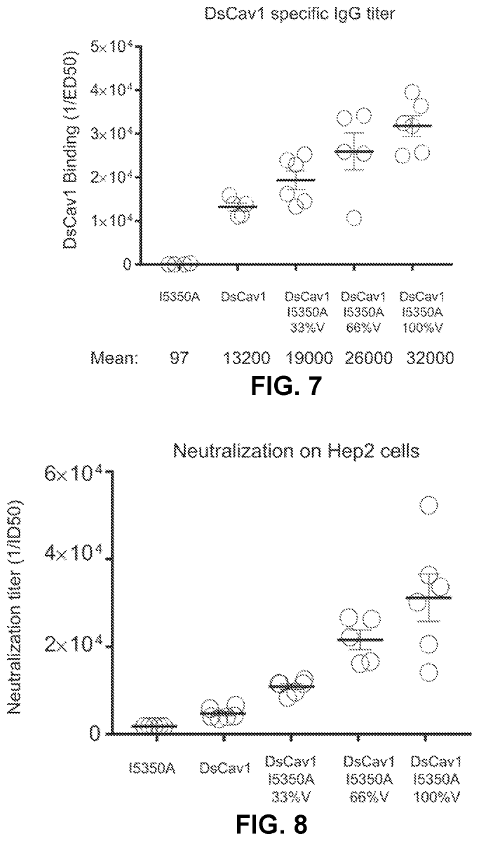

The DS-Cav1-foldon-I53-50 nanostructures displaying DS-Cav1 at 33%, 66%, and 100% valency were injected into mice using a prime-boost strategy as described above. Additional groups of mice were injected with trimeric DS-Cav1-foldon as a benchmark for the humoral immune response induced against DS-Cav1 by the nanostructures or I53-50 nanostructures lacking displayed DS-Cav1 as negative controls for a DS-Cav1 specific response. ELISA assays of serum extracted from the mice at defined timepoints after the injections were used to measure DS-Cav1 specific antibody titers present in the sera of the injected animals (FIG. 7). As expected, sera from animals injected with the I53-50 nanostructures lacking displayed DS-Cav1 did not contain antibodies specific to DS-Cav1. Trimeric DS-Cav1-foldon induced DS-Cav1-specific antibodies, in accordance with previous results (McClellan et al.). The 33%, 66%, and 100% valency DS-Cav1 nanostructures all induced higher DS-Cav1-specific antibody titers than trimeric DS-Cav1-foldon, with the antibody titers increasing with increasing DS-Cav1 valency. DS-Cav1-specific titers were roughy 2.5-fold higher on average in mice injected with 100% valency DS-Cav1-foldon-I53-50 nanostructures compared to DS-Cav1. These results demonstrate that immunogens in which paramyxovirus F proteins are multivalently displayed on self-assembling protein nanostructures can induce higher humoral immune responses when injected into animals.

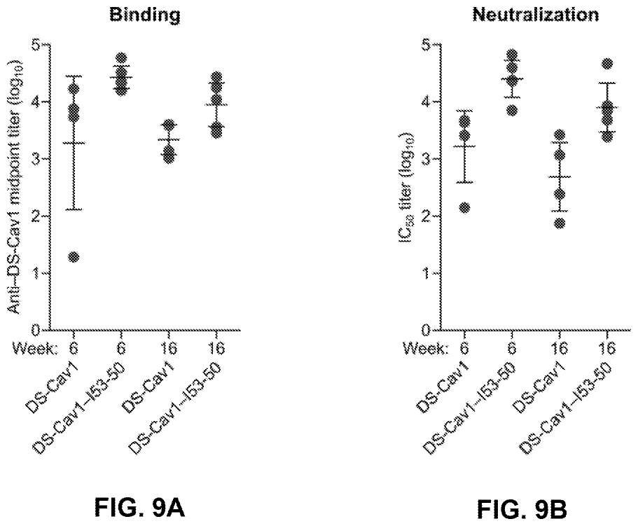

The sera from the mice injected with the series of immunogens described above was also evaluated for the presence of neutralizing antibody titers using the standard neutralization assay in HEp-2 cells (FIG. 8). The trend in serum neutralizing antibody titers correlated highly with the trend observed in DS-Cav1-specific binding antibody titers. Sera from animals injected with the I53-50 nanostructures lacking displayed DS-Cav1 did not neutralize virus, consistent with the lack of DS-Cav1-specific antibodies in these sera. The sera from animals injected with trimeric DS-Cav-1-foldon neutralized virus with an average titer (1/ID₅₀) of 3,030. The 33%, 66%, and 100% valency DS-Cav1-I53-50 nanostructures induced higher neutralizing antibody titers than trimeric DS-Cav1-foldon, with average titers of 9.400. 20,000, and 30,500, respectively. These results demonstrate that the higher response induced by immunogens in which paramyxovirus F proteins are multivalently displayed on self-assembling protein nanostructures result in more effective virus neutralization.

### Physical Stabilization of DS-Cav1 by Fusion to I53-50A

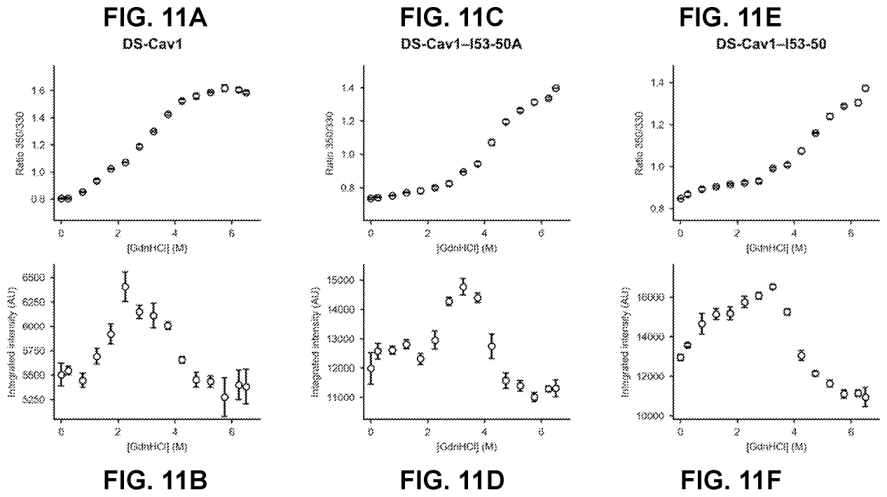

Given the key antigenic properties of prefusion F, we used two orthogonal approaches to measure the physical stability of DS-Cav1 when fused to I53-50A and/or when further assembled into the icosahedral nanostructure. The first assay measured the retention of binding by a prefusion-specific mAb (D25) after thermal stress, an approach that has been used previously to characterize prefusion F stability (Mclellan et al. 2013; Joyce et al. 2016: Krarup et al. 2015). Samples of trimeric DS-Cav1, trimeric DS-Cav1-I53-50A, and DS-Cav1-I53-50 nanostructures containing equivalent concentrations (50 nM) of DS-Cav1 were split into four aliquots and incubated at 20, 50, 70 or 80° C. for 1 hour. After cooling to room temperature, D25 binding was assayed by surface plasmon resonance (SPR). We found that all samples bound D25 equivalently at 20 and 50° C., but lost most of their reactivity to D25 after 1 hour at 80° C., as previously reported for DS-Cav1 (Mclellan et al. 2013; Joyce et al. 2016) (FIG. 10). Interestingly, while D25 was also unable to bind trimeric DS-Cav1 incubated at 70° C. for 1 hour, trimeric DS-Cav1-I53-50A and the DS-Cav1-I53-50 nanostructures retained 50 and 80% of their respective binding signals (FIG. 10). While the multivalent nature of the DS-Cav1-I53-50 nanostructures complicates direct quantitative comparisons to trimeric DS-Cav1, these results indicate that genetic fusion to the I53-50A trimer further stabilizes the prefusion conformation of DS-Cav1, and suggest that this increased stability is maintained in the context of the assembled nanostructure immunogen.

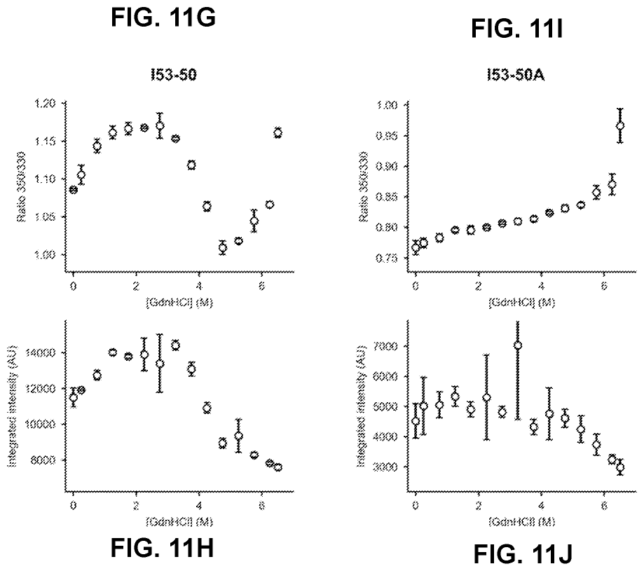

We used chemical denaturation in guanidine hydrochloride (GdnHCl), monitored by intrinsic tryptophan fluorescence, as a second, antibody-independent technique to evaluate physical stability. Analyzing fluorescence emission from DS-Cav1 incubated in 0-6.5 M GdnHCl revealed that the protein undergoes two subtly distinct transitions, one between 0.25 and 2.25 M GdnHCl and another between 2.25 and 5.75 M (FIGS. 11A-11J). In contrast, only a single transition is apparent for trimeric DS-Cav1-I53-50A, occurring between 2.25 and 6.25 M GdnHCl (FIGS. 11A-11J). It is unclear at present whether the transition at lower [GdnHCl] observed for DS-Cav1 is absent from trimeric DS-Cav1-I53-50A or simply shifted to higher [GdnHCl]. However, it is clear that the native conformation of DS-Cav1 is stabilized by genetic fusion to trimeric I53-50A, mirroring the results obtained by measuring D25 binding after thermal stress. Comparing the data for the DS-Cav1-I53-50 nanostructure and the I53-50 nanostructure alone (lacking fused DS-Cav1) indicated that the stabilization is maintained upon assembly to the icosahedral nanostructure (FIGS. 11A-11J). The source of this effect is likely the extreme stability of the I53-50A trimer. I53-50A is derived from the KDPG aldolase of the hyperthermophilic bacterium T. maritima and only began to exhibit changes in fluorescence at very high (≥5.75 M) GdnHCl concentrations (FIGS. 11A-11J).

We made addition constructs to assess the number of GS repeats and the need for a stabilization domain such as the Foldon moiety.

### Sequence Information

| IPD Name | MS (Da) | Construct information |
|---|---|---|
| RSV_F-10 | 74005.38 | DS-Cav1-8GS-HclExt-50A (SEQ ID NO: 108) |
| RSV_F-11 | 74293.64 | DS-Cav1-12GS-HelExt-50A (SEQ ID NO: 109) |
| RSV_F-12 | 74551.87 | DS-Cav1-16GS-HelExt-50A (SEQ ID NO: 110) |
| RSV_F-13 | 77212.97 | DS-Cav1-foldon-10GS-HelExt-50A (SEQ ID NO: 111) |
| RSV_F-14 | 77558.28 | DS-Cav1-foldon-15GS-HelExt-50A (SEQ NO: 112) |
| RSV_F-15 | 77933.62 | DS-Cav1-foldon-20GS-HelExt-50A (SEQ ID NO: 113) |

>RSV_F-10
(SEQ ID NO: 108)
QNITEEFYQSTCSAVSKGYLSALRIGWYTSVITIELSNIKENKCNGTDAKVKLIKQEL
DKYKNAVTELQLLMQSTPATNNRARRFLGFLLGVGSAIASGVAVCKVLHLEGEVNK
IKSALLSTNKAVVSLSNGVSVLTFKVLDLKNYIDKQLLPILNKQSCSISNIETVIEFQQK
NNRLLEITREFSVNAGIVTTPVSTYMLTNSELLSLINDMPITNDQKKLMSNNVQIVRQQ
SYSIMCIIKEEVLAYVVQLPLYGVIDTPCWKLHTSPLCTTNTKEGSNICLTRTDRGWY
CDNAGSVSFFTQAETCKVQSNRVFCDTMNSLTLPSEVNLCNVDIFNPKYDCKIMTSK
TDVSSSVITSLGAIVSCYGKTKCTASNKNRGIIKTFSNGCDYVSNKGVDTVSVGNTLY
YVNKQEGKSLYVKGEPIINFYDPLVFPSDEFDASISQVNEKINQSLAFIRGSGGSGSGE
KAAKAEEAARKMEELFKKHKIVAVLRANSVEEAIEKAVAVFAGGVHLIEITFTVPDA
DTVIKALSVLKEKGAIIGAGTVTSVEQCRKAVESGAEFIVSPHLDEEISQFCKEKGVFY
MPGVMTPTELVKAMKLGHTILKLFPGEVVGPQFVKAMKGPFPNVKFVPTGGVNLD
NVCEWFKAGVLAVGVGSALVKGTPDEVREKAKAFVEKIRGCTE
>RSV_F-11
(SEQ ID NO: 109)
QNITEEFQSTCSAVSKGYLSALRTGWYTSVITIELSNIKENKCNGTDAKVKLIKQEL
DKYKNAVTELQLLMQSTPATNNRARRFLGFLLGVGSAIASGVAVCKVLHLEGEVNK
IKSALLSTNKAVVSLSNGVSVLTFKVLDLKNYIDKQLLPILNKQSCSISNIETVIEFQQK
NNRLLEITREFSVNAGVTTPVSTYMLTNSELLSLINDMPITNDQKKLMSNNVQIVRQQ
SYSIMCIIKEEVLAYVVQLPLYGVIDTPCWKLHTSPLCTTNTKEGSNICLTRTDRGWY
CDNAGSVSFFPQAETCKVQSNRVFCDTMNSLTLPSEVNLCNVDIFNPKYDCKIMTSK
TDVSSSVITSLGAIVSCYGKTKCTASNKNRGIIKTFSNGCDYVSNKGVDTVSVGNTLY
YVNKQEGKSLYVKGEPIINFYDPLVFPSDEFDASISQVNEKINQSLAFIRGSGGSGSGS
GGSEKAAKAEEAARKMEELFKKHKIVAVIJRANSVEEAIEKAVAVFAGGVHLIEITFT
VPDADTVIKALSVLKEKGAIIGAGTVTSVEQCRKAVESGAEFIVSPHLDEEISQFCKEK
GVFYMPGVMTPTELVKAMKLGHTILKLFPGEVVGPQFVKAMKGPFPNVKFVPTGG
VNLDNVCEWFKAGVLAVGVGSALVKGTPDEVREKAKAFVEKIRGCTE
>RSV_F-12
(SEQ ID NO: 110)
QNITEEFYQSTCSAVSKGYLSALRTGWYTSVITIELSNIKENKCNGTDAKVKLIKQEL
DKYKNAVTELQLLMQSTPATNNRARRFLGFLLGVGSAIASGVAVCKVLHLEGEVNK
IKSALLSTNKAVVSLSNGVSVLTFKVLDLKNYIDKQLLPILNKQSCSISNIETVIEFQQK
NNRLLEITREFSVNAGVTTPVSTYMLTNSELLSLINDMPITNDQKKLMSNNVQIVRQQ
SYSIMCIIKEEVLAYVVQLPLYGVIDTPCWKLHTSPLCTTNTKEGSNICLTRTDRGWY
CDNAGSVSFFPQAETCKVQSNRVFCDTMNSLTLPSEVNLCNVDIFNPKYDCKIMTSK
TDVSSSVITSLGAIVSCYGKTKCTASNKNRGIIKITSNGCDYVSNKGVDTVSVGNTLY
YVNKQEGKSLYVKGEPIINFYDPLVFPSDEFDASISQVNEKINQSLAFIRGSGGSGSGS
GGSGSGGEKAAKAEEAARKMEELFKKHKIVAVLRANSVEEAIEKAVAVFAGGVHLI
EITFTVPDADTVIKALSVLKEKGAIIGAGTVTSVEQCRKAVESGAEFIVSPHLDEEISQF
CKEKGVFYMPGVMTPTELVKAMKLGHTILKLFPGEVVGPQFVKAMKGPFPNVKFVP
TGGVNLDNVCEWFKAGVLAVGVGSALVKGTPDEVREKAKAFVEKIRGCTE
>RSV_F-13
(SEQ ID NO: 111)
QNITEEFYQSTCSAVSKGYLSALRTGWYTSVITIELSNIKENKCNGTDAKVKLIKQEL
DKYKNAVTELQLLMQSTPATNNRARRFLGFLLGVGSAIASGVAVCKVLHLEGEVNK
IKSALLSTNKAVVSLSNGVSVLTFKVLDLKNYIDKQLLPILNKQSCSISNIETVIEFQQK
NNRLLEITREFSVNAGVTTPVSTYMLTNSELLSLINDMPITNDQKKLMSNNVQIVRQQ
SYSIMCIIKEEVLAYVVQLPLYGVIDTPCWKLHTSPLCTTNTKEGSNICLTRTDRGWY
CDNAGSVSFFPQAETCKVQSNRVFCDTMNSLTLPSEVNLCNVDIFNPKYDCKIMTSK
TDVSSSVITSLGAIVSCYGKTKCTASNKNRGIIKTFSNGCDYVSNKGVDTVSVGNTLY
YVNKQEGKSLYVKGEPIINFYDPLVFPSDEFDASISQVNEKINQSLAFIRGSGGSGSGS
GEKAAKAEEAARKMEELFKKHKIVAVLRANSVEEAIEKAVAVFAGGVHLIEITFTNP
DADTVIKALSVLKEKGAIIGAGTVTSVEQCRKAVESGAEFIVSPHLDEEISQFCKEKG
VFYMPGVMTPTELVKAMKLGHTILKLFPGEVVGPQFVKAMKGPFPNVKFVPTGGV
NLDNVCEWFKAGVLAVGVGSALVKGTPDEVREKAKAFVEKIRGCTE
>RSV_F-14
(SEQ ID NO: 112)
QNITEEFYQSTCSAVSKGYLSALRTGWYTSVITIELSNIKENKCNGTDAKVKLIKQEL
DKYKNAVTELQLLMQSTPATNNRARRFLGFLLGVGSAIASGVAVCKVLHLEGEVNK
IKSALLSTNKAVVSLSNGVSVLTFKVLDLKNYIDKQLLPILNKQSCSISNIETVIEFQQK
NNRLLEITREFSVNAGVTTPVSTYMLTNSELLSLINDMPITNDQKKLMSNNVQIVRQQ
SYSIMCIIKEEVLAYVVQLPLYGVIDTPCWKLHTSPLCTTNTKEGSNICLTRTDRGWY
CDNAGSVSFFPQAETCKVQSNRVFCDTMNSLTLPSEVNLCNVDIFNPKYDCKIMTSK
TDVSSSVITSLGAIVSCYGKTKCTASNKNRGIIKTFSNGCDYVSNKGVDTVSVGNTLY
YVNKQEGKSLYVKGEPIINFYDPLVFPSDEFDASISQVNEKINQSLAFIRGYIPEAPRD
GQAYVRKDGEWVLLSTFLGSGGSGSGSGGSGSGEKAAKAEEAARKMEELFKKHKIV
AVLRANSVEEAIEKAVAVFAGGVHLIEITFTVPDADTVIKALSVLKEKGAIIGAGTVT
SVEQCRKAVESGAEFIVSPHLDEEISQFCKEKGVFYMPGVMTPTELVKAMKLGHTIL
KLFPGEVVGPQFVKAMKGPFPNVKFVPTGGVNLDNVCEWFKAGVLAVGVGSALVK
GTPDEVREKAKAFVEKIRGCTE
>RSV_F-15
(SEQ ID NO: 113)
QNITEEFYQSTCSAVSKGYLSALRTGWYTSVITIELSNIKENKCNGTDAKVKLIKQEL
DKYKNAVTELQLLMQSTPATNNRARRFLGFLLGVGSAIASGVAVCKVLHLEGEVNK
IKSALLSTNKAVVSLSNGVSVLTFKVLDLKNYIDKQLLPILNKQSCSISNIETVIEFQQK
NNRLLEITREFSVNAGVTTPVSTYMLTNSELLSLINDMPITNDQKKLMSNNVQIVRQQ
SYSIMCIIKEEVLAYVVQLPLYGVIDTPCWKLHTSPLCTTNTKEGSNICLTRTDRGWY
CDNAGSVSFFPQAETCKVQSNRVFCDTMNSLTLPSEVNLCNVDIFNPKYDCKIMTSK
TDVSSSVITSLGAIVSCYGKTKCTASNKNRGIIKTFSNGCDYVSNKGVDTVSVGNTLY
YVNKQEGKSLYVKGEPIINFYDPLVFPSDEFDASISQVNEKINQSLAFIRGYIPEAPRD
GQAYVRKDGEWVLLSTFLGSGGSGSGSGGSGSGGSSGSEKAAKAEEAARKMEELFK
KHKIVAVLRANSVEEAIEKAVAVFAGGVHLIEITFTVPDADTVIKALSVLKEKGAIIG
AGTVTSVEQCRKAVESGAEFIVSPHLDEEISQFCKEKGVFYMPGVMTPTELVKAMKL
GHTILKLFPGEVVGPQFVKAMKGPFPNVKVPTGGVNLDNVCEWFKAGVLAVGVG
SALVKGTPDEVREKAKAFVEKIRGCTE

Studies were based on expression yield in a small-scale transient transfection. Plasmids capable of expressing the relevant constructs were transformed into NEB 5α E. coli cells and selected on LB+carbenicillin agar plates. 1 mL cultures were prepared by inoculating TB media with a bacterial colony and again selecting with 50 ug/mL carbenicillin. A Qiagen Mini Prep kit was used to purify plasmid from the E. coli cultures in accordance with their protocol. Expi293F™ Cells (ThermoFisher) were cultured in Expi293™ Expression Medium (ThermoFisher) supplemented with penicillin (100 u/mL) and streptomycin (100 μg/mL) at 8% CO₂, 37° C., and 125 rpm shaking.

On the day prior to transfection, cells were seeded at a concentration of 2E6 cells/mL. On the day of transfection, cells were counted by a Countess II (ThermoFisher) with trypan blue to determine cell viability. Cell concentration was adjusted to 2.5E6 cells/mL, and cells where plated into untreated 12-well plates (Corning) in 1 mL volumes. 1 μg of DNA plasmid were transfected per each well using Expifectamine™ (ThermoFisher), following the manufacturer's directions. Enhancers, components of ThermoFisher's Expifectamine™ Transfection Kit, were added 18 hours after transfection. The 1 mL cultures were harvested 5 days post-transfection, and the cells were pelleted from the supernatant by centrifugation at 1,500×g for 5 minutes at 4° C. Supernatants were filtered through a 0.45 μM filter with a PVDF membrane.

Filtered supernatants containing DS-Cav1-I53-50A constructs were denatured and boiled for 10 minutes at 95° C, for 10 minutes in 2× Laemmli buffer with 2-mercaptoethanol. SDS-PAGE separated the sample fractions, which were then transferred to a nitrocellulose membrane and probed with palivizumab, followed with a secondary antibody, anti-human conjugated to HRP. Blot was imaged using Clarity Western ECL Blotting Substrate (Bio-Rad).

Filtered supernatants containing DS-Cav1-I53-50A constructs were bound to Nunc MaxiSorp 96-well plates in a two-fold dilution series. The pre-fusion conformation-specific antibody D25 was used to detect DS-Cav1-I53-50A, followed by a secondary anti-human antibody conjugated to HRP. Protein yield was determined colorimetrically via the substrate TMB and absorbances were collected at 450 nm.

The expression yields and binding of the prefusion-specific mAb D25 (data not shown) indicate that all constructs express well and are in the prefusion conformation. As is known to those of skill in the art, a heterologous trimerization domain (e.g., the foldon) is typically required for proper expression and folding of prefusion F constructs. Our results indicate that the I53-50A nanostructure component can support the expression and proper folding of DS-Cav1 without the use of a trimerization domain like the foldon. Binding of D25 to these constructs suggests that they are antigenically intact and would be expected to induce potent immune responses, including neutralizing antibodies, similarly to nanostructures comprising the DS-Cav1-foldon-I53-50 fusion polypeptide.

### 9.2. Example 2: Cytomegalovirus (CMV)

Protein-based vaccines for CMV are described, for example, in U.S. Patent Pub. Nos. US 2016/0159864 A1 and US 2017/0369532 A1; International Patent Pub No. WO 2016/092460 A3; and Kirchmeier al. Enveloped virus-like particle expression of human cytomegalovirus glycoprotein B antigen induces antibodies with potent and broad neutralizing activity. Clin Vaccine Immunol. 2014; 21(2): 174-80. The homotrimer complex of gB, the trimeric gH/gL/gO complex, or the pentameric gH/gL/pUL128/pUL130/pUL131A complex are considered the three major targets for CMV vaccination.

The first of these targets, gB, forms a trimeric structure which comprises several hydrophobic surfaces. The C terminus of the extracellular domain of gB is proximal to the transmembrane region and lies near the 3-fold axis of the molecule. See Chandramouli et al. Structure of HCMV glycoprotein B in the postfusion conformation bound to a neutralizing human antibody. Nat Commun. 2015 Sep. 14;6:8176. By substitution of the transmembrane region of gB for a linker, the gB protein of CMV is N-terminally linked to a nanostructure having a free N terminus at or near the 3-fold axis of the nanostructure. The resulting nanostructure has displays 20 copies of the gB trimer on its surface and effectively elicits a immune response to CMV gB. Mutations to the gB protein as described in International Patent Pub No. WO 2016/092460 A3, improve the solubility and immunogenicity of the nanostructure-based vaccine.

The second of these targets, the trimeric gH/gL/g(complex, and the third of these targets, the pentameric gH/gL/pUL128/pUL130/pUL131A, form by mutually exclusive interactions of the envelope glycoproteins gH/gL with either gO or pUL128/pUL130/pUL131A. See Ciferri et al. Structural and biochemical studies of HCMV gH/gL/g( )and Pentamer reveal mutually exclusive cell entry complexes. Proc. Natl. Acad. Sci. U.S.A. 112, 1767-1772 (2015). The gH component is targeted by antibodies neutralizing infection of both fibroblasts and endothelial/epithelial cells. The UL region contains the binding sites for potently neutralizing antibodies of epithelial and endothelial cells infection.

The gH component is expressed as a gene fusion to a nanostructure polypeptide and either gL/gO or gL/pUL128/pUL130/pUL131A are co-expressed. The expressed proteins self-assemble into either gH/gL/gO or gH/gL/pUL128/pUL130/pUL131A nanostructure-based vaccines, respectively. Expression and correct folding of the nanostructure is assessed by binding of the MSL-109 antibody of an Fab fragment thereof to the nanostructure. Correct folding and antigenicity of the pentameric complex is assessed using antibodies and Fab fragments described in Chandramouli et al. Structural basis for potent antibody-mediated neutralization of human cytomegalovirus Sci. Immunol. 2, eaan 1457 (2017).

### 9.3. Example 3: Epstein-Barr Virus (EBV)

Epstein-Barr virus (EBV) represents a major global health problem. Though it is associated with infectious mononucleosis and ˜200,000 cancers annually worldwide, a vaccine is not available. The major target of immunity is EBV glycoprotein 350/220 (gp350) that mediates attachment to B cells through complement receptor 2 (CR2/CD21). See Kanekiyo et al. Rational Design of an Epstein-Barr Virus Vaccine Targeting the Receptor-Binding Site. Cell 162(5): 1090-1100 (2015). The gp350 ectodomain or the D₁₂₃ fragment of gp350 is expressed as a gene fusion to a nanostructure polypeptides as either an N-terminal or C-terminal fusion. The resulting gene fusions are expressed, assembled, and formulated into nanostructure-based vaccines. Antigenicity is determined using the monoclonal antibodies 72A1 and 2L10.

1. A nanostructure, comprising a first plurality of polypeptides, wherein:
the first plurality of polypeptides are arranged according to at least one symmetry operator;
the nanostructure comprises a first plurality of antigens;
each of the first plurality of the antigens has a proximal end and a distal end; and
the proximal ends of the antigens are each attached to a member of the first plurality of polypeptides.

2. The nanostructure of claim 1, further comprising a second plurality of polypeptides, wherein the second plurality of polypeptides is attached to the first plurality of polypeptides.

3. The nanostructure of claim 1, further comprising a second plurality of antigens.

4. The nanostructure of claim 1, further comprising a second plurality of antigens, wherein:
each of the second plurality of second antigens has a proximal end and a distal end, and
the proximal ends of the second antigens are each attached to a member of the second plurality of polypeptides

5. The nanostructure of claim 1, wherein the proximal ends of the antigens are the N termini of the antigens.

6. The nanostructure of claim 1, wherein the proximal ends of the antigens are the C termini of the antigens.

7. The nanostructure of claim 1, wherein the plurality of antigens is a plurality of antigenic proteins or antigenic fragments thereof.

8. The nanostructure of claim 7, wherein the antigenic protein is selected from a polypeptide of SEQ ID NOs: 52-56, 59-85,88 and 90-113 or a variant thereof.

9. The nanostructure of claim 7, wherein the antigenic protein is at least 75, 80, 85, 90, 95, or 99% identical to a polypeptide selected from SEQ ID NOs: 52-56, 59-85, 88 and 90-113.

10. The nanostructure of claim 7, wherein the antigenic protein is selected from the group consisting of:
a) HIV Env,
b) RSVF,
c) EBV gp350,
d) CMV gB,
e) CMV UL128,
f) CMV UL130,
g) CMV UL131A,
h) CMV gH,
i) CMV gL,
j) Lyme OspA,
k) Pertussis toxin,
l) Dengue E,
m) SARS S,
n) MERS S,
o) Zaire ebolavirus GP,
p) Sudan ebolavirus GP,
q) Marburg virus GP,
r) Hanta virus Gn,
s) Hanta virus Gc,
t) HepB surface antigen,
u) Measles H,
v) Zika envelope domain III,
w) Malaria CSP,
x) Malaria Pfs25,
y) Nipah virus F,
z) Nipah virus G,
aa) Rotavirus VP4,
bb) Rotavirus VP8*,
cc) hMPV F,
dd) hMPV G,
ee) PVF,
ff) PV HN,
gg) MenB fHbp,
hh) MenB NadA, and
ii) MenB NHBA.

11-19. (canceled)

20. A vaccine comprising a nanostructure of claim 1, wherein the vaccine is capable of eliciting a neutralizing antibody response to an infectious agent.

21-26. (canceled)

27. A method of generating immunity to an infectious agent in a subject, comprising administering a vaccine of claims 20.

28-34. (canceled)

35. A pharmaceutical composition comprising a vaccine of claim 20.

36. A method of making the nanostructure of claim 1 by in vitro assembly, comprising:
expressing the first plurality of polypeptides in a first recombinant expression system,
expressing the first plurality of antigens in a second recombinant expression system,
purifying the first plurality of polypeptides,
purifying the first plurality of antigens, and
mixing the first plurality of polypeptides and the first plurality of antigens;
thereby generating the nanostructure.

37-40. (canceled)

41. The method of claim 36, wherein the first plurality of polypeptides and the first plurality of antigens are encoded by a single open reading frame.

42. The method of making of claim 41, wherein the single open reading frame encodes a fusion protein of the polypeptide and the antigen.

43. The method of making of claim 42, wherein the single open reading frame encodes a self-cleaving peptide.
